(19) United States
(12) Patent Application Publication
(10) Pub. No.: US 2026/0286302 A1
(43) Pub. Date: Sep. 24, 2026

(54) METHODS FOR PRODUCING A SUPER CENTRAL MEMORY CD8 T CELL SUBTYPE BY POLY(ADP-RIBOSE) POLYMERASE (PARP) INHIBITION AND USES THEREOF

(71) Applicant: Georgetown University, Washington, DC (US)

(72) Inventors: Samir N. Khleif, Washington, DC (US); Seema Gupta, Washington, DC (US); Wael Traboulsi, Washington, DC (US)

(73) Assignee: Georgetown University, Washington, DC (US)

(21) Appl. No.: 19/473,713

(22) PCT Filed: Apr. 12, 2024
(86) PCT No.: PCT/US2024/024381

§ 371 Date: Oct. 8, 2025

Related U.S. Application Data
(60) Provisional application No. 63/459,176, filed on Apr. 13, 2023.

(51) Int. Cl. C12N 5/0783; A61K 31/166; A61K 31/407; A61K 31/416; A61K 31/4184; A61K 31/454; A61K 31/496; A61K 31/502; A61K 31/5025; A61K 31/55; A61K 31/551; A61K 35/17; A61K 35/768; A61K 38/19; A61K 39/00; A61K 40/11; A61K 40/31; A61K 45/06; A61P 35/00

(52) U.S. Cl. CPC C12N 5/0636; A61K 31/166; A61K 31/407; A61K 31/416; A61K 31/4184; A61K 31/454; A61K 31/496; A61K 31/502; A61K 31/5025; A61K 31/55; A61K 31/551; A61K 35/17; A61K 35/768; A61K 38/19; A61K 39/0011; A61K 40/11; A61K 40/31; A61K 45/06; A61P 35/00; C12N 2501/999

(57) ABSTRACT

A unique CD8 T cell central memory subtype obtained by treatment of CD8 T cells with a poly(ADP-ribose) polymerase (PARP) inhibitor (PARPi) is described. These cells, referred to as super-central memory CD8 T cells (T_{CM}^{sup}) or PARPi-induced memory T cells, are significantly more efficient for cancer therapy than traditional central memory T cells (T_{CM}) generated by tumor antigens. Methods of using the T_{CM}^{sup} cells for enhancing cancer immunotherapy, enhancing vaccine-induced immunity, and treating cancer are described.

### CROSS REFERENCE TO RELATED APPLICATIONS

This application claims the benefit of U.S. Provisional Application No. 63/459,176, filed Apr. 13, 2023, which is herein incorporated by reference in its entirety.

### FIELD

This disclosure concerns methods of producing super central memory CD8+ T cells with enhanced potency and a greater recall response. Use of the super central memory CD8+ T cells, such as for adoptive cell therapy, enhancing the efficacy of immunotherapies and vaccines, and treating cancer are also described.

### INCORPORATION OF ELECTRONIC SEQUENCE LISTING

The electronic sequence listing, submitted herewith as an XML file named 8031-110054-02.xml (5,507 bytes), created on Apr. 12, 2024, is herein incorporated by reference in its entirety.

### BACKGROUND

Enhancing T cell function and preventing terminal differentiation and exhaustion in the tumor microenvironment (TME) is one of the goals to improve cancer immunotherapy. Among the different T cell populations, it is well established that less differentiated CD8 T cells with higher metabolic fitness including stem cell-like memory (T_{SCM}) and central memory cells (T_{CM}) can provide superior anti-tumor immunity than the more-differentiated effector memory counterparts (Klebanoff, et al., Clin Cancer Res 17(16):5343-5352, 2011; Verma, et al., Nat Immunol. 22(1):53-66, 2021). Correlation between high densities of tumor-infiltrating CD8+/CD45RO⁺ memory cells and disease-free/overall survival of patients was established for various cancers (Zhou et al., J Oral Maxillofac Surg 77(3):536-545, 2019; Klebanoff et al., Proc Natl Acad Sci USA, 102(27):9571-9576, 2005). Also, active/adoptive cell transfer (ACT) of less differentiated CD8 T cells or CAR T cells has a better clinical outcome (Klebanoff et al., Immunol Rev 211: p. 214-224, 2006; Busch et al., Semin Immunol 28(1):28-34, 2016). Targeted therapies have been shown to have immune modulatory effect (Offringa et al., Nat Rev Drug Discov 21(11):821-840, 2022). Therefore, understanding their effect on T cell reprograming is important for the development of rational approaches for combination strategies to enhance immunotherapy outcome.

Because of its role as a key molecule in the DNA repair mechanism, inhibition of PARP (Poly(ADP-ribose) polymerase) has been used as a therapeutic strategy against tumors associated with deficiency or mutation in DNA damage repair (DDR) genes by inducing synthetic lethality (Lord and Ashworth, Science 355(6330):1152-1158, 2017; Murai et al., Cancer Res 72(21):5588-5599, 2012). Several PARP inhibitors (PARPi) such as olaparib, talazoparib, niraparib, veliparib, rucaparib, and saruparib, targeting PARP1 and PARP2 have been used in clinical practice with a significant clinical outcome in BRCA deficient tumors such as breast and ovarian cancers (Sonnenblick et al., Nat Rev Clin Oncol 12(1):27-41, 2015; Kim and Nam, Int J Mol Sci 23(15):8412, 2022). Interestingly, both PARP1 and PARP2 play an important role in T cell biology; while PARP2 is important for double positive T cell (CD4+CD8+ T cell) development in the thymus (Yelamos et al., EMBO J 25(18):4350-4360, 2006), PARP1 is induced upon TCR activation, modulating expression of multiple target genes, such as the genes involved in T cell activation (Saenz et al., BMC Genomics 9:171, 2008; Valdor et al., Mol Immunol 45(7):1863-1871, 2008).

Several groups have reported an indirect effect of PARPi by increasing the recruitment and function of CD8 T cells to the TME through activation of STING pathway and increase in INF-type I and chemokine secretion (Pantelidou et al., Cancer Discov 9(6):722-737, 2019; Shen et al., Cancer Res 79(2):311-319, 2019; Bustos et al., Cell Biosci 13(1):200, 2023) Also, previous studies have demonstrated that tumor re-challenge of mice showing complete response to olaparib treatment results in complete tumor rejection [25], indicating a potential indirect implication of PARPi-induced memory CD8 T cells in eliciting anti-tumor effect. Furthermore, PARPi has also been shown to affect other immune cells within the TME including, macrophages, myeloid-derived suppressor cells (MDSCs) and cancer associated fibroblasts (CAFs), promoting either an immune enhancement or immune suppressor milieu (Sun et al., Mol Ther 29(1):60-74, 2021) that may also indicate an indirect effect on CD8 T cell function. The direct effect of PARPi on CD8 T cells, however, is not understood and hence needs further investigations to understand its effects on CD8 T cell activation, function, and differentiation.

### SUMMARY

The present disclosure reports the direct effect of PARPi on CD8 T cells. It is demonstrated that inhibition of PARP generates a unique subpopulation of superior central memory cells (T_{CM}^{sup}) which leads to the induction of a strong anti-tumor activity. PARPi results in the enhanced trapping of PARP in the nucleus, suppresses cell cycle progression, and enhances metabolic fitness by modulating the SIRT-1/FOXO1 pathway, leading to the modification of gene expression related to memory and fatty acid oxidation (FAO). These PARPi-generated memory CD8 T cells are distinct from antigen-generated central memory (T_{CM}) cells in having significantly higher memory potential including antigen-specific recall responses and more potent anti-tumor functionality. Accordingly, PARPi-induction of T_{CM}^{sup} represents an important strategy to enhance anti-tumor immunotherapy both ex vivo and in vivo.

Provided herein are ex vivo methods of producing super central memory CD8+ T (T_{CM}^{sup}) cells (also referred to herein as “PARPi-induced memory T cells”). The T_{CM}^{sup} cells are characterized by expression of CD62L and CD44, such as CD62L^{int}CD44^{int} or CD62L^{hi}CD44^{hi}, or for human cells CD45RO and CD62L or CD45RO and CCR7, such as CD45RO^{int} and CD62L^{int}, or CD45RO^{int} and CCR7^{int}, CD45RO^{hi}CD62L^{hi} or CD45RO^{hi}CCR7^{hi}. In some aspects, the method includes culturing CD8+ T cells in the presence of a PARP inhibitor (PARPi), such as a small molecule PARPi. In some aspects, the PARPi is an inhibitor of PARP1, PARP2, or both. In some examples, the PARPi is olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide. The CD8+ T cells can be obtained or isolated from a variety of different types of samples, such as but not limited to, samples containing peripheral blood or tumor-infiltrating lymphocyte samples. The CD8+ T cells can also be engineered CD8+ T cells.

Further provided are methods of treating cancer in a subject by culturing CD8+ T cells ex vivo with a PARP inhibitor to produce super central memory CD8+ T cells that express CD62L and CD44 (such as CD62L^{int}CD44^{int} or CD62L^{hi}CD44^{hi}), CD45RO and CD62L (such as CD45RO^{int} and CD62L^{int} or CD45RO^{hi} and CD62L^{hi}), or CD45RO and CCR7 (such as CD45RO^{int} and CCR7^{int} or CD45RO^{hi} and CCR7^{hi}), and adoptively transferring the super central memory CD8+ T cells to the subject. In some aspects, the CD8+ T cells are autologous CD8+ T cells. In other aspects, the CD8+ T cells are allogeneic CD8+ T cells. In some aspects, the PARPi is a small molecule inhibitor, such as an inhibitor of PARP1, PARP2, or both. In some examples, the PARPi is olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide.

Also provided are methods of enhancing an immune response to a vaccine, such as a cancer vaccine or a vaccine against a microbial pathogen (e.g., a bacterial vaccine or a viral vaccine). In some aspects, the method includes administering to the subject the vaccine and a therapeutically effective amount of a PARPi, such as a small molecule PARPi. In some examples, the PARPi is olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide. In other aspects, the method includes administering to the subject the vaccine and a therapeutically effective amount of super central memory CD8+ T cells that express CD62L and CD44 (such as CD62L^{int}CD44^{int} or CD62L^{hi}CD44^{hi}). In some examples, the method further includes producing the super central memory CD8+ T cells that express CD62L and CD44 by culturing CD8+ T cells with a PARPi. The CD8+ T cells can be autologous or allogeneic CD8+ T cells.

Methods of treating cancer by administering to the subject an immunotherapy (e.g., a tumor antigen-specific antibody, a tumor-targeted chimeric antigen receptor T cell, a checkpoint inhibitor, a cancer vaccine, an oncolytic virus, one or more cytokines, or agonist antibodies for costimulatory molecules/receptors) and a therapeutically effective amount of a PARPi are also provided. In some examples, the PARPi is a small molecule inhibitor, such as olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide. In other aspects, the cancer is treated by administering to the subject an immunotherapy and a therapeutically effective amount of super central memory CD8+ T cells that express CD62L and CD44 (such as CD62L^{int}CD44^{int} or CD62L^{hi}CD44^{hi}), CD45RO and CD62L (such as CD45RO^{int} and CD62L^{int} or CD45RO^{hi} and CD62L^{hi}), or CD45RO and CCR7 (such as CD45RO^{int} and CCR7^{int} or CD45RO^{hi} and CCR7^{hi}). In some examples, the method further includes producing the super central memory CD8+ T cells that express CD62L and CD44 by culturing CD8+ T cells with a PARPi. The CD8+ T cells can be autologous or allogeneic CD8+ T cells.

The foregoing and other features of this disclosure will become more apparent from the following detailed description of several aspects which proceeds with reference to the accompanying figures.

### BRIEF DESCRIPTION OF THE DRAWINGS

FIGS. 1A-1H: PARP inhibition enhances an anti-tumor immune response by modulating CD8 T cell function and differentiation. (FIG. 1A) Tumor growth rate, mouse survival and SK plots in a TC-1 tumor model after various treatments (Untreated (UT); Vaccine (Vacc), PARPi, Vaccine plus PARPi (Vacc+PARPi)). Tumor growth and survival data are the average of three independent experiments. The error bars indicate the s.e.m. For tumor growth, statistical analysis was performed by unpaired, one-tailed Student's t-test. The P value in the tumor growth graph in FIG. 1A is between Vaccine and Vaccine plus PARPi (***P≤0.001) and for FIG. 1B, *P<0.05. Survival in various groups was compared using log-rank (Mantel-Cox) tests, **P≤0.01; ***P≤0.001; ****P≤0.0001. (FIGS. 1B-1H), Analysis of immune infiltrate in tumors 2-3 days after second vaccination. Number of total CD8⁺ T cells (FIG. 1B); E7⁺CD8⁺ T cells (FIG. 1C); INFγ⁺ E7⁺CD8⁺ T cells (FIG. 1D); Gzm B⁺ E7⁺CD8⁺ T cells (FIG. 1E); central memory E7⁺CD8⁺ T cells (FIG. 1F); FACS analysis of E7⁺CD8⁺ T cells for memory characteristics (T_{CM}; CD62L⁺CD44⁺) (FIG. 1G); MFI KI67 in CD8⁺ T cells (FIG. 1H), in the TME of variously treated mice as shown in the figure. Representative data from three experiments are shown. Each symbol corresponds to one experiment. The error bars indicate the s.e.m. Statistical analysis was performed by unpaired, one-tailed Student's t-test. *P≤0.05; **P≤0.01; ***P≤0.001; ****P<0.0001; NS, not significant.

FIGS. 2A-2K: PARPi increases memory and cytotoxic markers in CD8 T cells. MFI of Ki67 in pMel-1 CD8 cells after activation with or without PARPi for 48 h (FIG. 2A). Percent of CD62L^{hi}CD44^{hi} from total T_{CM} CD8 T cells after activation with or without PARPi for 48 h (FIG. 2B). FACS analysis of variously in vitro-activated pMel-1 CD8⁺ T cells with or without PARPi for memory characteristics (T_{CM}; CD62L⁺CD44⁺) at 48 h and 96 h of treatment (FIGS. 2C-2D). FACS analysis of variously in vitro-activated WT CD8⁺ T cells with or without PARPi for 48 h for memory characteristics (T_{CM}; CD62L⁺CD44⁺) (FIG. 2 E). MFI of Eomes, CCR7 and Bcl2 in pMel-1 CD8 T cells after activation with or without PARPi for 48 h T_{CM} CD8⁺ T cells (FIGS. 2F-2H). MFI of IFNγ⁺ in T_{CM} in pMel-1 CD8 cells after activation with or without PARPi for 48 h (FIG. 2I). MFI of Gzm B+ in T_{CM} in pMel-1 CD8 cells after activation with or without PARPi for 48 h (FIG. 2J). Percent of CD62L^{hi}CD44^{hi} from total T_{CM} CD8 T cells at 96 h of treatment (FIG. 2K). Representative data from one of the three experiments performed in triplicate are shown as mean±s.e.m. Statistical analysis was performed by unpaired, one-tailed Student's t-test (***P≤0.001; ****P≤0.0001).

FIGS. 3A-3G: PARPi reprograms the CD8 memory compartment in mouse and human. (FIG. 3A) FACS analysis of variously in vitro-activated pMel-1 CD8⁺ T with or without PARPi for 48 h cells for memory characteristics (T_{CM}; CD62L⁺CD44⁺). Percent of CD62L^{hi}CD44^{hi} from total T_{CM} CD8 T cells at 48 h of treatment (FIG. 3B). MFI of IFNγ in CD62L^{hi}CD44^{hi} after activation with or without PARPi for 48 h (FIG. 3C). MFI of Gzm B in CD62L^{hi}CD44^{hi} after activation with or without PARPi for 48 h (FIG. 3D). Representative data from one of the three experiments performed in triplicate are shown as mean±s.e.m. Statistical analysis was performed by unpaired, one-tailed Student's t-test (***P≤0.001; ****P≤0.0001). Percent of central memory (T_{CM}: CD45RO+CD62L+, CD45RO+CCR7+) CD8 T cells in patients pre and post treatment (FIG. 3E). Percent of Ki67⁺CD8 T cells in patients pre and post treatment (FIG. 3F). CD62L expression on central memory CD8 T cells in patients pre and post treatment (FIG. 3G).

FIGS. 4A-4F: PARPi leads to the development of a novel subpopulation of memory CD8 T cells with high recall capability. MFI of IFNγ and Gzm B in T_{CM} CD8⁺ T cells after re-challenge with different concentrations of gp100 peptide for 24 h (FIGS. 4A-4B). Experimental plan (FIG. 4C). MFI of IFNγ in CD8 T cells collected 3 days after vaccination with gp100 peptide (FIG. 4D). MFI of Gzm B in CD8 T cells collected 3 days after vaccination with gp 100 peptide (FIG. 4E). Tumor growth rate in the TC-1 model after active cell transfer of T_{CM} cells (CD62L⁺CD44⁺) or CD62L^{hi}CD44^{hi} in B16F10 tumor bearing mice (FIG. 4F). Representative data from one of the three experiments performed in triplicate are shown as mean±s.e.m. Statistical analysis was performed by unpaired, one-tailed Student's t-test (***P≤0.001; ****P≤0.0001).

FIGS. 5A-5K: PARPi leads to metabolic reprograming and generation of T_{CM}^{sup} cells. PCA (gp100 vs gp100+olaparib) for different metabolites expression (FIG. 5A). Heat map analysis showing the top 50 differentially regulated metabolites in CD8 T cells from gp100 or gp100+olaparib group as shown (FIG. 5B). Heat map analysis for different metabolites implicated in TCA cycle (FIG. 5C). Heat map analysis showing different lipids in CD8 T cells from gp100 or gp100+olaparib group as shown (FIG. 5D). Metabolic characteristics of PARPi-treated CD8 T cells showing OCR and basal ECAR levels. Representative results from one of the two experiments performed in triplicate are shown (FIGS. 5E-5F). MFI of PGC1α and MitoFM in PARPi-induced T_{CM}^{sup} CD62L^{hi}CD44^{hi} compared to T_{CM} from gp100 (FIGS. 5G-5H). FACS analysis of variously in vitro-activated pMel-1 CD8⁺ T cells with or without PARPi or Sirt1 inhibitor for memory characteristics (T_{CM}; CD62L⁺CD44⁺). Percent of CD62L^{hi}CD44^{hi} from total T_{CM} in vitro-activated pMel-1 CD8⁺ T cells in the presence or absence of PARPi or Sirt1 inhibitor at 48 h of treatment (FIGS. 51-5K). The error bars indicate the s.e.m. Statistical analysis was performed by unpaired, one-tailed Student's t-test (*P≤0.05; **P≤0.001; ****P≤0.0001).

FIGS. 6A-6H: PARPi leads to trapping of the PARP1 protein resulting in a transcriptional switch, leading to metabolic reprograming and generation of T_{CM}^{sup} cells. PCA (gp100 vs gp100+olaparib) for different genes expression (FIG. 6A). Volcano plot showing differential gene expression in CD8 T cells from g100 or g100+olaparib at 48 h of treatment (FIG. 6B). Heat map analysis for different genes implicated in glycolysis, OXPHOS and FAO in CD8 T cells from g100 or g100+olaparib group at 48 h of treatment (FIG. 6C). Cpt1α and Acot1 mRNA expression relative to Actb in PARPi-induced T_{CM}^{sup} CD62L^{hi}CD44^{hi} compared to T_{CM} from gp100 (FIGS. 6D-6E). Western blot analysis using anti-PAR antibody in activated pMel-1 CD8 T cells in the presence or absence of PARPi at 48 h of treatment (FIG. 6F). Western blot analysis using anti-PARP1 antibody in activated pMel-1 CD8 T cells in presence or absence of PARPi at 48 h of treatment (FIG. 6G). FACS analysis of pMel-1 CD8 T cells activated with gp 100 in the presence of either olaparib or SiRNA for PARP1/2 or control SiRNA for memory characteristics at 48 h of various treatments (FIG. 6H).

FIGS. 7A-7F: PARPi mediated-T_{CM}^{sup} are generated through SIRT1/FOXO1 pathway activation. Heat map analysis for different genes implicated in FOXO1 pathway in activated pMel-1 CD8 T cells in the presence or absence of PARPi at 48 h of treatment (FIG. 7A). mRNA expression of Klf2, Tcf7, Eomes, and CD62L relative to Actb in PARPi-induced T_{CM}^{sup} CD62L^{hi}CD44^{hi} compared to T_{CM} from gp100 at 48 h of treatment (FIG. 7B). Hypothetic scheme for the Sirt1/FOXO1 signaling pathway after PARP inhibition (FIG. 7C). NAD accumulation measurement in activated pMel-1 CD8 T cells after PARP inhibition (FIG. 7D). Western blot analysis of activated pMel-1 CD8 T cells treated with or without PARPi using anti-SIRT-1, anti-Ac-FOXO1, anti-p-FOXO1, anti-FOXO1 (FIG. 7E). Immunofluorescence microscopy images of activated pMel-1 CD8 T cells using DAPI staining and anti-FOXO1 with secondary Alexa 488 antibody in activated pMel-1 CD8 T cells treated with or without PARPi at 48 h (FIG. 7F).

FIGS. 8A-8F: PARPi mediated-T_{CM}^{sup} are generated through SIRT1/FOXO1 pathway activation. FACS analysis for memory characteristics (T_{CM}; CD62L⁺CD44⁺) of in vitro-activated pMel-1 CD8⁺ T cells in the presence or absence of PARPi or SIRT1 inhibitor at 48 h of treatment (FIG. 8A). Percent of CD62L^{hi}CD44^{hi} from total T_{CM} CD8 T cells at 48 h of treatment (FIG. 8B). MFI of IFNγ in PARPi-induced CD62L^{hi}CD44^{hi} at 48 h or treatment (FIG. 8C). MFI of Gzm B PARPi-induced CD62L^{hi}CD44^{hi} at 48 h of treatment (FIG. 8D). FACS analysis for memory characteristics (T_{CM}; CD62L⁺CD44⁺) of in vitro-activated pMel-1 CD8⁺ T cells in the presence or absence of PARPi or FOXO1 inhibitor at 48 h of treatment (FIG. 8E). Percent of CD62L^{hi}CD44^{hi} from total T_{CM} CD8 T cells at 48 h of treatment (FIG. 8F) Representative data from one of two experiments shown as mean±s.e.m. Statistical analysis was performed by unpaired, one-tailed Student's t-test (*P≤0.05; **P≤0.01; ***P≤0.001; ****P≤0.0001).

FIGS. 9A-9E: PARPi mediated-T_{CM}^{sup} generation in human CD8 T cells through SIRT1/FOXO1 pathway activation. FACS analysis of in vitro-activated human CD8⁺ T cells for memory characteristics (T_{CM}, CD45RO⁺CCR7⁺ or CD45RO⁺CD62L⁺) in the presence or absence of PARP or SIRT1 inhibitors (FIGS. 9A-9B). Percent of CD45RO^{hi}CCR7^{hi} (FIG. 9C) or CD45RO^{hi}CD62L^{hi} (FIG. 9D) from total CD8 T cells after 48 h of treatment with PARP and/or SIRT1 inhibitors. MFI of IFNγ in human CD8 T cells after 48 h of treatment with PARP and/or SIRT1 inhibitors (FIG. 9D). Representative data from one of two experiments shown as mean±s.e.m. Statistical analysis was performed by unpaired, one-tailed Student's t-test (*P<0.05; **P<0.01; ***P<0.001; ****P<0.0001).

FIG. 10: Proposed model for the direct effect of PARP inhibition on CD8 T cells, leading to T_{CM}^{sup} generation. PARPi (1) inhibits the enzymatic activity of PARP and (2) results in the increased trapping of PARP in the nucleus. Inhibition of the enzymatic activity of PARP results in (3) NAD accumulation, (4) increased SIRT-1 activity, (5) increased SIRT-1-mediated PGC1-α expression, leading to (6) increased FAO and OXPHOS and decreased glycolysis, (7) increased SIRT-1-mediated accumulation of FOXO1 in the nucleus, (8) transcriptional modification, and in turn (9) increased expression of memory-related target genes of FOXO1. Furthermore, PARPi-induced (2) PARP trapping mediates (8) transcriptional modification that induces the expression of PARP target genes related to (9) memory phenotype and (10) FAO metabolism. Together, induction of these (11) metabolic and (8) transcriptional switches result in (12) the development of T_{CM}^{sup} cells that show (13) enhanced recall responses.

FIGS. 11A-11G: Experimental plan for tumor growth and survival in both B16F10 and TC-1 models (FIG. 11A). Tumor growth rate, mouse survival and SK plots in B16F10 tumor model after various treatments (FIG. 11B) (Untreated (UT); Vaccine (Vacc), PARPi, Vaccine plus PARPi (Vacc+PARPi)). Tumor growth and survival data are the average of three independent experiments. The error bars indicate the s.e.m. For tumor growth, statistical analysis was performed by unpaired, one-tailed Student's t-test. The P value in the tumor growth graph, *P≤0.05. Survival in various groups was compared using log-rank (Mantel-Cox) tests, **P≤0.01; ***P≤0.001; ****P≤0.0001. Tumor growth rate, in nude mice implanted with TC-1 or B16F10 cells (FIG. 11C). Analysis of immune infiltrate in tumors 2-3 days after second vaccination. Number of CD8⁺ and CD8⁺gp100⁺ T cells in B16F10 model (FIGS. 11D-11E). IFNγ⁺ CD8⁺ T and Gzm B⁺ CD8⁺ T cells in TC-1 model (FIGS. 11F-11G). One-tailed Student's t-test (*P≤0.05; **P≤0.001; ****P≤0.0001).

FIGS. 12A-12J: Analysis of immune infiltrate in tumors 2-3 days after second vaccination from various groups: Untreated (UT); Vaccine (Vacc), PARPi, Vaccine plus PARPi (Vacc+PARPi). Number of IFNγ⁺ CD8⁺ and Gzm B+CD8⁺ T cells in B16F10 model (FIGS. 12A-12B). Number of IFNγ⁺ gp100⁺CD8⁺ T and Gzm B+gp100 CD8⁺ T cells in B16F10 model (FIGS. 12C-12D). Tumor growth rate, mouse survival and SK plots in TC-1 model after CD8 depletion (FIG. 12E). Number of E7⁺CD8 T cells Effector memory in TC-1 model (FIG. 12F). CD8 effector memory cells in TC-1 model (FIG. 12G). Number of CD8 central memory cells in TC-1 model (FIG. 12H). CD8 central memory cells in B16F10 model (FIG. 12I). MFI Ki67 in CD8 T cells from B16F10 model (FIG. 12J).

FIGS. 13A-13F: Experimental plan of vitro-activated pMel-1 CD8⁺ T cells (FIG. 13A). Proliferation (by VCT dilution) histograms and percentage of each generation in vitro-activated pMel-1 CD8⁺ T cells with or without PARPi at 48 h (FIG. 13B). Histogram and percent of cell cycle phases in in vitro-activated pMel-1 CD8⁺ T cells with or without PARPi at 48 h population (FIG. 13C). MFI of IFNγ and Gzm B (FIGS. 13D-13E). Percentage of live cells at 48 h of treatment (FIG. 13F) in vitro-activated pMel-1 CD8⁺ T cells with or without PARPi at 48 h. One-tailed Student's t-test (*P≤0.05; **P≤0.001; ****P≤0.0001).

FIGS. 14A-14D: FACS analysis of in vitro-activated pMel-1 CD8⁺ T cells for memory characteristics (T_{CM}; CD62L⁺CD44⁺). Percent of CD62L^{hi}CD44^{hi} from total T_{CM} CD8 T in vitro-activated pMel-1 CD8⁺ T cells with or without PARPi at 48 h of treatment (FIG. 14A). Experimental plan for in vivo re-challenge, ratio of number of transferred cells at first and fourth vaccine (FIG. 14B). MFI of IFNγ and Gzm B in T_{CM} CD8⁺ T cells after re-challenge with different concentration of gp100 peptide for 24 h (FIG. 14C). Tumor growth rate in TC-1 model after active cell transfer of T_{CM} cells (CD62L⁺CD44⁺), CD62L^{int}CD44^{int} or CD62L^{hi}CD44^{hi} in B16F10 tumor bearing mice (FIG. 14D). Representative data from one of the three experiments performed in triplicate are shown as mean±s.e.m. Statistical analysis was performed by unpaired, one-tailed Student's t-test (***P≤0.001; ****P≤0.0001).

FIGS. 15A-15E: MFI of γH2AX in vitro-activated pMel-1 CD8⁺ T with or without PARPi (FIG. 15A). MFI of PARP1 (FIG. 15B) and MFI of PARP2 (FIG. 15C) in pMel-1 CD8 T cells activated with gp100 in the presence of either olaparib or SiRNA for PARP1/2 or control ScRNA. SIRT1 activity in vitro-activated pMel-1 CD8⁺ T with or without PARPi (FIG. 15D). Western blot analysis for nuclear fraction in activated pMel-1 CD8 T cells after PARPi using anti-FOXO1 (FIG. 15E).

### SEQUENCES

The amino acid sequences listed in the accompanying sequence listing are shown using single letter code for amino acids, as defined in 37 C.F.R. 1.822. In the accompanying sequence listing:
SEQ ID NO: 1 is the amino acid sequence of a TC-1-specific E7 peptide (RAHYNIVTF).
SEQ ID NO: 2 is the amino acid sequence of a B16-specific gp10025-33 nonamer peptide (KVPRNQDWL).
SEQ ID NO: 3 is the amino acid sequence of a pan-HLA DR-binding epitope (PADRE).
SEQ ID NO: 4 is the amino acid sequence of aK-Cha-VAAWTLKAAa, wherein “a” is D-alanine and “Cha” is 1-cyclohexylalanine.

### DETAILED DESCRIPTION

### I. Introduction

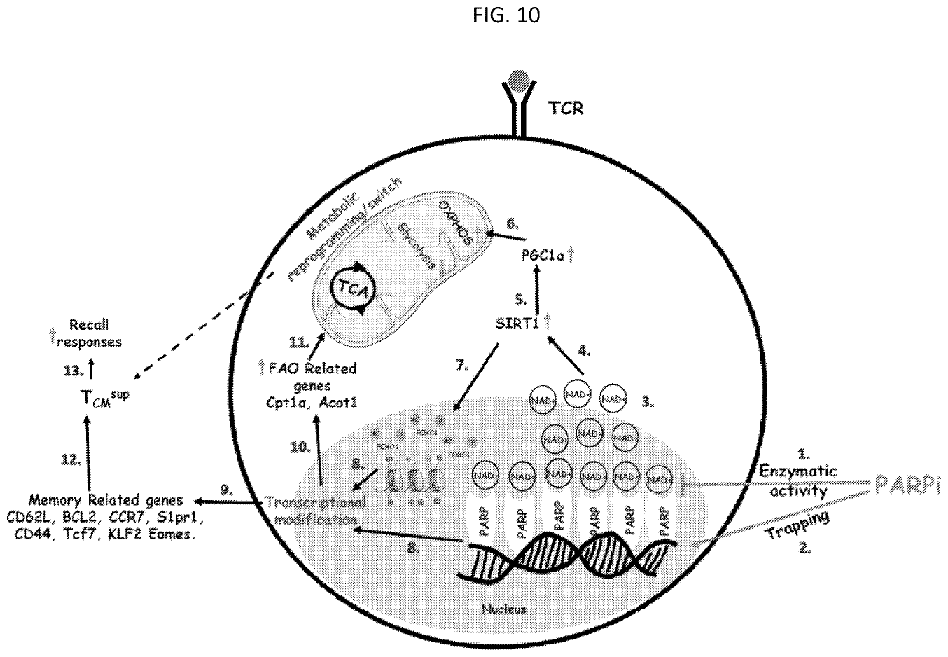

Less differentiated CD8 T cells including stem cell-like memory T cells (T_{SCM}) and central memory T cells (T_{CM}) provide superior anti-tumor immunity compared to their more-differentiated effector memory counterparts. It is disclosed herein that PARP inhibitor (PARPi) induces a unique subpopulation of superior central memory cells, T_{CM}^{sup} (also referred to herein as “PARPi-induced memory T cells”) that have significantly higher expression of memory markers, and higher recall responses than T_{CM}. T_{CM}^{sup} cells express both CD62L and CD44 (in mouse cells) and may express intermediate or high levels of these markers (CD62L^{int}CD44^{int} or CD62L^{hi}CD44^{hi}, respectively), corresponding to CD45RO and CD62L, or CD45RO and CCR7 in human cells. This is achieved by transcriptional and metabolic reprogramming of cells mediated by inhibition of PARP enzymatic activity and trapping of PARP in the nucleus. This results in the activation of the SIRT-1/FOXO1 pathway, and in turn leads to the upregulation of gene expression related to memory and fatty acid oxidation (FAO) in CD8 T cells (FIG. 10). This unique subpopulation was also generated after PARP inhibition in human CD8 T cells. This strategy of PARP inhibition generated CD8 T cells with higher efficacy for adoptive cell therapy. Moreover, PARPi treatment of tumor-bearing mice also showed a significantly enhanced immune-mediated anti-tumor effect, which was due to an increase in the number and cytotoxicity of antigen-specific CD8 T cells and the memory population within the TME. Together, these findings demonstrate the direct effect of PARP inhibition on CD8 T cells and its ability to modulate and reprogram these cells into metabolically fit T_{CM}^{sup} with enhanced recall capability that results in a greater anti-tumor immune response. Based on these findings, described herein are methods of producing T_{CM}^{sup}, such as for use in adoptive cell therapy applications, and for enhancing immunotherapies, increasing the potency of vaccines, and treating cancer.

### II

Abbreviations

| | |
|---|---|
| ACT | adoptive cell transfer |
| CAF | cancer associated fibroblast |
| CAR | chimeric antigen receptor |
| Cas9 | CRISPR-associated protein 9 |
| CRISPR | clustered regularly interspaced short palindromic repeats |
| DAG | diacylglycerol |
| DBQ | debrisoquine |
| DCER | dihydroceramide |
| DDR | DNA damage repair |
| ECAR | extracellular acidification rate |
| FACS | fluorescence activated cell sorting |
| FAO | fatty acid oxidation |
| FFA | free fatty acid |
| FOXO1 | forkhead box protein O1 |
| Gzm B | granzyme B |
| HCER | hexosylceramide |
| HLA | human leukocyte antigen |
| KO | knockout |
| MDSC | myeloid-derived suppressor cell |
| IL | interleukin |
| INFγ | interferon gamma |
| IU | international unit |
| LAL | lysosomal acid lipase |
| LCER | lactosylceramide |
| LC-MS | liquid chromatography-mass spectrometry |
| MFI | mean fluorescence intensity |
| NAD/NADH | nicotinamide adenine dinucleotide |
| OCR | oxygen consumption rate |
| OXPHOS | oxidative phosphorylation |
| PARP | poly(ADP-ribose) polymerase |
| PARPi | PARP inhibitor |
| PBMC | peripheral blood mononuclear cell |
| PCA | principal component analysis |
| PGC1α | peroxisome proliferator-activated receptor gamma |
| | coactivator-1 alpha |
| rRNA | ribosomal RNA |
| scRNA | scrambled control RNA |
| s.e.m. | standard error of the mean |
| SSB | single-strand break |
| siRNA | small inhibitory RNA |
| SIRT1 | sirtuin 1 |
| TAG | triacylglyceride |
| TCA | tricarboxylic acid |
| TCR | T cell receptor |
| TME | tumor microenvironment |
| T_{CM} | central memory T cells |
| T_{SCM} | stem cell-like memory T cells |
| T_{CM}^{sup} | supercentral memory cells |
| WT | wild-type |

### III. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administration: To provide or give a subject an agent, such as a PARP inhibitor, by any effective route. Exemplary routes of administration include, but are not limited to, oral, injection (such as subcutaneous, intramuscular, intradermal, intraperitoneal, intravenous, intraprostatic, and intratumoral), sublingual, rectal, transdermal, intranasal, vaginal and inhalation routes.

Antibody: A polypeptide ligand comprising at least one variable region that recognizes and binds (such as specifically recognizes and specifically binds) an epitope of an antigen, such as a tumor antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles and is functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., JMB 273,927-948, 1997; the “Chothia” numbering scheme), Kunik et al. (see Kunik et al., PLoS Comput Biol 8: e1002388, 2012; and Kunik et al., Nucleic Acids Res 40 (Web Server issue): W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online. In addition, the AbRSA tool can be used to determine the CDR boundaries according to Kabat, IMGT or Chothia (online at aligncdr.labshare.cn/aligncdr/abrsa.php).

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid VAH antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by methods known to those of skill in the art. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they must be substantially identical to human immunoglobulin constant regions, such as at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Allogeneic: Refers to individuals, tissues or cells that are of the same species, but are antigenically distinct.

Autologous: Derived from the same individual. In the context of the present disclosure, “autologous” CD8+ T used for treatment of a subject are CD8+ T cells originally obtained from the subject.

Cancer, tumor, or malignancy: An abnormal growth of tissue or cells which results from excessive cell division. Neoplastic growth can produce a tumor. The amount of a tumor in an individual is the “tumor burden” which can be measured as the number, volume, or weight of the tumor. A tumor that invades the surrounding tissue and/or can metastasize to a location distant from the original/primary tumor is referred to as “malignant.”

Exemplary tumors, such as cancers, include but are not limited to breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, liver cancer, (such as hepatocellular carcinoma or hepatoblastoma), bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck cancers, and lymphatic tumors (including B-cell and T-cell malignant lymphoma). In some examples, the cancer is a melanoma or a cervical cancer.

Chimeric antigen receptor (CAR): A chimeric molecule that includes an antigen-binding portion (such as a scFv antibody fragment) and a signaling domain, such as a signaling domain from a T cell receptor (for example, CD32). Typically, CARs are comprised of an antigen-binding moiety, a transmembrane domain and an endodomain. The endodomain typically includes a signaling chain having an immunoreceptor tyrosine-based activation motif (ITAM), such as CD3ζ or FcεRIγ. In some instances, the endodomain further includes the intracellular portion of at least one additional co-stimulatory domain, such as CD28, 4-1BB (CD137), ICOS, OX40 (CD134), CD27 and/or DAP10. In some examples, the CAR is multispecific (such as bispecific) or bicistronic.

Contacting: Placement in direct physical association; includes both in solid and liquid form.

Immune response: A response of a cell of the immune system, such as a B cell, T cell, or monocyte, to a stimulus. In some aspects, the immune response is a T cell response, such as a CD4+ T cell response or a CD8+ T cell response. In other aspects, the response is a B cell response, and results in the production of specific antibodies.

Immunotherapy: Any type of therapy or treatment that utilizes the immune system to recognize and attack cancer cells. Exemplary types of immunotherapy include monoclonal antibodies targeted to a tumor antigen, immune cells (such as T cells) expressing tumor-targeted chimeric antigen receptors (CARs), cancer vaccines, checkpoint inhibitors, costimulatory agonist antibodies, oncolytic viruses, and cytokines, such as interferon or IL-2.

Niraparib: A small molecule PARP inhibitor. Also known as MK-4827 and ZEJULA (GlaxoSmithKline).

Olaparib: A small molecule inhibitor of PARP. Olaparib selectively binds and inhibits PARP, inhibiting PARP-mediated repair of single strand DNA breaks. Also known as AZD2281 and LYNPARZA (AstraZeneca).

PARP inhibitor (PARPi): Any agent, compound or molecule that reduces, inhibits, or eliminates expression or activity of PARP, such as PARP-1, PARP-2, or both. In some aspects herein, the PARPi is a small molecule inhibitor, such as, but not limited to, olaparib (AZD-2281), niraparib, talazoparib (BMN-673), rucaparib, iniparib (BSI-201), saruparib (AZD 5305), veliparib (ABT-888), AG014699, CEP9722, CEP8983, MK4827, INO-1001, E7016, AZD2461, LT-673, PD128763, BGB-290, and 3-aminobenzamide (see, e.g., Rouleau et al., Nat Rev Cancer 10:293-301, 2010; Underhill et al., Ann Oncol 22 (2): 268-279, 2011).

Pharmaceutically acceptable carriers: The pharmaceutically acceptable carriers of use are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of the antibodies and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Poly(ADP-ribose) polymerase (PARP): An enzyme that catalyzes the transfer of ADP-ribose to target proteins. PARP plays an important role in several cellular processes, including modulation of chromatin structure, transcription, replication, recombinant, and DNA repair (Morales et al., Crit Rev Eukaryot Gene Expr 24 (1): 15-28, 2014). In the context of the present disclosure, “PARP” refers to PARP1 (NCBI Gene ID 142), PARP2 (NCBI Gene ID 10038), or both.

Small molecule: A molecule, typically with a molecular weight less than about 1000 Daltons, or in some aspects, less than about 500 Daltons, wherein the molecule is capable of modulating, to some measurable extent, an activity of a target molecule (e.g., PARP).

Subject: Living multi-cellular vertebrate organisms, a category that includes both human and veterinary subjects, including human and non-human mammals.

Super central memory CD8+ T cells (T_{CM}^{sup}): As used herein, a “super central memory CD8+ T cell” is a unique subset of central memory T cells that expresses both CD62L and CD44 (such as CD62L^{int}CD44^{int} or CD62L^{hi}CD44^{hi}). CD62L and CD44 are the memory markers in mouse cells, but these markers correspond to CD45RO and CD62L, or CD45RO and CCR7 in human cells. Thus, T_{CM}^{sup} cells include cells positive for expression of CD45RO and CD62L (such as CD45RO^{int} and CD62L^{int} or CD45RO^{hi} and CD62L^{hi}), or CD45RO and CCR7 (such as CD45RO^{int} and CCR7^{int} or CD45RO^{hi} and CCR7^{hi}). These cells are also referred to herein as “PARPi-induced memory T cells.” T_{CM}^{sup} cells exhibit significantly higher expression of memory markers, less proliferative capacity, and higher recall responses than T_{CM}.

T cells: A white blood cell that is a component of the immune system. Also known as a T lymphocyte. T cells are involved in preventing infections and the development of cancer. A central memory T cell (T_{CM}) refers to a type of antigen-specific memory T cell characterized by expression of CD45RO, CCR7 and CD62L for human T cells. Central memory T cells are commonly found in the lymph nodes and the peripheral circulation.

Talazoparib: A small molecule inhibitor of PARP. Also known as BMN-673 and TALZENNA (Pfizer).

Therapeutically effective amount: A quantity of a specific substance sufficient to achieve a desired effect in a subject being treated. For instance, this can be the amount necessary to inhibit or suppress growth of a tumor. In one aspect, a therapeutically effective amount is the amount necessary to eliminate, reduce the size, or prevent metastasis of a tumor, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment. When administered to a subject, a dosage will generally be used that will achieve target tissue concentrations (for example, in tumors) that has been shown to achieve a desired in vitro effect.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition, such as cancer. A vaccine may include a polynucleotide (such as a nucleic acid encoding a tumor antigen or viral antigen), a peptide or polypeptide (such as a tumor antigen or a viral antigen), a virus, a cell or one or more cellular constituents.

Veliparib: A small molecule PARP inhibitor. Also known as ABT-888 (AbbVie),

### IV. Methods of Producing and Using Super Central Memory CD8 T Cells

It is disclosed herein that inhibiting poly(ADP-ribose) polymerase (PARP) in T cells induces a novel CD8 T cell central memory subtype that is highly potent with unique characteristics. These cells are referred to herein as super-central memory CD8 T cells (T_{CM}^{sup}) or PARPi-induced memory T cells. These cells are significantly more efficient for cancer therapy than traditional central memory T cells (T_{CM}) generated by tumor antigens. Accordingly, these findings demonstrate that pharmacological inhibition of PARP and thereby its trapping in the nucleus can be used as an immune modulating drug to either (1) enhance immunotherapy drugs and/or vaccines in vivo; or (2) enhance the potency of T cells ex vivo for adoptive T cell therapy (ACT). For the ex vivo application, CD8 T cells can be obtained either from the cancer patients (autologous) or they can be allogeneic. PARP is a key molecule in the repair of DNA single-strand breaks (SSBs). Both PARP-1 and PARP-2 use (NAD)+ as a cofactor in their catalytic activity, where continuous activation of PARP reduces intracellular levels of (NAD)+ by 80%. Inhibition of PARP was used as a therapeutic strategy against tumors associated with deficiency or mutation in DNA damage repair (DDR) genes by inducing synthetic lethality. However, innate and adaptive resistance in patients with DDR gene mutations were reported after treatment with PARP inhibitor (PARPi), highlighting the potential implication of other compartments such as the role of immune cells in the mechanism of resistance.

Importantly, PARPi is demonstrated herein to have an immune-modulatory capacity within the tumor microenvironment (TME) by activating the STING pathway and increasing INF-γ and chemokine secretion and hence increasing the recruitment and function of CD8 T cells. In addition, PARPi is shown to affect other immune cells within the TME, such as macrophages, myeloid-derived suppressor cells (MDSCs) and cancer associated fibroblasts (CAFs), promoting either an immune enhancement or immune suppressor milieu that could indirectly affect CD8 T cell function. However, little is known about the direct effect of PARPi on CD8 T cells. Hence, the present disclosure describes an investigation of the effect of PARPi on CD8 T cell phenotype and functionality using PARP inhibitors (e.g., olaparib, niraparib, and talazoparib) at the time of priming in T cells. It was found that PARPi controls the tumor growth and increases survival in tumor-bearing mice. This effect was due to enhancement of the number and cytotoxicity of antigen-specific CD8 T cells and the maintenance of the memory population within the TME. In vitro results showed that PARPi significantly increased the percentage of a population with high expression of CD62L and CD44 (CD44^{hi}CD62L^{hi}) that had higher expression of IFN-γ and granzyme B compared to T_{CM} from the gp100-treated group and the intermediate population (CD44^{int}CD62L^{int}) from the PAPRi group. These CD44^{hi}CD62L^{hi} memory CD8 T cells also showed a stronger recall response compared to the other T_{CM} population (CD44^{int}CD62L^{int}) in PARPi and gp100-treated groups based on the INF-γ and granzyme B expression. When the anti-tumor effect of these three populations of CD8 T cells described above were assessed after their ACT into C57BL/6 B16F10 tumor-bearing mice, it was found that mice receiving the PARPi-induced CD44^{hi}CD62L^{hi} T_{CM} had smaller tumor size compared to mice that either received PARPi-induced CD44^{int}CD62L^{int} cells or gp100-induced T_{CM} cells. These results demonstrated the greater anti-tumor effect of super-central memory CD8 T cells.

It is also disclosed herein that PARPi enhances the metabolic pathways associated with memory CD8 T cells, resulting in metabolic reprograming that supports the generation of metabolically fit T_{CM}^{sup} cells with enhanced recall activity that also survived longer. The mechanism of induction of these cells was also determined. PARPi activates the SIRT-1/FOXO1 pathway, leading to the modification of gene expression related to memory and fatty acid oxidation (FAO) metabolism in CD8 T cells. As a result, a unique subpopulation of superior central memory cells with high recall responses and anti-tumor effects are generated after PARPi treatment. These findings highlight the direct effect of PARP inhibition on CD8 T cells and its ability to modulate and reprogram CD8 T cells within the TME, leading to a greater anti-tumor immune response. It is also demonstrated that PARPi activates the same pathway in human CD8 T cells to generate T_{CM}^{sup} CD8 T cells.

### A. Methods of Producing Super Central Memory CD8⁺ T Cells

Provided herein are ex vivo methods of producing super central memory CD8+ T cells that express CD62L and CD44 (or CD45RO and CD62L, or CD45RO and CCR7, for human cells). In some aspects, the method includes culturing CD8+ T cells in the presence of a poly(ADP-ribose) polymerase (PARP) inhibitor (PARPi), thereby generating super central memory CD8+ T cells that express CD62L and CD44, CD45RO and CD62L, or CD45RO and CCR7. The CD8+ T cells can be obtained or isolated from a variety of different sources or samples. In some examples, the CD8+ T cells are peripheral blood CD8+ T cells, tumor-infiltrating CD8+ T cells, or engineered CD8+ T cells.

In some aspects, the super central memory CD8+ T cells express intermediate levels of CD62L and CD44 (CD62L^{int}CD44^{int}), intermediate levels of CD45RO and CD62L (CD45RO^{int} and CD62L^{int}), or intermediate levels of CD45RO and CCR7 (CD45RO^{int} and CCR7^{int}). In other aspects, the super central memory CD8+ T cells express high levels of CD62L and CD44 (CD62L^{hi}CD44^{hi}), high levels of CD45RO and CD62L (CD45RO^{hi} and CD62L^{hi}), or high levels of CD45RO and CCR7 (CD45RO^{hi} and CCR7^{hi}).

In some aspects, the PARPi is a small molecule inhibitor. The PARPi can be an inhibitor of PARP-1, PARP-2, or both. In some examples, the small molecule PARPi includes olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, 3-aminobenzamide, or any combination thereof (such as any combination of two, three, four, or five PARP inhibitors).

In some aspects of the ex vivo method, the CD8+ T cells are cultured in the presence of about 1 nM to about 15 μM of the small molecule inhibitor. In some examples, the CD8+ T cells are cultured in the presence of about 5 nM to about 14 μM, about 10 nM to about 13 μM, about 15 nM to about 12 μM, about 20 nM to about 11 μM, about 25 nM to about 10 μM, about 50 nM to about 10 μM, about 100 nM to about μM, about 150 nM to about 10 μM, about 200 nM to about 10 μM, about 250 nM to about 5 μM, about 500 nM to about 5 μM, about 750 nM to about 5 μM, about 1000 nM to about 5 μM, about 1 μM to about 5 μM, about 2 μM to about 5 μM, or about 5 μM to about 10 μM. In particular examples, the CD8+ T cells are cultured in the presence of about 1 nM, about 5 nM, about 10 nM, about 15 nM, about 20 nM, about 25 nM, about 30 nM, about 40 nM, about 50 nM, about 75 nM, about 100 nM, about 125 nM, about 150 nM, about 175 nM, about 200 nM, about 250 nM, about 400 nM, about 500 nM, about 750 nM, about 1000 nM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM.

In some aspects, the CD8+ T cells are cultured in the presence of the small molecule PARP inhibitor for at least 3 hours, at least 6 hours, at least 9 hours, at least 12 hours, at least 24 hours, at least 36 hours, at least 48 hours, about 60 hours, or at least 72 hours. In some examples, the CD8+ T cells are cultured in the presence of the small molecule PARP inhibitor for about 6 hours, about 12 hours, about 18 hours, about 24 hours, about 30 hours, about 36 hours, about 42 hours, about 48 hours, about 54 hours, about 60 hours, about 66 hours or about 72 hours.

In some aspects, the CD8+ T cells are obtained from a subject with cancer, such as a subject that is to be treated with the super central memory CD8+ T cells. In other aspects, the CD8+ T cells are obtained from a healthy subject (e.g., a subject who does not have cancer). In some examples, the CD8+ T cells are part of or isolated from a peripheral blood sample obtained from the subject. In particular examples, the CD8+ T cells are tumor-infiltrating T cells, or engineered T cells.

B. Methods of Treating Cancer by Adoptive Transfer of Super Central Memory CD8+ T Cells Also provided herein are methods of treating cancer by adoptive transfer of super central memory CD8+ T cells. In some aspects, the method includes culturing CD8+ T cells ex vivo with a PARP inhibitor (PARPi), thereby producing super central memory CD8+ T cells that express CD62L and CD44, CD45RO and CD62L, or CD45RO and CCR7, and adoptively transferring the super central memory CD8+ T cells to the subject.

In some aspects, the CD8+ T cells are autologous CD8+ T cells obtained from the subject. In other aspects, the CD8+ T cells are allogeneic CD8+ T cells. In some examples, the allogeneic CD8+ T cells are obtained from a healthy donor.

In some aspects, the CD8+ T cells are peripheral blood CD8+ T cells, tumor-infiltrating CD8+ T cells, or engineered CD8+ T cells.

In some aspects, the super central memory CD8+ T cells express intermediate levels of CD62L and CD44 (CD62L^{int}CD44^{int}), intermediate levels of CD45RO and CD62L (CD45RO^{int} and CD62L^{int}), or intermediate levels of CD45RO and CCR7 (CD45RO^{int} and CCR7^{int}). In other aspects, the super central memory CD8+ T cells express high levels of CD62L and CD44 (CD62L^{hi}CD44^{hi}), high levels of CD45RO and CD62L (CD45RO^{hi} and CD62L^{hi}), or high levels of CD45RO and CCR7 (CD45RO^{hi} and CCR7^{hi}).

In some aspects, the PARPi is a small molecule inhibitor, such as a small molecule that inhibits PARP-1, PARP-2, or both. In some examples, the PARPi includes olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, 3-aminobenzamide, or any combination thereof (such as any combination of two, three, four, or five PARP inhibitors).

In some aspects of the method, the CD8+ T cells are cultured in the presence of about 1 nM to about 15 μM of the small molecule inhibitor. In some examples, the CD8+ T cells are cultured in the presence of about 5 nM to about 14 μM, about 10 nM to about 13 μM, about 15 nM to about 12 μM, about 20 nM to about 11 μM, about 25 nM to about 10 μM, about 50 nM to about 10 μM, about 100 nM to about μM, about 150 nM to about 10 μM, about 200 nM to about 10 μM, about 250 nM to about 5 μM, about 500 nM to about 5 μM, about 750 nM to about 5 μM, about 1000 nM to about 5 μM, about 1 μM to about 5 μM, about 2 μM to about 5 μM, or about 5 μM to about 10 μM. In particular examples, the CD8+ T cells are cultured in the presence of about 1 nM, about 5 nM, about 10 nM, about 15 nM, about 20 nM, about 25 nM, about 30 nM, about 40 nM, about 50 nM, about 75 nM, about 100 nM, about 125 nM, about 150 nM, about 175 nM, about 200 nM, about 250 nM, about 400 nM, about 500 nM, about 750 nM, about 1000 nM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM.

In some aspects, the CD8+ T cells are cultured in the presence of the small molecule PARP inhibitor for at least 3 hours, at least 6 hours, at least 9 hours, at least 12 hours, at least 24 hours, at least 36 hours, at least 48 hours, about 60 hours, or at least 72 hours. In some examples, the CD8+ T cells are cultured in the presence of the small molecule PARP inhibitor for about 6 hours, about 12 hours, about 18 hours, about 24 hours, about 30 hours, about 36 hours, about 42 hours, about 48 hours, about 54 hours, about 60 hours, about 66 hours or about 72 hours.

In some aspects of the treatment method, the subject is further administered an immunotherapy for treating the cancer. The subject can be treated with any type of immunotherapy, and treatment can be administered either before adoptive transfer of the super central memory CD8+ T cells, concurrent with adoptive transfer of the super central memory CD8+ T cells, or following adoptive transfer of the super central memory CD8+ T cells (or any combination thereof).

In some examples, the immunotherapy includes administration of a monoclonal antibody, such as a monoclonal antibody that specifically targets a tumor antigen. In some examples, the monoclonal antibody is an anti-VEGF (e.g., bevacizumab), anti-FGFR4, anti-mesothelin, anti-Her2/neu, anti-CD38, anti-CCR4, anti-CD19, anti-CD20, anti-VEGFR2, anti-CD30, anti-CD33, or anti-CD22 antibody (see, e.g., Zahavi and Weiner, Antibodies (Basel) 9 (3): 34, 2020, for examples of monoclonal antibodies that can be used for cancer immunotherapy).

In other examples, the immunotherapy includes immune cells (such as T cells, B cells, macrophages, dendritic cells or induced pluripotent stem cells) expressing tumor-targeted chimeric antigen receptors (CARs). For example, the CARs can include a monoclonal antibody or antigen-binding fragment of a monoclonal antibody that specifically binds a tumor antigen. CAR T cell (or CAR immune cell) targets can include, for example, CD123, CLL-1, CD19, CD7, CD33, CD38, CD70, FLT3, B7-H3 (CD276), CD64, TIM-3, and CD13 (see, e.g., Schorr and Perna, Front Immunol 13:1085978, 2022).

In other examples, the immunotherapy includes administration of an immune checkpoint inhibitor. In specific examples, the checkpoint inhibitor is an agent that inhibits CTLA-4 (e.g., ipilimumab), PD-1 (e.g., pembrolizumab or nivolumab), PD-L1 (e.g., atezolizumab, avelumab or durvalumab), or a costimulatory agonist antibody, such as an OX40 or GITR antibody (see, e.g., Shiravand et al., Curr Oncol 29 (5): 3044-3060, 2022).

In other examples, the immunotherapy includes administration of an oncolytic virus. In particular examples, the oncolytic virus is talimogene laherparepvec (T-VEC; LMLYGIC®), for the treatment of metastatic melanoma, or Teserpaturev, for the treatment of malignant glioma.

In other examples, the immunotherapy includes administration of a cancer vaccine. The cancer vaccine can be any one of a number of different types, such as a protein/peptide vaccine, a DNA or RNA vaccine, a whole cell vaccine, a dendritic cell vaccine, or a virus vaccine (see, e.g., Maeng and Berzofsky, F1000Res 2019 May 13; 8: F1000 Faculty Rev-654; Terbuch and Lopez, Vaccines (Basel) 6 (3): 52, 2018; and Lopes et al., J Exp Clin Cancer Res 38 (1): 146, 2019). In some examples, the cancer vaccine is Sipuleucel-T (PROVENGB®), Nadofaragene firadonevec (ADSTILADRIN®), or T-VEC (LMLYGIC®).

In other examples, the immunotherapy includes administration of a cytokine. In some examples, the cytokine is an interferon (IFN), such as IFN-α. In some examples, the cytokine is interleukin-2 (IL-2).

The methods disclosed herein can be used to treat any type of cancer, such as any solid tumor. Exemplary cancers include, but are not limited to, breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck cancers, and lymphatic tumors (including B-cell and T-cell malignant lymphoma). In some examples, the cancer is a melanoma or a cervical cancer.

### C. Methods of Enhancing an Immune Response to a Vaccine

Further provided herein are methods of enhancing an immune response to a vaccine in a subject. In some aspects, the method includes administering to the subject the vaccine and a therapeutically effective amount of a PARP inhibitor (PARPi).

In some aspects, the PARPi is a small molecule inhibitor, such as a small molecule that inhibits PARP-1, PARP-2, or both. In some examples, the PARPi includes olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, 3-aminobenzamide, or any combination thereof (such as any combination of two, three, four, or five PARP inhibitors).

In some aspects, the therapeutically effective amount of the PARPi is about 2.5 mg/kg to about 5.0 mg/kg, such as about 3.0 mg/kg to about 4.5 mg/kg or about 3.5 mg/kg to about 4 mg/kg. In some examples, the therapeutically effective amount of the PARPi is about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg.

In some aspects, the therapeutically effective amount of the PARPi is about 200 mg to about 300 mg per day, such as about 200 mg to about 275 mg, about 200 mg to about 250 mg, about 200 mg to about 275 mg, about 225 to about 300 mg, about 250 mg to about 300 mg, about 275 mg to about 300 mg, or about 225 mg to about 275 mg per day. In some examples, the therapeutically effective amount of the PARPi is about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg per day.

In other aspects, the method includes administering to the subject the vaccine and a therapeutically effective amount of super central memory CD8⁺ T cells that CD62L and CD44, CD45RO and CD62L, or CD45RO and CCR7.

In some aspects, the method further includes producing the super central memory CD8+ T cells that express CD62L and CD44, CD45RO and CD62L, or CD45RO and CCR7 by culturing CD8+ T cells with a PARPi.

In some aspects, the CD8+ T cells are autologous CD8+ T cells obtained from the subject. In other aspects, the CD8+ T cells are allogeneic CD8+ T cells. In some examples, the allogeneic CD8+ T cells are obtained from a healthy donor.

In some aspects, the CD8+ T cells are peripheral blood CD8+ T cells, tumor-infiltrating CD8+ T cells, or engineered CD8+ T cells.

In some aspects, the super central memory CD8+ T cells express intermediate levels of CD62L and

CD44 (CD62L^{int}CD44^{int}), intermediate levels of CD45RO and CD62L (CD45RO^{int} and CD62L^{int}), or intermediate levels of CD45RO and CCR7 (CD45RO^{int} and CCR7^{int}). In other aspects, the super central memory CD8⁺ T cells express high levels of CD62L and CD44 (CD62L^{hi}CD44^{hi}), high levels of CD45RO and CD62L (CD45RO^{hi} and CD62L^{hi}), or high levels of CD45RO and CCR7 (CD45RO^{hi} and CCR7^{hi}).

In some aspects, the PARPi is a small molecule inhibitor, such as a small molecule that inhibits PARP-1, PARP-2, or both. In some examples, the PARPi includes olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, 3-aminobenzamide, or any combination thereof (such as any combination of two, three, four, or five PARP inhibitors).

In some aspects of the method, the CD8+ T cells are cultured in the presence of about 1 nM to about 15 μM of the small molecule inhibitor. In some examples, the CD8+ T cells are cultured in the presence of about 5 nM to about 14 μM, about 10 nM to about 13 μM, about 15 nM to about 12 μM, about 20 nM to about 11 μM, about 25 nM to about 10 μM, about 50 nM to about 10 μM, about 100 nM to about μM, about 150 nM to about 10 μM, about 200 nM to about 10 μM, about 250 nM to about 5 μM, about 500 nM to about 5 μM, about 750 nM to about 5 μM, about 1000 nM to about 5 μM, about 1 μM to about 5 μM, about 2 μM to about 5 μM, or about 5 μM to about 10 μM. In particular examples, the CD8+ T cells are cultured in the presence of about 1 nM, about 5 nM, about 10 nM, about 15 nM, about 20 nM, about 25 nM, about 30 nM, about 40 nM, about 50 nM, about 75 nM, about 100 nM, about 125 nM, about 150 nM, about 175 nM, about 200 nM, about 250 nM, about 400 nM, about 500 nM, about 750 nM, about 1000 nM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM.

In some aspects, the CD8+ T cells are cultured in the presence of the small molecule PARP inhibitor for at least 3 hours, at least 6 hours, at least 9 hours, at least 12 hours, at least 24 hours, at least 36 hours, at least 48 hours, about 60 hours, or at least 72 hours. In some examples, the CD8+ T cells are cultured in the presence of the small molecule PARP inhibitor for about 6 hours, about 12 hours, about 18 hours, about 24 hours, about 30 hours, about 36 hours, about 42 hours, about 48 hours, about 54 hours, about 60 hours, about 66 hours or about 72 hours.

In some aspects of the method, the therapeutically effective amount of the super central memory CD8+ T cells that express CD62L and CD44, CD45RO and CD62L, or CD45RO and CCR7 is about 1 to about 10 million T cells/kg, such as about 2 to about 9 million T cells/kg, about 3 to about 8 million T cells/kg, about 4 to about 7 million T cells/kg, or about 5 to about 6 million T cells/kg. In some examples, the therapeutically effective amount of the super central memory CD8+ T cells that express CD62L and CD44, CD45RO and CD62L, or CD45RO and CCR7 is about 1 million T cells/kg, about 1.5 million T cells/kg, about 2 million T cells/kg, about 2.5 million T cells/kg, about 3 million T cells/kg, about 3.5 million T cells/kg, about 4 million T cells/kg, about 4.5 million T cells/kg, about 5 million T cells/kg, about 5.5 million T cells/kg, about 6 million T cells/kg, about 6.5 million T cells/kg, about 7 million T cells/kg, about 7.5 million T cells/kg, about 8 million T cells/kg, about 8.5 million T cells/kg, about 9 million T cells/kg, about 9.5 million T cells/kg, or about 10 million T cells/kg.

In some aspects of the disclosed methods, the vaccine is a cancer vaccine. The cancer vaccine can be any one of a number of different types, such as a protein/peptide vaccine, a DNA or RNA vaccine, a whole cell vaccine, a dendritic cell vaccine, or a virus vaccine (see, e.g., Maeng and Berzofsky, F1000Res 2019 May 13:8:F1000 Faculty Rev-654; Terbuch and Lopez, Vaccines (Basel) 6 (3): 52, 2018; and Lopes et al., J Exp Clin Cancer Res 38 (1): 146, 2019). In some examples, the cancer vaccine is Sipuleucel-T (PROVENGE®), Nadofaragene firadonevec (ADSTILADRIN®), or T-VEC (LMLYGIC®).

In other aspects of the disclosed methods, the vaccine is a viral or a bacterial vaccine. Examples of virus vaccines include, but are not limited to, vaccines against influenza virus, severe acute respiratory syndrome coronavirus-2 (SARS-COV-2), respiratory syncytial virus, measles virus, mumps virus, rubella virus, hepatitis A virus, hepatitis B virus, poliovirus, rabies virus, rotavirus, varicella virus, smallpox virus, human papillomavirus, Japanese encephalitis virus, yellow fever virus, dengue virus, and West Nile virus. Examples of bacterial vaccines include, but are not limited to, vaccines against cholera, diphtheria, anthrax, Haemophilus influenzae type b (Hib), meningococcal infection, pertussis, pneumococcal infection, tetanus, and tuberculosis.

D. Methods of Treating Cancer Using PARPi in Combination with Immunotherapy

Also provided herein are methods of treating cancer that include a combination of an immunotherapy and either treatment with a PARPi or treatment with PARPi-induced central memory T cells.

In some aspects, the method includes administering to the subject an immunotherapy and a therapeutically effective amount of a PARP inhibitor (PARPi), thereby treating the cancer in the subject.

In some aspects, the PARPi is a small molecule inhibitor, such as a small molecule that inhibits PARP-1, PARP-2, or both. In some examples, the PARPi includes olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, 3-aminobenzamide, or any combination thereof (such as any combination of two, three, four, or five PARP inhibitors).

In some aspects, the therapeutically effective amount of the PARPi is about 2.5 mg/kg to about 5.0 mg/kg, such as about 3.0 mg/kg to about 4.5 mg/kg or about 3.5 mg/kg to about 4 mg/kg. In some examples, the therapeutically effective amount of the PARPi is about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg.

In some aspects, the therapeutically effective amount of the PARPi is about 200 mg to about 300 mg per day, such as about 200 mg to about 275 mg, about 200 mg to about 250 mg, about 200 mg to about 275 mg, about 225 to about 300 mg, about 250 mg to about 300 mg, about 275 mg to about 300 mg, or about 225 mg to about 275 mg per day. In some examples, the therapeutically effective amount of the PARPi is about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg per day.

In other aspects of the methods, the method includes administering to the subject an immunotherapy and a therapeutically effective amount of super central memory CD8+ T cells expressing CD62L and CD44, CD45RO and CD62L, or CD45RO and CCR7.

In some aspects, the method further includes producing the super central memory CD8+ T cells that express CD62L and CD44, CD45RO and CD62L, or CD45RO and CCR7 by culturing CD8+ T cells with a PARPi.

In some aspects, the CD8+ T cells are autologous CD8+ T cells obtained from the subject. In other aspects, the CD8+ T cells are allogeneic CD8+ T cells. In some examples, the allogeneic CD8+ T cells are obtained from a healthy donor.

In some aspects, the CD8+ T cells are peripheral blood CD8+ T cells, tumor-infiltrating CD8+ T cells, or engineered CD8+ T cells.

In some aspects, the super central memory CD8+ T cells express intermediate levels of CD62L and CD44 (CD62L^{int}CD44^{int}), intermediate levels of CD45RO and CD62L (CD45RO^{int} and CD62L^{int}), or intermediate levels of CD45RO and CCR7 (CD45RO^{int} and CCR7^{int}). In other aspects, the super central memory CD8+ T cells express high levels of CD62L and CD44 (CD62L^{hi}CD44^{hi}), high levels of CD45RO and CD62L (CD45RO^{hi} and CD62L^{hi}), or high levels of CD45RO and CCR7 (CD45RO^{hi} and CCR7^{hi}).

In some aspects, the PARPi is a small molecule inhibitor, such as a small molecule that inhibits PARP-1, PARP-2, or both. In some examples, the PARPi includes olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, 3-aminobenzamide, or any combination thereof (such as any combination of two, three, four, or five PARP inhibitors).

In some aspects of the method, the CD8+ T cells are cultured in the presence of about 1 nM to about 15 μM of the small molecule inhibitor. In some examples, the CD8+ T cells are cultured in the presence of about 5 nM to about 14 μM, about 10 nM to about 13 μM, about 15 nM to about 12 μM, about 20 nM to about 11 μM, about 25 nM to about 10 μM, about 50 nM to about 10 μM, about 100 nM to about μM, about 150 nM to about 10 μM, about 200 nM to about 10 μM, about 250 nM to about 5 μM, about 500 nM to about 5 μM, about 750 nM to about 5 μM, about 1000 nM to about 5 μM, about 1 μM to about 5 μM, about 2 μM to about 5 μM, or about 5 μM to about 10 μM. In particular examples, the CD8+ T cells are cultured in the presence of about 1 nM, about 5 nM, about 10 nM, about 15 nM, about 20 nM, about 25 nM, about 30 nM, about 40 nM, about 50 nM, about 75 nM, about 100 nM, about 125 nM, about 150 nM, about 175 nM, about 200 nM, about 250 nM, about 400 nM, about 500 nM, about 750 nM, about 1000 nM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM.

In some aspects, the CD8+ T cells are cultured in the presence of the small molecule PARP inhibitor for at least 3 hours, at least 6 hours, at least 9 hours, at least 12 hours, at least 24 hours, at least 36 hours, at least 48 hours, about 60 hours, or at least 72 hours. In some examples, the CD8+ T cells are cultured in the presence of the small molecule PARP inhibitor for about 6 hours, about 12 hours, about 18 hours, about 24 hours, about 30 hours, about 36 hours, about 42 hours, about 48 hours, about 54 hours, about 60 hours, about 66 hours or about 72 hours.

In some aspects of the method, the therapeutically effective amount of the super central memory CD8+ T cells that express CD62L and CD44, CD45RO and CD62L, or CD45RO and CCR7 is about 1 to about 10 million T cells/kg, such as about 2 to about 9 million T cells/kg, about 3 to about 8 million T cells/kg, about 4 to about 7 million T cells/kg, or about 5 to about 6 million T cells/kg. In some examples, the therapeutically effective amount of the super central memory CD8+ T cells that express CD62L and CD44, CD45RO and CD62L, or CD45RO and CCR7 is about 1 million T cells/kg, about 1.5 million T cells/kg, about 2 million T cells/kg, about 2.5 million T cells/kg, about 3 million T cells/kg, about 3.5 million T cells/kg, about 4 million T cells/kg, about 4.5 million T cells/kg, about 5 million T cells/kg, about 5.5 million T cells/kg, about 6 million T cells/kg, about 6.5 million T cells/kg, about 7 million T cells/kg, about 7.5 million T cells/kg, about 8 million T cells/kg, about 8.5 million T cells/kg, about 9 million T cells/kg, about 9.5 million T cells/kg, or about 10 million T cells/kg.

The subject can be treated with any type of immunotherapy, and treatment can be administered either before administration of the super central memory CD8+ T cells, concurrent with administration of the super central memory CD8+ T cells, or following administration of the super central memory CD8+ T cells (or any combination thereof).

In some examples, the immunotherapy includes administration of a monoclonal antibody, such as a monoclonal antibody that specifically targets a tumor antigen. In some examples, the monoclonal antibody is an anti-VEGF (e.g., bevacizumab), anti-FGFR4, anti-mesothelin, anti-Her2/neu, anti-CD38, anti-CCR4, anti-CD19, anti-CD20, anti-VEGFR2, anti-CD30, anti-CD33, or anti-CD22 antibody (see, e.g., Zahavi and Weiner, Antibodies (Basel) 9 (3): 34, 2020, for examples of monoclonal antibodies that can be used for cancer immunotherapy).

In other examples, the immunotherapy includes immune cells (such as T cells, B cells, macrophages, dendritic cells or induced pluripotent stem cells) expressing tumor-targeted chimeric antigen receptors (CARs). For example, the CARs can include a monoclonal antibody or antigen-binding fragment of a monoclonal antibody that specifically binds a tumor antigen. CAR T cell (or CAR immune cell) targets can include, for example, CD123, CLL-1, CD19, CD7, CD33, CD38, CD70, FLT3, B7-H3 (CD276), CD64, TIM-3, and CD13 (see, e.g., Schorr and Perna, Front Immunol 13:1085978, 2022).

In other examples, the immunotherapy includes administration of an immune checkpoint inhibitor. In specific examples, the checkpoint inhibitor is an agent that inhibits CTLA-4 (e.g., ipilimumab), PD-1 (e.g., pembrolizumab or nivolumab), PD-L1 (e.g., atezolizumab, avelumab or durvalumab), or a costimulatory agonist antibody, such as an OX40 or GITR antibody (see, e.g., Shiravand et al., Curr Oncol 29 (5): 3044-3060, 2022).

In other examples, the immunotherapy includes administration of an oncolytic virus. In particular examples, the oncolytic virus is talimogene laherparepvec (T-VEC; LMLYGIC®), for the treatment of metastatic melanoma, or Teserpaturev, for the treatment of malignant glioma.

In other examples, the immunotherapy includes administration of a cancer vaccine. The cancer vaccine can be any one of a number of different types, such as a protein/peptide vaccine, a DNA or RNA vaccine, a whole cell vaccine, a dendritic cell vaccine, or a virus vaccine (see, e.g., Maeng and Berzofsky, F1000Res 2019 May 13; 8: F1000 Faculty Rev-654; Terbuch and Lopez, Vaccines (Basel) 6 (3): 52, 2018; and Lopes et al., J Exp Clin Cancer Res 38 (1): 146, 2019). In some examples, the cancer vaccine is Sipuleucel-T (PROVENGE®), Nadofaragene firadonevec (ADSTILADRIN®), or T-VEC (LMLYGIC®).

In other examples, the immunotherapy includes administration a cytokine. In some examples, the cytokine is an interferon (IFN), such as IFN-«. In some examples, the cytokine is interleukin-2 (IL-2).

The methods disclosed herein can be used to treat any type of cancer, such as any solid tumor. Exemplary cancers include, but are not limited to, breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck cancers, and lymphatic tumors (including B-cell and T-cell malignant lymphoma). In some examples, the cancer is a melanoma or a cervical cancer.

### V. Summary of Aspects

Aspect 1. An ex vivo method of producing super central memory CD8+ T cells that express CD62L and CD44, CD45RO and CD62L, or CD45RO and CCR7, comprising culturing CD8+ T cells in the presence of a poly(ADP-ribose) polymerase (PARP) inhibitor (PARPi), thereby generating super central memory CD8+ T cells that express CD62L and CD44, CD45RO and CD62L, or CD45RO and CCR7.

Aspect 2. The method of aspect 1, wherein the CD8+ T cells are peripheral blood CD8+ T cells, tumor-infiltrating CD8+ T cells, or engineered CD8+ T cells.

Aspect 3. The method of aspect 1 or aspect 2, wherein the super central memory CD8+ T cells CD62L^{int}CD44^{int}, CD45RO^{int} and CD62L^{int}, or CD45RO^{int} and CCR7^{int}

Aspect 4. The method of aspect 1 or aspect 2, wherein the super central memory CD8+ T cells express CD62L^{hi}CD44^{hi}, CD45RO^{hi} and CD62L^{hi}, or CD45RO^{hi} and CCR7^{hi}.

Aspect 5. The method of any one of aspects 1-4, wherein the PARPi is a small molecule inhibitor.

Aspect 6. The method of aspect 5, wherein the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide.

Aspect 7. The method of aspect 5 or aspect 6, wherein the CD8+ T cells are cultured in the presence of about 1 nM to about 15 μM of the small molecule inhibitor.

Aspect 8. The method of any one of aspects 5-7, wherein the CD8+ T cells are cultured in the presence of about 5 μM to about 10 μM of the small molecule inhibitor.

Aspect 9. The method of any one of aspects 5-8, wherein the CD8+ T cells are cultured in the presence of the small molecule inhibitor for at least 12 hours, at least 24 hours, at least 36 hours, at least 48 hours, about 60 hours, or at least 72 hours.

Aspect 10. The method of any one of aspects 1-9, wherein the CD8+ T cells are obtained from a subject with cancer.

Aspect 11. The method of any one of aspects 1-9, wherein the CD8+ T cells are obtained from a healthy donor.

Aspect 12. A method of treating a cancer in a subject, comprising:
culturing CD8+ T cells ex vivo with a PARP inhibitor (PARPi), thereby producing super central memory CD8+ T cells that express CD62L and CD44, CD45RO and CD62L, or CD45RO and CCR7; and
adoptively transferring the super central memory CD8+ T cells to the subject,
thereby treating the cancer in the subject.

Aspect 13. The method of aspect 12, wherein the CD8+ T cells are autologous CD8+ T cells

Aspect 14. The method of aspect 12, wherein the CD8+ T cells are allogeneic CD8+ T cells.

Aspect 15. The method of any one of aspects 12-14, wherein the CD8+ T cells are peripheral blood CD8+ T cells, tumor-infiltrating CD8+ T cells, or engineered CD8+ T cells.

Aspect 16. The method of any one of aspects 12-15, wherein the super central memory CD8+ T cells express CD62L^{int}CD44^{int}, CD45RO^{int} and CD62L^{int}, or CD45RO^{int} and CCR7^{int}.

Aspect 17. The method of any one of aspects 12-15, wherein the super central memory CD8+ T cells express CD62L^{hi}CD44^{hi}, CD45RO^{hi} and CD62L^{hi}, or CD45RO^{hi} and CCR7^{hi}.

Aspect 18. The method of any one of aspects 12-17, wherein the PARPi is a small molecule inhibitor.

Aspect 19. The method of aspect 18, wherein the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide.

Aspect 20. The method of aspect 18 or aspect 19, wherein the CD8+ T cells are cultured in the presence of about 1 nM to about 15 μM of the small molecule inhibitor.

Aspect 21. The method of any one of aspects 18-20, wherein the CD8+ T cells are cultured in the presence of about 5 μM to about 10 μM of the small molecule inhibitor.

Aspect 22. The method of any one of aspects 18-21, wherein the CD8+ T cells are cultured in the presence of the small molecule inhibitor for at least 12 hours, at least 24 hours, at least 36 hours, at least 48 hours, about 60 hours, or at least 72 hours.

Aspect 23. The method of any one of aspects 12-22, wherein the subject is further administered an immunotherapy for treating the cancer.

Aspect 24. The method of aspect 23, wherein the immunotherapy comprises:
a monoclonal antibody that specifically binds to a tumor antigen;
T cells expressing a tumor antigen-specific chimeric antigen receptor (CAR);
a checkpoint inhibitor;
an oncolytic virus;
a cancer vaccine; or
one or more cytokines.

Aspect 25. The method of any one of aspects 12-24, wherein the cancer is a solid tumor.

Aspect 26. The method of aspect 25, wherein the solid tumor is melanoma or cervical cancer.

Aspect 27. A method of enhancing an immune response to a vaccine in a subject, comprising:
administering to the subject the vaccine and a therapeutically effective amount of a PARP inhibitor (PARPi); or
administering to the subject the vaccine and a therapeutically effective amount of super central memory CD8+ T cells that express CD62L and CD44, CD45RO and CD62L, or CD45RO and CCR7.

Aspect 28. The method of aspect 27, wherein the PARPi is a small molecule inhibitor.

Aspect 29. The method of aspect 28, wherein the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide.

Aspect 30. The method of aspect 27 or aspect 28, wherein the therapeutically effective amount of the PARPi is about 2.5 mg/kg to about 5.0 mg/kg.

Aspect 31. The method of aspect 27, further comprising producing the super central memory CD8+ T cells that express CD62L and CD44, CD45RO and CD62L, or CD45RO and CCR7 by culturing CD8+ T cells with a PARPi.

Aspect 32. The method of aspect 31, wherein the CD8+ T cells are autologous CD8+ T cells.

Aspect 33. The method of aspect 31, wherein the CD8+ T cells are allogeneic CD8+ T cells.

Aspect 34. The method of any one of aspects 31-33, wherein the CD8+ T cells are peripheral blood CD8+ T cells, tumor-infiltrating CD8+ T cells, or engineered CD8+ T cells.

Aspect 35. The method of any one of aspects 27 and 31-34, wherein the super central memory CD8+ T cells express CD62L^{int}CD44^{int}, CD45RO^{int} and CD62L^{int}, or CD45RO^{int} and CCR7^{int}.

Aspect 36. The method of any one of aspects 27 and 31-34, wherein the super central memory CD8+ T cells express CD62L^{hi}CD44^{hi}, CD45RO^{hi} and CD62L^{hi}, or CD45RO^{hi} and CCR7^{hi}.

Aspect 37. The method of any one of aspects 31-36, wherein the PARPi is a small molecule inhibitor.

Aspect 38. The method of aspect 37, wherein the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide.

Aspect 39. The method of aspect 37 or aspect 38, wherein the CD8+ T cells are cultured in the presence of about 1 nM to about 15 UM of the small molecule inhibitor.

Aspect 40. The method of any one of aspects 37-39, wherein the CD8+ T cells are cultured in the presence of about 5 μM to about 10 μM of the small molecule inhibitor.

Aspect 41. The method of any one of aspects 37-40, wherein the CD8+ T cells are cultured in the presence of the small molecule inhibitor for at least 12 hours, at least 24 hours, at least 36 hours, at least 48 hours, about 60 hours, or at least 72 hours.

Aspect 42. The method of any one of aspects 27 and 31-41, wherein the therapeutically effective amount of the super central memory CD8+ T cells expressing CD62L and CD44, CD45RO and CD62L, or CD45RO and CCR7 is about 1 to about 10 million T cells/kg.

Aspect 43. The method of any one of aspects 27-42, wherein the vaccine is a cancer vaccine.

Aspect 44. The method of any one of aspects 27-42, wherein the vaccine is a viral or bacterial vaccine.

Aspect 45. A method of treating a cancer in a subject, comprising:
administering to the subject an immunotherapy and a therapeutically effective amount of a PARP inhibitor (PARPi), thereby treating the cancer in the subject; or
administering to the subject an immunotherapy and a therapeutically effective amount of super central memory CD8+ T cells that express CD62L and CD44, CD45RO and CD62L, or CD45RO and CCR7.

Aspect 46. The method of aspect 45, wherein the PARPi is a small molecule inhibitor.

Aspect 47. The method of aspect 46, wherein the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide.

Aspect 48. The method of aspect 46 or aspect 47, wherein the therapeutically effective amount of the PARPi is about 2.5 mg/kg to about 5.0 mg/kg.

Aspect 49. The method of aspect 45, further comprising producing the super central memory CD8+ T cells that express CD62L and CD44, CD45RO and CD62L, or CD45RO and CCR7 by culturing CD8+ T cells with a PARPi.

Aspect 50. The method of aspect 49, wherein the CD8+ T cells are autologous CD8+ T cells.

Aspect 51. The method of aspect 49, wherein the CD8+ T cells are allogeneic CD8+ T cells.

Aspect 52. The method of any one of aspects 49-51, wherein the CD8+ T cells are peripheral blood CD8+ T cells, tumor-infiltrating CD8+ T cells, or engineered CD8+ T cells.

Aspect 53. The method of any one of aspects 45 and 49-52, wherein the super central memory CD8+ T cells express CD62L^{int}CD44^{int}, CD45RO^{int} and CD62L^{int}, or CD45RO^{int} and CCR7^{int}

Aspect 54. The method of any one of aspects 45 and 49-52, wherein the super central memory CD8+ T cells express CD62L^{hi}CD44^{hi}, CD45RO^{hi} and CD62L^{hi}, or CD45RO^{hi} and CCR7^{hi}

Aspect 55. The method of any one of aspects 49-54, wherein the PARPi is a small molecule inhibitor.

Aspect 56. The method of aspect 55, wherein the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide.

Aspect 57. The method of aspect 55 or aspect 56, wherein the CD8+ T cells are cultured in the presence of about 1 nM to about 15 μM of the small molecule inhibitor.

Aspect 58. The method of any one of aspects 55-57, wherein the CD8+ T cells are cultured in the presence of about 5 μM to about 10 μM of the small molecule inhibitor.

Aspect 59. The method of any one of aspects 55-58, wherein the CD8+ T cells are cultured in the presence of the small molecule inhibitor for at least 12 hours, at least 24 hours, at least 36 hours, at least 48 hours, about 60 hours, or at least 72 hours.

Aspect 60. The method of any one of aspects 45 and 49-59, wherein the therapeutically effective amount of the super central memory CD8+ T cells that express CD62L and CD44, CD45RO and CD62L, or CD45RO and CCR7 is about 1 to about 10 million T cells/kg.

Aspect 61. The method of any one of aspects 45-60, wherein the immunotherapy comprises:
a monoclonal antibody that specifically binds to a tumor antigen;
immune cells (such as T cells) expressing a tumor antigen-specific chimeric antigen receptor (CAR);
a checkpoint inhibitor;
a co-stimulatory agonist antibody;
an oncolytic virus;
a cancer vaccine; or
one or more cytokines.

Aspect 62. The method of any one of aspects 45-61, wherein the cancer is a solid tumor.

Aspect 63. The method of aspect 62, wherein the solid tumor is melanoma or cervical cancer.

### EXAMPLES

The following examples are provided to illustrate particular features of certain aspects of the disclosure, but the scope of the claims should not be limited to those features exemplified.

The Examples below report the direct effect of PARPi on CD8 T cells. It is demonstrated that inhibition of PARP results in CD8 T cell reprograming, leading to generation of a unique T_{CM} population (T_{CM}^{sup}) that expresses a higher level of memory markers, and shows greater memory recall responses and anti-tumor effects. It was found that PARPi induces this unique population in both mouse and human CD8 T cells. Notably, an increase in the percentage of T_{CM} with higher CD62L expression was found in CD8 T cells obtained from patients with ovarian cancer that were treated with olaparib. Similar to the findings in ovarian cancer patients, an increase in the memory CD8 T cell population has been reported in metastatic colorectal cancer patients treated with the combination of temozolomide and olaparib (Cecchini et al., Cancer Res Commun 3 (6): 1132-1139, 2023), suggesting a role of PARPi in memory CD8 T cell differentiation.

It is well established that metabolic reprogramming is one of the key mechanisms that modulates T cell activation and differentiation (Zhang and Romero, Trends Mol Med 24 (1): 30-48, 2018) and that memory cells rely more on OXPHOS and FAO than glycolysis for their function (Zhang and Romero, Trends Mol Med 24 (1): 30-48, 2018). Indeed, it is disclosed herein that PARPi reduces glycolysis and enhances FAO and OXPHOS that are associated with memory CD8 T cells. This results in metabolic reprograming that supports the generation of highly metabolically fit T_{CM}^{sup} cells with stronger immune-mediated anti-tumor activity. Similar to these effects of PARPi on metabolic reprograming in CD8 T cells, it has also been recently reported that PARP inhibitors can directly modulate the metabolism of tumor associated macrophages by decreasing glycolysis and shifting toward pathways associated with lipid metabolism (Mehta et al., Nat Cancer 2 (1): 66-82, 2021). Interestingly, impaired glucose uptake in CD8 T cells was also reported in tumor-bearing mice after olaparib treatment (Mehta et al., Nat Cancer 2 (1): 66-82, 2021). It is known that PGC1-α inhibits glycolysis and enhances FAO in CD8 T cells (Cruz-Bermudez et al., Free Radic Biol Med 135:167-181, 2019). Since the studies described below found that PARPi increases the level of PGC1-α in CD8 T cells, it is believed that this induction may represent one of the mechanisms for this metabolic reprograming and the generation of T_{CM}^{sup}. Another important mechanism for this metabolic switch mediated by PARPi could be its ability to regulate the expression of metabolic genes at the transcriptional level (Saenz et al., BMC Genomics 9:171, 2008; Kraus and Lis, Cell 113 (6): 677-683, 2003). Indeed, a significant decrease in glycolysis related genes and an increase in FAO related genes in T_{CM}^{sup} was found. Moreover, in addition to their enhanced metabolic fitness, T_{CM}^{sup} showed a higher cytotoxicity compared to T_{CM} cells as determined by high expression level of both INF-γ and Gzm B. These transcriptional changes could be explained by the PARPi-mediated increased trapping of PARP in the nucleus as reported previously (Murai et al., Cancer Res 72 (21): 5588-5599, 2012). In fact, PARPi-treated cells have an increase in PARP in the nucleus independent of its enzymatic activity. This is further strengthened by the lack of generation of T_{CM}^{sup} when PARP siRNA was used that unlike PARPi does not induce PARP trapping. Interestingly, PARPi-mediated increased trapping of PARP is also known to exclude H1 from promoters of genes related to T cell activation and effector function, repressing their transcription (Krishnakumar et al., Science 319 (5864): 819-821, 2008; Willcockson et al., Nature 589 (7841): 293-298, 2021), which may provide another potential explanation. These findings indicate the crucial role of PARPi-mediated PARP trapping in generation of T_{CM}^{sup} cells by inducing a transcriptional switch.

Our in vitro, in vivo, and clinical findings showed that inhibiting PARP can increase the expression of memory markers, including CD62L, CCR7 and Eomes. These genes are regulated by FOXO1 a transcription factor known to be a main driver for CD8 T cell memory generation (Ouyang et al., Immunity 30 (3): 358-371, 2009), for which one of the mechanisms could be a reduced methylation at certain pro-memory genes mediated by FOXO1 (Gray et al., Immunity 46 (4): 596-608, 2017). FOXO1 expression is also known to be regulated by SIRT1, a protein deacetylase (Chung and Joe, Integr Med Res 3 (4): 198-203, 2014). In addition, activation of the SIRT-1/FOXO1 axis can prevent terminal differentiation of T cells and maintain their memory phenotype (Jeng et al., J Exp Med, 215 (1): 51-62, 2018). Indeed, it is shown herein that inhibiting PARP enhances SIRT1/FOXO1 pathway activation, leading to T_{CM}^{sup} generation that was confirmed by using both SIRT1 and FOXO1 inhibitors.

The findings from mouse tumor models after treatment with either PARPi or with ACT of PARPi-treated CD8 T cells demonstrate that the direct effects of PARPi on CD8 T cells, resulting in their reprogramming into T_{CM}^{sup} is important for the enhanced anti-tumor immune response. These findings also provide a plausible explanation to previously reported data that demonstrate re-challenge of mice showing complete response to olaparib treatment resulted in complete tumor rejection (Staniszewska et al., Oncoimmunology 11 (1): 2083755, 2022). Similarly, it also further elucidates the reason for the improved anti-tumor efficacy of CAR-T cells treated in vitro with PARPi in mice bearing breast tumors, where the CAR-T indeed showed an increase in the percentage of CD44⁺CD62L⁺ population and INFγ (Sun et al., Mol Ther 29 (1): 60-74, 2021).

Based on the above, a model explaining the direct effect of PARPi on CD8 T cells is proposed, leading to generation of T_{CM}^{sup} that show enhanced recall responses (FIG. 10). It is shown herein that both the inhibition of the enzymatic activity of PARP and the trapping of PARP in the nucleus contribute to metabolic and transcriptional switches, leading to the development of T_{CM}^{sup}. Briefly, the inhibition of the enzymatic activity of PARP results in NAD accumulation and, in turn, an increase in SIRT-1, which mediates an increase in PGC1-α expression. This results in an increase in FAO (Cruz-Bermudez et al., Free Radic Biol Med 135:167-181, 2019). Furthermore, PARPi-induced PARP trapping mediates transcriptional modification that induces the expression of metabolism related genes, leading to inhibition of glycolysis and an increase of FAO. In addition, increased SIRT1 activity results in FOXO1 deacetylation, which leads to an increase in FOXO1 protein accumulation in the nucleus, and hence, the expression of FOXO1 target genes related to the memory phenotype.

Compared to terminally differentiated cells, growing evidence supports the importance of less differentiated memory T cells in generating strong anti-tumor immune responses. This is also shown to be correlated with improved response shown by cancer patients. In light of these reports, generation of T_{CM}^{sup} after PARP inhibition that have significantly higher memory potential including antigen-specific recall responses and more potent anti-tumor functionality compared to T_{CM} cells will support development of various combination strategies and will enhance the efficacy of cellular therapies such as ACT/CAR-T.

### Example 1: Materials and Methods

This example describes the materials and experimental procedure for the studies described in Examples 2-6.

### Mice and Cell Culture

Four-to-six-week-old C57BL/6J mice (wild type) were purchased from Jackson Laboratory. In-house-bred pMel-1 mice [B6.Cg-Thy1a/Cy Tg (TcraTcrb) 8Rest/J], which carry a rearranged TCR transgene (Vβ13) specific for the mouse homolog (pmel-17) of human gp100 (Ji et al., PLoS One 9(5):e96650, 2014), with transgenic CD8 T cells having a melanoma-gp100 peptide-specific TCR were used as outlined in various experiments. Animals had free access to water and food. Cancer cell lines used in the present study included TC-1 (Lin et al., Cancer Res 56(1):21-26, 1996) and B16F10 melanoma (obtained from the American Type Culture Collection (ATCC)). Cell lines were routinely tested for absence of any contamination, including mycoplasma, by microscopic evaluation and PCR-based methods. Primary murine CD8 T cells, isolated by negative selection using magnetic beads (Miltenyi Biotec), were cultured in RPMI 1640 medium supplemented with 10% FBS, 2 mM glutamine, 10 mM HEPES and 55 μM β-mercaptoethanol. Purity of all the cell populations was greater than 95%.

### Tumor Establishment and Mice Treatment

C57BL/6J mice were injected with 7×10⁴ TC-1 cells per mouse or 1×10⁵B16F10 cells per mouse in the right flank. Treatment in respective groups was started when tumors reached an average size of approximately 0.075 cm³. PARP inhibitor treatment was performed using olaparib (AZ12627831-036) obtained from AstraZeneca. Mice were treated daily with IP injection at a dose of 50 mg kg-1.

For vaccination, TC-1-specific E7 peptide (a nine amino acid peptide, RAHYNIVTF (SEQ ID NO: 1); 100 μg per mouse per 100 μl; Celtak Bioscience) or B16-specific gp10025-33 nonamer peptide (KVPRNQDWL (SEQ ID NO: 2); 100 μg per mouse per 100 μl; AnaSpec) were used. Both the vaccines were mixed with a small 13-mer nonnatural pan-HLA DR-binding epitope (PADRE (SEQ ID NO: 3); aK-Cha-VAAWTLKAAa (SEQ ID NO: 4), where ‘a’ is d-alanine, and ‘Cha’ is 1-cyclohexylalanine, T helper epitope, 20 μg per mouse; Celtak Bioscience) and QuilA (adjuvant, 10 μg per mouse; Brenntag). For survival and therapy experiments, mice were vaccinated three times, starting at an average tumor size of 0.07 cm³, while for immune response experiments mice were vaccinated twice with a one-week interval between vaccinations. Tumors were measured every 3-4 days using a digital vernier caliper, and the tumor volume was calculated using the formula: V=L×W2/2000, where V is tumor volume, L is the length of the tumor (longer diameter) and W is the width of the tumor (shorter diameter). Mice were monitored for tumor growth and survival. Mice were euthanized when the tumor volume reached 1.5 cm³.

### Tumor Collection and Sample Preparation

Two-to-three days after the second vaccination, mice in the various groups were euthanized and tumors were collected. Samples were processed using a gentle MACS dissociator and the solid tumor homogenization protocol, as suggested by the manufacturer (Miltenyi Biotec).

### Cell Activation, Drug Treatment and Adoptive Transfer

CD8+ T cells from pMel-1 mice were activated with gp100 peptide (KVPRNQDWL (SEQ ID NO: 2); 1 μM per 10⁶ cells per ml) either alone or in combination with PARPi (5 or 10 μM) in T cell medium supplemented with 30 international units (IU) of IL-2 for 48 h unless otherwise stated. For ACT experiments, CD8+ T cells from pMel-1 mice were activated with gp100 peptide with and without PARPi for 48 h. Based on their CD44 and CD62L expression, cells were sorted by FACS and transferred into 9-day-old B16F10 melanoma-bearing mice tumor-bearing mice (1×10⁶ cells per mouse). Tumor growth and survival of mice were monitored.

For evaluation of memory characteristics in human CD8+ T cells, naive CD8+ T cells were activated in the presence or absence of PARPi, by Dynabeads™ Human T-Activator CD3/CD28 (Thermo Fisher #11131), for 48 h. Post-activation cells were gated upon CD8⁺ T cell population and various characteristics were investigated as described in the Examples below.

### Flow Cytometry Analyses

A total of 0.1-0.2×10⁶ cells per sample were stained for Live/Dead staining followed by fixation and permeabilization. For IFNγ and granzyme B (Gzm B) staining, BD Cytofix/Cytoperm (catalog no. 51-2090KZ) and BD Perm/Wash (catalog no. 51-2091KZ) buffer set were used according to the manufacturer's instructions (BD Pharmingen). CD8⁺ T cells from tumor samples were stained similarly. Data acquisition was performed on a BD LSR II flow cytometer using the software FACS Diva v.9.0. Results were analyzed using FlowJo software (TreeStar). MitoFM, a green-fluorescent mitochondrial stain, which localizes to mitochondria, was used as described previously (Sukumar et al., Cell Metab 23 (1): 63-76, 2016).

### Antibodies and Reagents

The Live/Dead Fixable Near-IR Dead Cell Stain Kit (catalog no. L34976) and CellTrace Violet (VCT) Cell Proliferation Kit (catalog no. C34557) were obtained from Thermo Fisher Scientific. BD Cytofix/Cytoperm (catalog no. 51-2090KZ) and BD Perm/Wash (catalog no. 51-2091KZ). The fluorochrome-labeled anti-mouse antibodies used for flow cytometry measurements in vitro and in vivo were: CD3 (BD #562286); CD8 (BD #557959); E7 (ImmuDEx #JA2195-FITC); KI67 (Invitrogen #56-5698-82); INFγ (BD #557724; #554412); Gzm B (Biolegend #372204); CD62L (BD #560507); CD44 (BD #553133, #560567, #559250); Eomes (Invitrogen #53-4875-82); BCl2 (Invitrogen #12-6992-42); CCR7 (BD #560805); H2AX (Invitrogen #53-9865-82). The fluorochrome-labeled anti-human antibodies used for flow cytometry measurements in vitro were: CCR7 (BD #561143); CD45RO (BD #559865); CD45RA (BD #555488); CD62L (Biolegend #304826); INFγ (BD #502522); Gzm B (Biolegend #515408), CD8 (clone SK1) Biolegend Ki-67 (clone B56) BD biosciences. Other reagents included Talazoparib (Selleckchem #BMN673) Niraparib (Selleckchem #MK-4827), Sirt1 inhibitor (Selleckchem #S1541), FOXO1 inhibitor (Millipore Sigma #AS1842856), Etomoxir (Millipore Sigma #828934-41-4), and SR-18292 Maleate (Millipore Sigma #2095432-55-4).

### Metabolic Assays for Estimation of Various Metabolic Characteristics

CD8 T cells were activated with gp100 peptide with/without PARPi for 48 h, followed by mitochondrial stress tests (SeaHorse Bioscience), performed as per the manufacturer's specifications. OCR and ECAR were measured with an XFp flux analyzer (Seahorse Bioscience). For all assays, 160,000 cell per ml were plated onto culture plates using Cell-Tak (BD Biosciences). OCR and ECAR were measured in unbuffered DMEM (Agilent Biotechnologies) supplemented with 10 mM D-glucose (Sigma-Aldrich), 10 mM L-glutamine and 2.5 mM pyruvate, as indicated. The rates of FAO were determined in variously treated cells, as described above, using a Seahorse XF Palmitate-BSA FAO Reagent kit, used according to the manufacturer's instructions (Agilent Biotechnologies).

### Western Blot

Negatively isolated murine pMel-1 CD8 T cells were activated as described above. Cell lysates (RIPA buffer+1% phosphatase inhibitor+1% protease inhibitor) of pMel-1 CD8+ T cells were prepared following various treatments. Protein concentrations in cell lysates were determined by Pierce BCA Protein Assay Kit (Thermo Fisher Scientific). Protein (30-40 μg) was loaded onto Novex 4-12% Tris-Glycin Mini Gels (Thermo Fisher Scientific) followed by transfer onto nitrocellulose membranes. Membranes were blocked with 5% BSA in Tris buffer followed by overnight probing with antibodies against p-FOX01 (Cell signaling, 9461T), Total FOXO1 (Cell signaling, 9475S), SIRT1 (Cell signaling, 2880S), Ac-FOXO1 (MBS9600633), PAR (R&D, 4335-MC-100) and PARP1 (Cell signaling, 9542s) by western blot. The blots were developed with appropriate (anti-rabbit or anti-mouse) HRP-conjugated secondary antibodies. The level of β-actin was used as a control. Densitometric analysis of the bands was performed using a software (Image Studio Lite v.5.0) from LI-COR (online at licor.com/bio/image-studio-lite/).

### SiRNA PARP

For knockdown of PARP in pMel-1 CD8+ T cells. Activated cells were incubated overnight with self-delivering AUMsilence oligos (10 μM; a mixture of 4 siRNAs) designed and synthesized by AUM BioTech (Philadelphia, PA, USA) (Zhang et al., Science 381(6656):eadd6696, 2023; Marasca et al., Nature Genetics 54(2):180-193, 2022). Cells from the appropriate treatment (scrambled (scRNA) versus siRNA) were tested for the knockdown of PARP by flow cytometry using rabbit-anti-PARP1 (Cell signaling, 9542) and PAPR2 (Proteintech, #55149-1-AP) followed by secondary rabbit antibody coupled to Alexa Fluor 488 (Invitrogen, 35552).

Quantitative PCR with Reverse Transcription Analysis

Total RNA was extracted from gp100 or gp100 plus PARPi-activated CD8 T cells using the RNeasy Mini Kit (Qiagen, catalog no. 74004) following the manufacturer's instructions. The extraction included a digestion step with DNase I to prevent subsequent amplification of genomic DNA. One μg total RNA was subjected to single-strand complementary DNA synthesis using High-Capacity cDNA Reverse Transcription Kit (Thermo Fisher, catalog no. 4368814). The Taqman® gene expression assay from Applied Biosystems was used for qrtPCR, and primer pairs (catalog no. 4331182) from Applied Biosystems were used to detect gene expression of Eomes (#Mm01351984-m1), Acot1 (#Mm0162271-S1), Cpt1a (#Mm0123183-m1), Klf2 (#Mm00500486-g1), CD62L (#Mm00441291-m1),CCR7 (#Mm99999130-s1), and Cdkn1b (#Mm00438168-m1). Data were procured using StepOnePlus Real-Time PCR System from Applied Biosystems and normalized to the geometric mean of the housekeeping gene beta-actin (β-actin) (#Mm02619580-g1).

### Immunofluorescence

pMel-1 CD8 T cells were activated with gp100 in the presence or absence of PARPi for 48 h containing glass coverslips. Cells were then washed with PBS (Mg⁺ca⁺), fixed for 5 min with 4% PFA and permeabilized in PBS/triton 1×. Coverslips were blocked and stained with primary antibody FOXO1 (Cell signaling, 9475S) overnight followed by secondary rabbit antibody coupled to Alexa Fluor 488. Finally, DAPI staining was applied. Images were acquired with a Leica TCS-SP5II upright confocal microscope (Leica Microsystems).

### NAD Accumulation Assay

pMel-1 CD8 T cells were activated with gp100 in the presence or absence of PARPi for 48 h. Cells were washed with cold PBS and pelleted in a microcentrifuge tube by centrifuging at 2,000 rpm for 5 minutes. Cells were lysed with 400 μL of NADH/NAD extraction buffer and then centrifuged at 13,000 g for 10 minutes to remove insoluble material. The supernatants were collected and the levels of NAD in the cell lysates were estimated using the NAD/NADH Quantitation Kit according to the manufacturing kit (Millipore NAD/NADH Quantification Kit Sigma #MAK037).

### Metabolomic Analysis

All LC-MS grade solvents including acetonitrile and water were purchased from Fisher Optima grade, Fisher Scientific. High purity formic acid (99%) was purchased from Thermo-Scientific. Debrisoquine and 4-nitrobenzoic acid were purchased from Sigma-Aldrich. EquiSPLASH® LIPIDOMIX® quantitative mass spec internal standard and 15:0-18:1-d7-PA, C15 Ceramide-d7 (d18:1-d7/15:0) and 18:1 Chol (D7) ester were purchased from Avanti polar lipids. Internal standard for free fatty acid (FFA), dihydroceramides (DCER), hexosylceramides (HCER), lactosylceramides (LCER) were purchased from Sciex as Lipidyzer platform kit.

### Cell Metabolomics Using QTRAP 5500

Targeted metabolomics method, developed in-house, was used to quantitate 270 endogenous metabolites using QTRAP® 7500 LC-MS/MS System (Sciex, MA, USA). For this purpose, 20 μL of PBS was added to the cell pellet and sample tube was plunged into dry ice for 30 seconds and 37° C. water bath for 90 seconds. This cycle was repeated for two more times and then samples were sonicated for 1 minute and 100 μL of extraction buffer (methanol/water 50/50) containing 200 ng/ml of debrisoquine (DBQ) as internal standard for positive mode and 200 ng/mL of 4-nitrobenzoic acid as internal standard for negative mode was added. The samples were vortexed for 1 min and kept on ice for 20 minutes followed by addition of 100 μL of ACN. The samples were incubated at −20° C. for 20 minutes for protein precipitation. The samples were centrifuged at 13,000 rpm for 20 minutes at 4° C. The supernatant was transferred to MS vial for LC-MS analysis. 20 μL of each prepared sample was mixed to generate the pooled QC sample.

NIST Plasma sample preparation: 25 μL of NIST plasma sample was dissolved in 100 μL of extraction buffer (methanol/water 50/50) containing 200 ng/ml of debrisoquine (DBQ) as internal standard for positive mode and 200 ng/mL of 4-nitrobenzoic acid as internal standard for negative mode. The sample was vortexed for 30 seconds and incubated on ice for 20 min followed by addition of 100 μL of acetonitrile and incubation at −20° C. for 20 min. Samples were centrifuged at 13,000 rpm for 20 min at 4° C. The supernatant was transferred to MS vial for LC-MS analysis.

Five microliter of the prepared sample was injected onto a Kinetex 2.6 μm polar C18 100 Å 100×3.0 mm (Phenomenex, CA, USA) using SIL-30AC autosampler (Shimadzu) connected with a high flow LC-30AD solvent delivery unit (Shimadzu) and CBM-20A communication bus module (Shimadzu) online with QTRAP 5500 (Sciex, MA, USA) operating in positive and negative ion mode. A binary solvent comprising of water with 0.1% formic acid (solvent A) and acetonitrile with 0.1% formic acid (solvent B) was used. The extracted metabolites were resolved at 0.2 mL/min flow rate. The LC gradient conditions were as follows: Initial—100% A, 0% B for 2.1 minutes; 14 minutes—5% A, 95% B till 15 minutes; 15.1 minutes—100% A, 0% B till 20 minutes. The auto sampler and oven were kept at 15° C. and 30° C., respectively. Source and gas setting for the method were as follow: curtain gas=40, CAD gas=9, ion spray voltage=1700 V in positive mode and ion spray voltage=1600 V in negative mode, temperature=350° C., ion source gas 1=30 and ion source gas 2=50.

### Lipidomics Using QTRAP 5500

This method is designed to measure 21 classes of lipid molecules which includes diacylglycerols (DAG), cholesterol esters (CE), sphingomyelins (SM), phosphatidylcholine (PC), triacylglycerols (TAG), free fatty acids (FFA), ceramides (CE), dihydroceramides (DCER), hexosylceramide (HCER), lactosylceramide (LCER), phosphatidylethanolamine (PE), lysophosphatidylcholine (LPC), lysophosphatidylethnolamine (LPE), phosphatidic acid (PA), lysophosphatidic acid (LPA), phosphatidylinositol (PI), lysophosphatidylinositol (LPI), phosphatidylglycerol (PG), acylcarnitines and phosphatidylserine (PS) using QTRAP® 5500 LC-MS/MS System (Sciex). For this purpose, 20 μL of PBS was added to cell samples and sample tube was plunged into dry ice for 30 seconds and 37° C. water bath for 90 seconds. Freeze thaw cycle was repeated for three times and then samples were sonicated for 30 seconds. To the above solution was added 100 μL of chilled isopropanol containing internal standards for lipids and samples were vortexed. The samples were vortexed again for 1 min and kept on ice for 30 minutes. Samples were incubated at −20° C. for 2 hours for complete protein precipitation. The samples were centrifuged at 13,000 rpm for 20 minutes at 4° C. The supernatant was transferred to MS vial for LC-MS analysis.

NIST Plasma sample preparation: 25 μL of NIST plasma sample was dissolved in 125 μL of chilled isopropanol containing internal standards for lipid classes. The sample was vortexed for 30 seconds and incubated on ice for 30 min followed by incubation at −20° C. for 2 hours. Samples were centrifuged at 13,000 rpm for 20 min at 4° C. The supernatant was transferred to MS vial for LC-MS analysis.

Five μL of each sample was injected onto a Xbridge amide 3.5 μm, 4.6×100 mm (waters) using SIL-30 AC auto sampler (Shimadzu) connected with a high flow LC-30AD solvent delivery unit (Shimadzu) and CBM-20A communication bus module (Shimadzu) online with QTRAP 5500 (Sciex, MA, USA) operating in positive and negative ion mode. A binary solvent comprising of acetonitrile/water 95/5 with 10 mM ammonium acetate as solvent A and acetonitrile/water 50/50 with 10 mM ammonium acetate as solvent B was used for the resolution. Lipids were resolved at 0.7 mL/min flow rate, initial gradient conditions started with 100% of solvent A, shifting towards 99.9% of solvent A over a time period of 3 minutes, 94% of solvent A over a time period of 3 minutes and 25% of solvent A over a period of 4 minutes. Finally, washing with 100% of B for 6 minutes and equilibrating to initial conditions (100% of solvent A) over a time period of 6 minutes using auto sampler temperature 15° C. and oven temperature 35° C. Source and gas setting were as follow: curtain gas=30, CAD gas=medium, ion spray voltage=5.5 kV in positive mode and −4.5 kV in negative mode, temperature=550° C., nebulizing gas=50 and heater gas=60.

### Data Processing

The data were normalized to respective internal standard area and processed using MultiQuant 3.0.3 (Sciex). The quality and reproducibility of LC-MS data was ensured using a number of measures. The column was conditioned using the pooled QC samples initially and were also injected periodically to monitor shifts in signal intensities and retention time as measures of reproducibility and data quality of the LC-MS data. NIST plasma sample was also run, periodically, prepared in the same manner to check the instrumental variance. Blank solvent runs were used between set of samples to minimize carry-over effects.

### RNA-Sequencing

Ribo-Zero Plus Kit from Illumina® was used for stranded Total RNA Prep Ligation. This protocol involves the removal of ribosomal RNA (rRNA) using biotinylated, target-specific oligos combined with Ribo-Zero rRNA removal beads. RNA was fragmented into small pieces and the cleaved RNA fragments were copied into first strand cDNA using reverse transcriptase and random primers, followed by second strand cDNA synthesis using DNA Polymerase I and RNase H. The resulting double-strand cDNA were used as the input to a standard Illumina library prep with end-repair, adapter ligation and PCR amplification being performed to give a sequencing ready library. The final purified product was then quantitated by qPCR before cluster generation and sequencing on NovaSeq 6000 instrument.

### Patient Samples

Paired (pretreatment and on treatment cycle 2 day 1) PBMC samples were obtained from 11 patients with recurrent ovarian cancer enrolled in a phase 2 trial of olaparib and durvalumab (NCT02484404) (Lampert, et al., Clin Cancer Res 26 (16): 4268-4279, 2020). Peripheral blood mononuclear cells (PBMCs) were isolated from whole blood collected in cell preparation tubes (CPT) with sodium citrate and viably frozen until analysis. After thawing, PBMCs were washed with flow buffer (5% BSA, 2 mM EDTA in PBS), incubated with Fc receptor blocking reagent (Miltenyi Biotec) and stained with monoclonal antibodies for 20-30 min at 4° C. For staining for Ki-67 and Granzyme B, cells were fixed and permeabilized using a Fix/Perm buffer (eBiosciences) according to the manufacturer's instructions. Dead cells were excluded from the analysis by using the viability dye, LIVE/DEAD Fixable (Life Technologies). All assays were performed using multiparametric flow cytometry (MACSQuant; Miltenyi Biotec). Data were analyzed using FlowJo software v.10.6.1 (FlowJo, LLC). Flow cytometric data were quantified either as the median fluorescence intensity or as a percentage of cells, as indicated.

### Statistical Analysis

Sample sizes were determined by prior experience and to achieve a confidence level of at least 95%. All summary statistics (average values, s.d., s.e.m., significant differences between groups) were calculated using GraphPad Prism v.6.0 or Excel, as appropriate. Statistical significance between groups was determined by unpaired, one-tailed Student's t-test or one-way analysis of variance (ANOVA) (P≤0.05 was considered statistically significant). Survival in various groups was compared with GraphPad Prism using log-rank (Mantel-Cox) test. SK plots were generated by internally developed software (online at skylineplotter.shinyapps.io/SkyLinePlotter/). Contrary to the survival plot generated using GraphPad Prism, the SK plot gives dynamic simultaneous presentation of tumor volumes and mouse survival at a specific time point.

### Example 2: PARP Inhibition Enhances Anti-Tumor Immune Response by Modulating CD8 T Cells Function and Differentiation

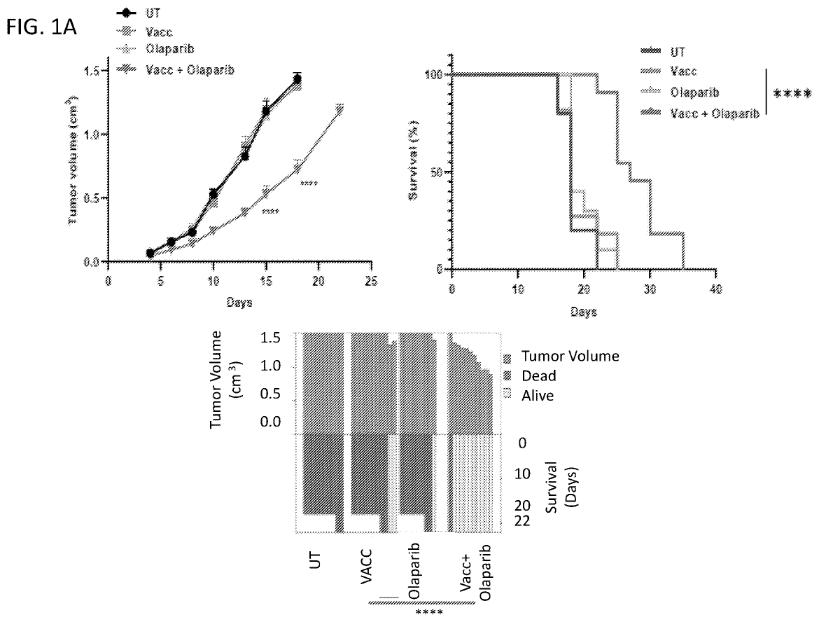

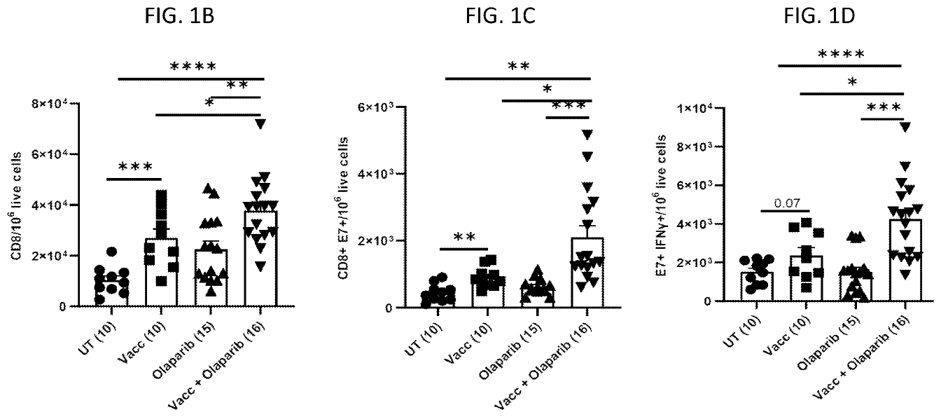

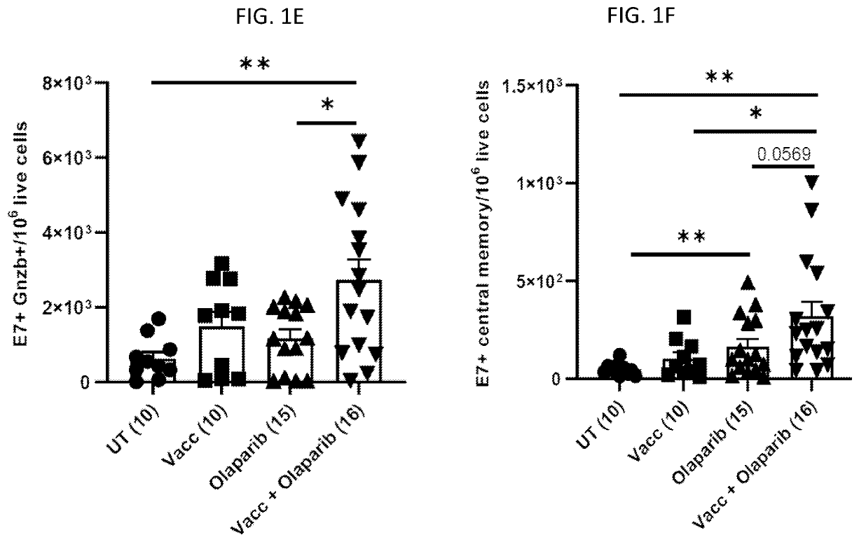

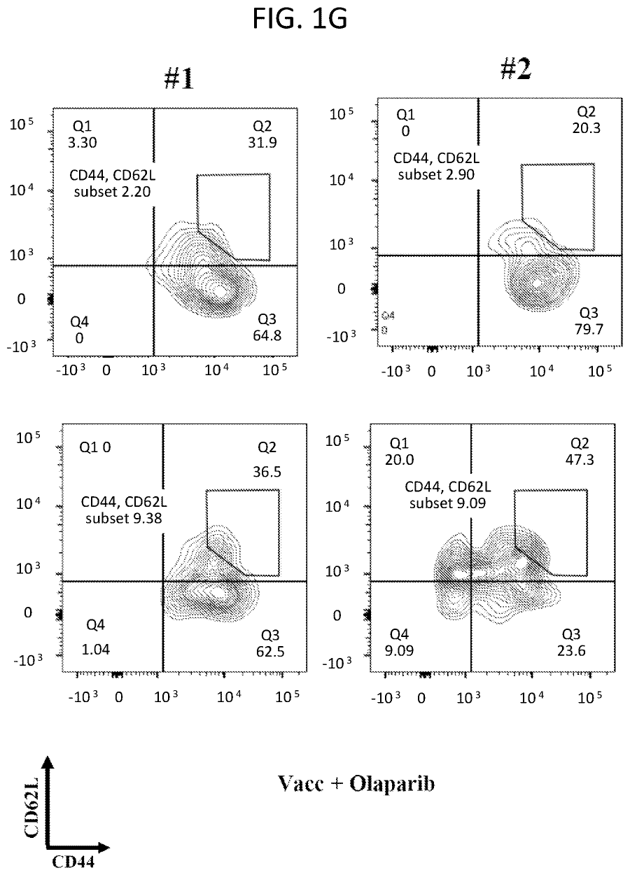

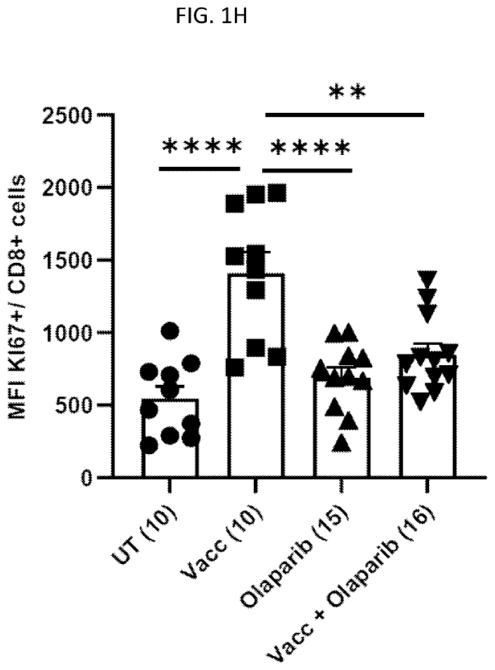

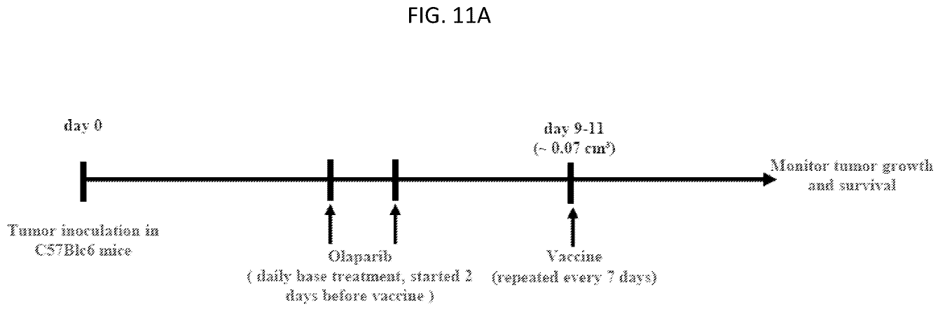

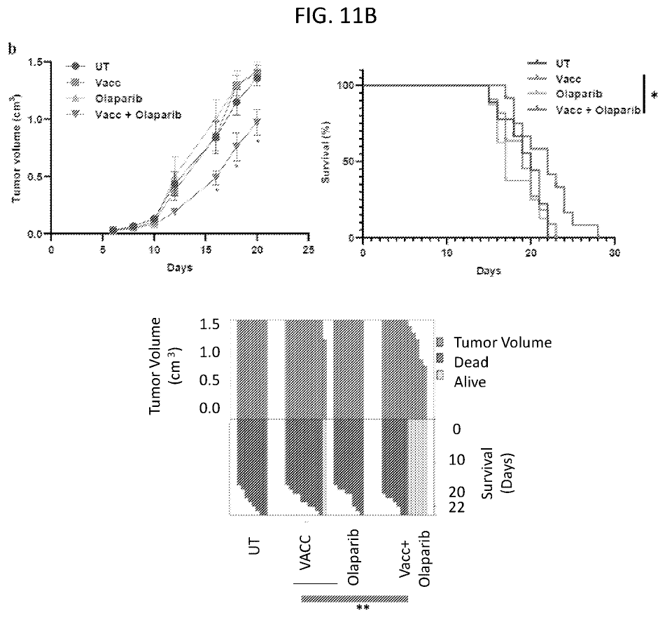

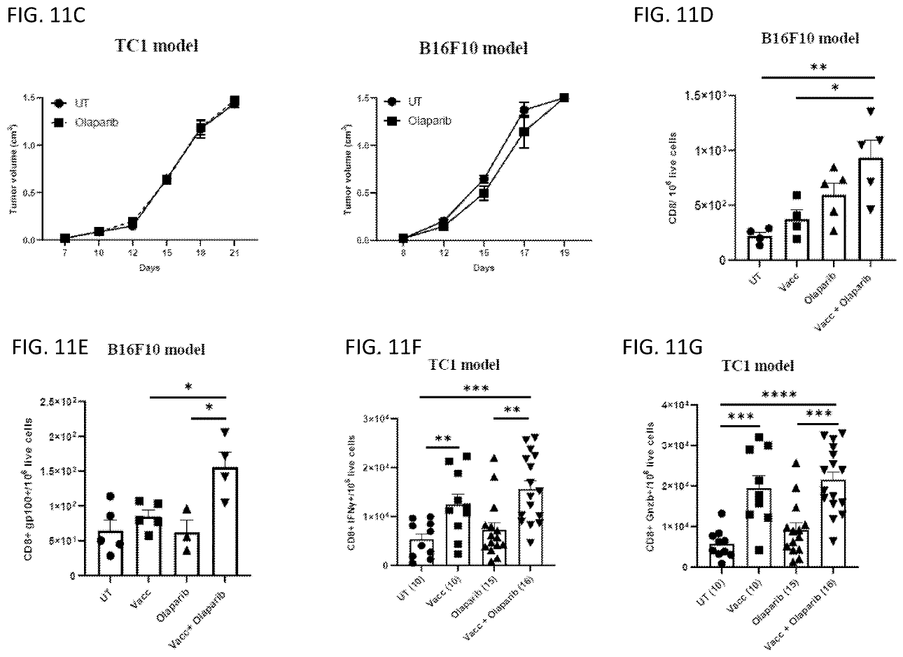

The effect of PARPi on direct CD8 T cell-driven anti-tumor immune response remains poorly understood. Therefore, to dissect this effect, the anti-tumor immune effect of PARPi was tested in vivo in tumor models that require T cell priming, which helps delineate the direct effect of the drug on CD8 T cells. Accordingly, mice bearing TC-1 or B16F10 tumors were treated with the PARPi olaparib (AZ12627831-036), in combination with the corresponding vaccine, HPV E7 or gp100, respectively. Mice were treated according to the schedule shown in FIG. 11A. It was found that PARPi significantly enhanced the anti-tumor immune response of the vaccine by significantly retarding tumor growth and prolonging survival of mice in both tumor models (FIG. 1A and FIG. 11B). While 18% and 10% of mice survived in the vaccine-treated and PARPi-treated groups, respectively, in the TC-1 tumor model at day 22, 90% survived when PARPi was added to the vaccine. Similarly, in the B16F10 tumor model, 10% of the mice treated with vaccine alone were alive at day 22, compared to 40% when PARPi was added. PARPi had no single agent effect in B16 model either (FIG. 11B). In addition, PARPi did not affect tumor growth of TC-1 and B16F10 in immune deficient nude mice (FIG. 11C). All of the above suggests that anti-tumor effect of PARPi is mediated though the enhancement of immune response.

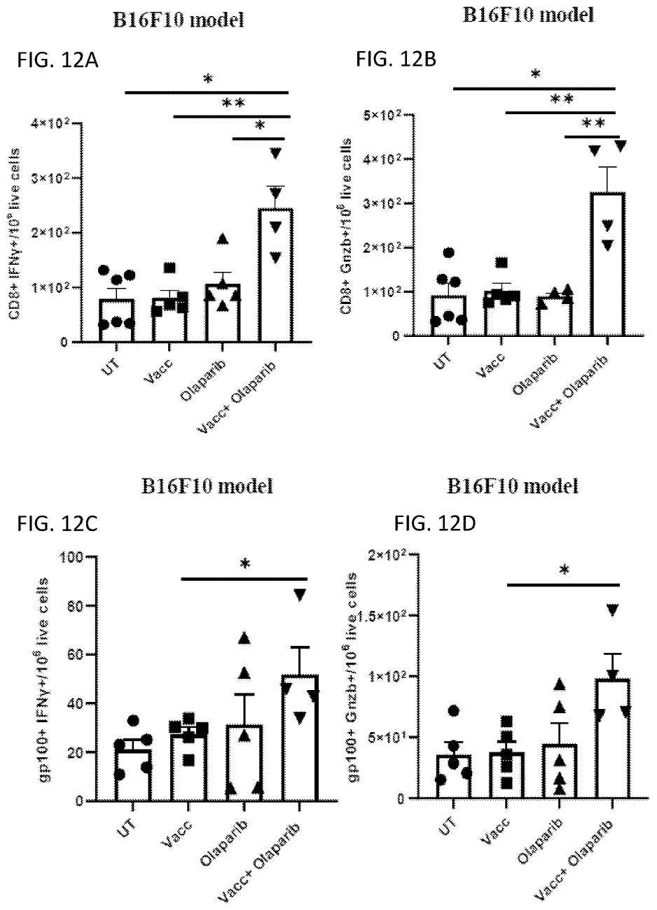

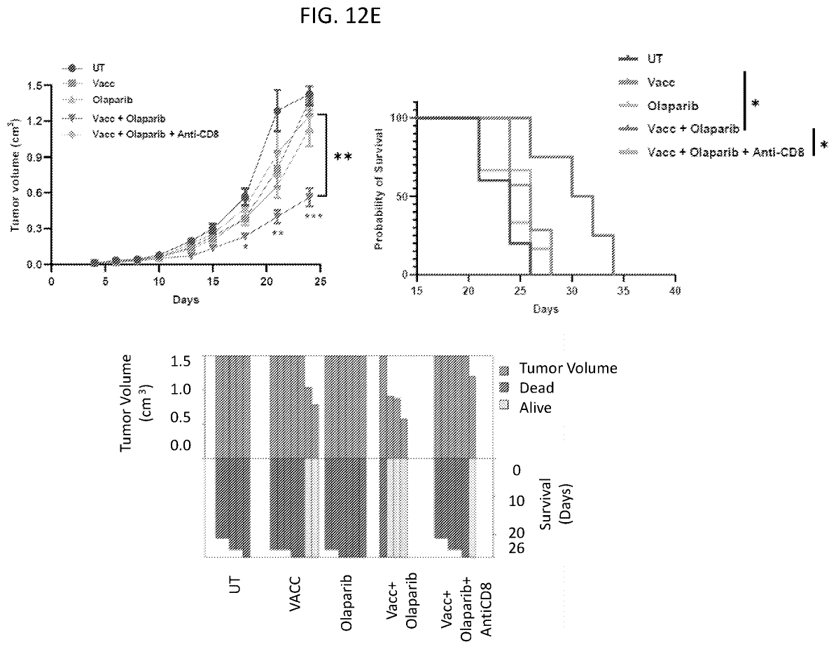

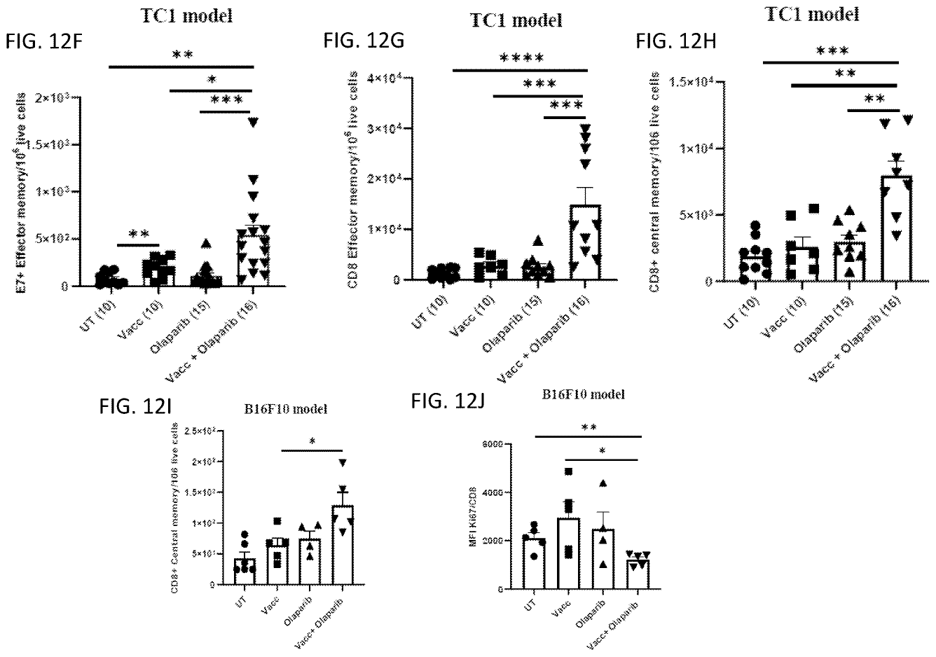

To understand the mechanism by which PARPi enhances the anti-tumor immune response, immune profiling of the TME from TC-1 and B16F10 tumor-bearing mice was conducted. The results showed a significant increase in the number of total and antigen-specific CD8⁺ T cells in TME of mice vaccinated in the presence of PARPi compared to either of the treatments alone in TC1 (FIGS. 1B-1C) and B16 (FIGS. 11D-11E). Furthermore, PARPi enhanced functionality of total and antigen-specific CD8⁺ T cells by increasing both IFNγ and granzyme B+ (Gzm B) cells when given with the vaccine (FIGS. 1D-1E, 11F-11G and 12A-12D). To further confirm the effect of PARPi on CD8 T cells, the effect of the treatment in the presence or absence of CD8 T cell depleting antibodies was tested. The results showed that depletion of CD8 T cells in TC-1 tumor model led to the loss of the anti-tumor effect of PARPi when administered with vaccine (FIG. 12E). In addition, PARP inhibition increased vaccine-induced total and antigen-specific effector (T_{EM}; CD62L⁻CD44⁺) and central memory CD8 T cells (T_{CM}; CD44⁺CD62L⁺) in the TME (FIGS. 1F and 12F-12I). Importantly, it was observed that PARPi treatment in combination with vaccine led to the appearance of a distinct population of CD8 T cells that expressed high levels of both CD44 and CD62L (CD44^{hi}CD62L^{hi}) within the T_{CM} compartment in the TME (FIG. 1G). Interestingly, despite the fact that inhibition of PARP increased the function of CD8 T cells, PARPi showed a significant decrease in proliferation as determined by Ki67 expression (FIGS. 1H and 12J) in line with a previous report that showed that knockout (KO) of PARP1 results in their reduced proliferation (Saenz et al., BMC Genomics 9:171, 2008). This reduction in proliferation may drive CD8 T cells towards memory as shown previously (Verma, et al., Nat Immunol. 22(1): 53-66, 2021; Kretschmer et al., Nat Commun 11(1): 113, 2020).

Together, the above data indicate that inhibition of PARP enhances infiltration of the TME with highly functional antigen-specific CD8⁺ T cells as well as T_{EM} and T_{CM} cells, leading to potent anti-tumor effects.

### Example 3: PARPi Leads to the Development of a Novel Subpopulation of Memory CD8 T Cells

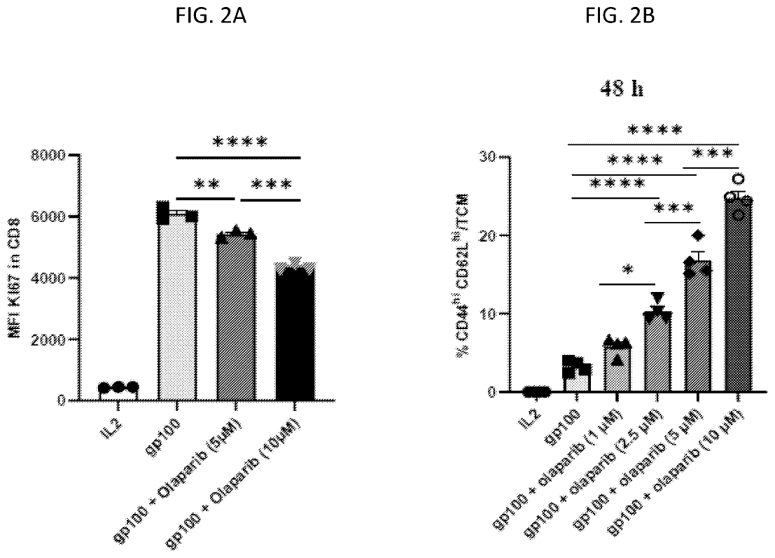

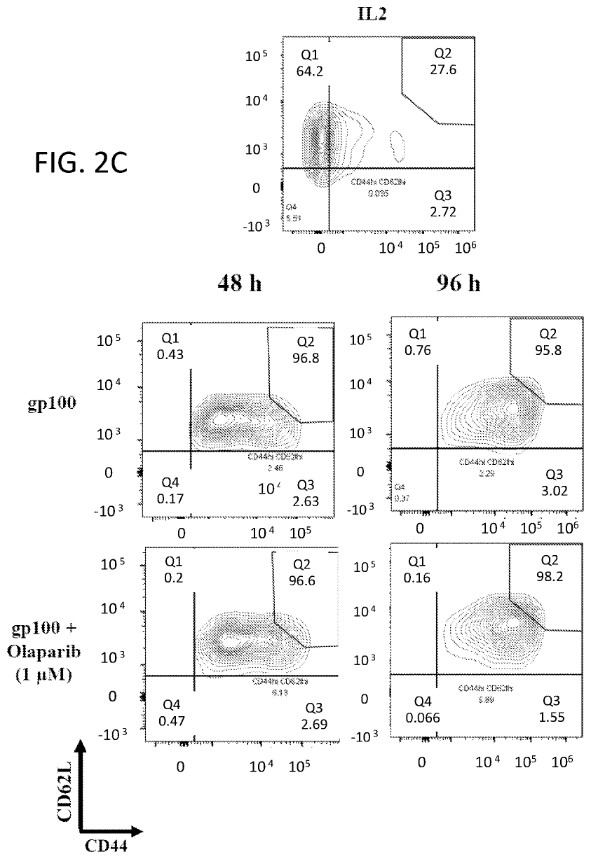

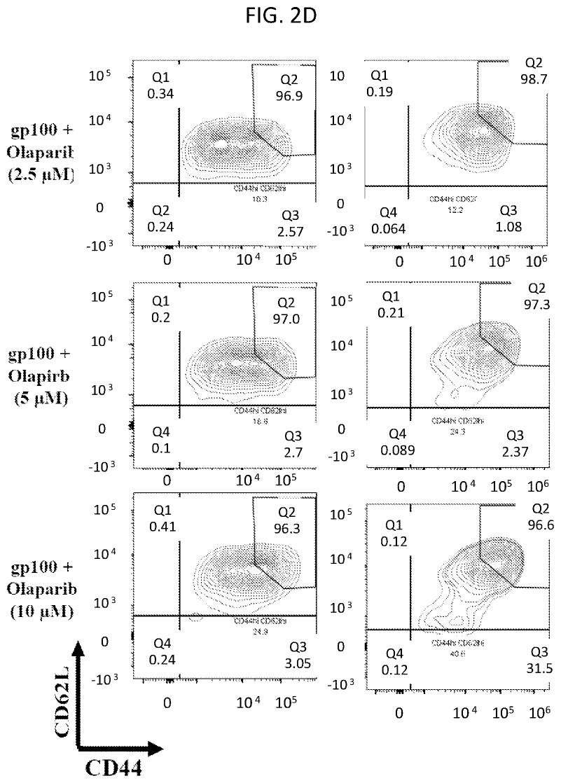

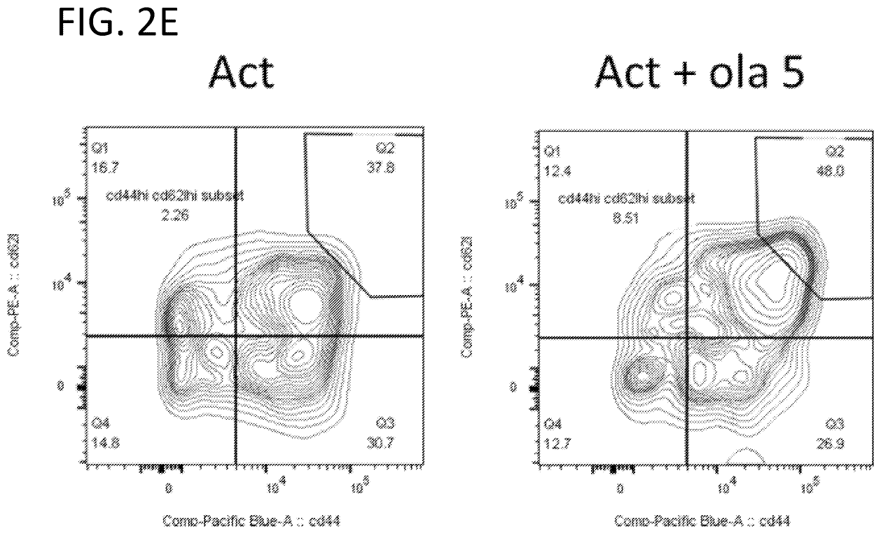

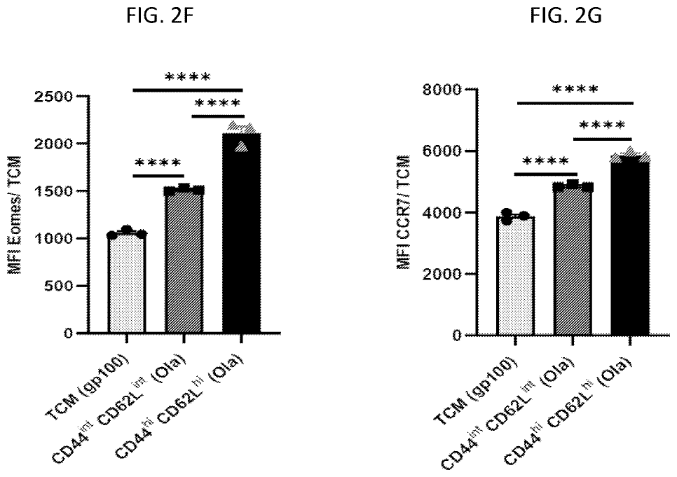

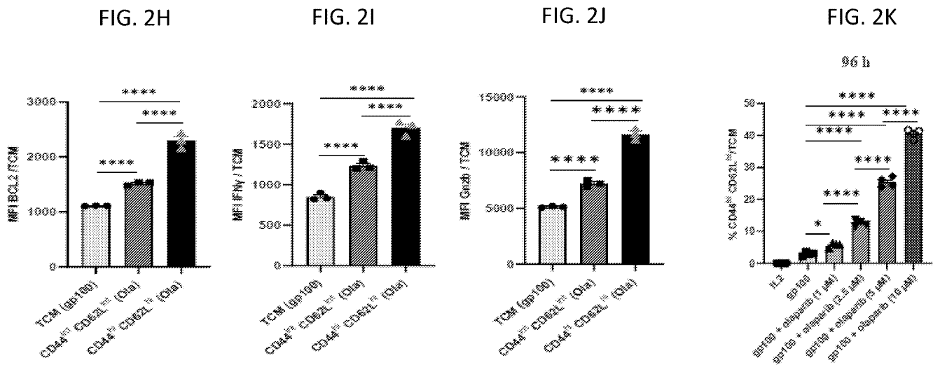

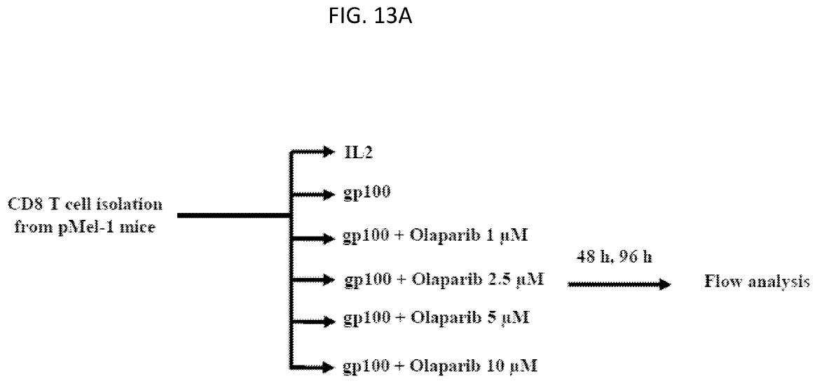

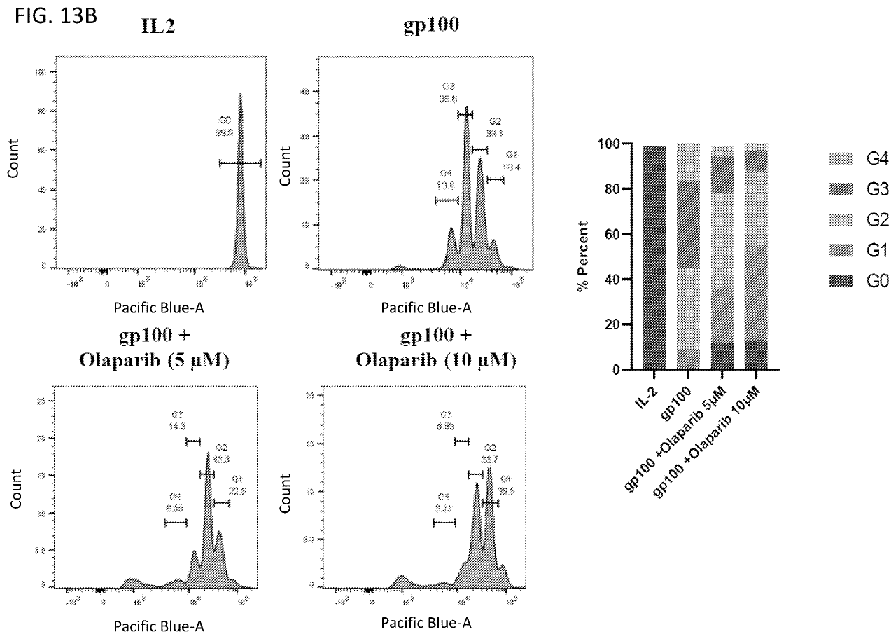

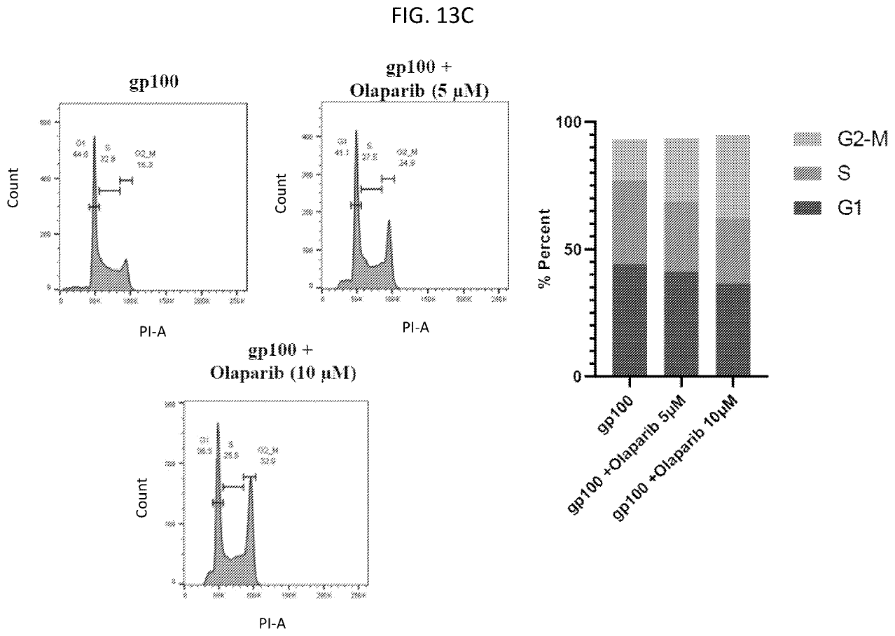

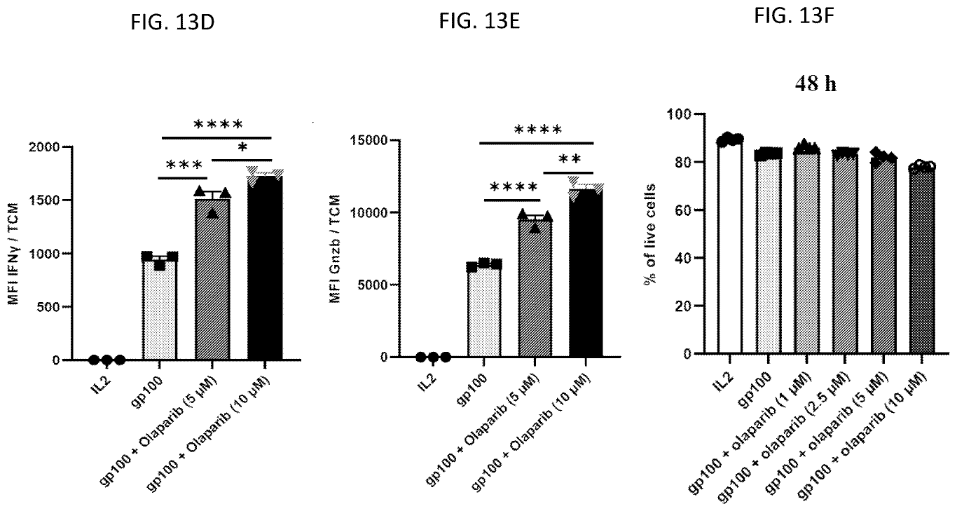

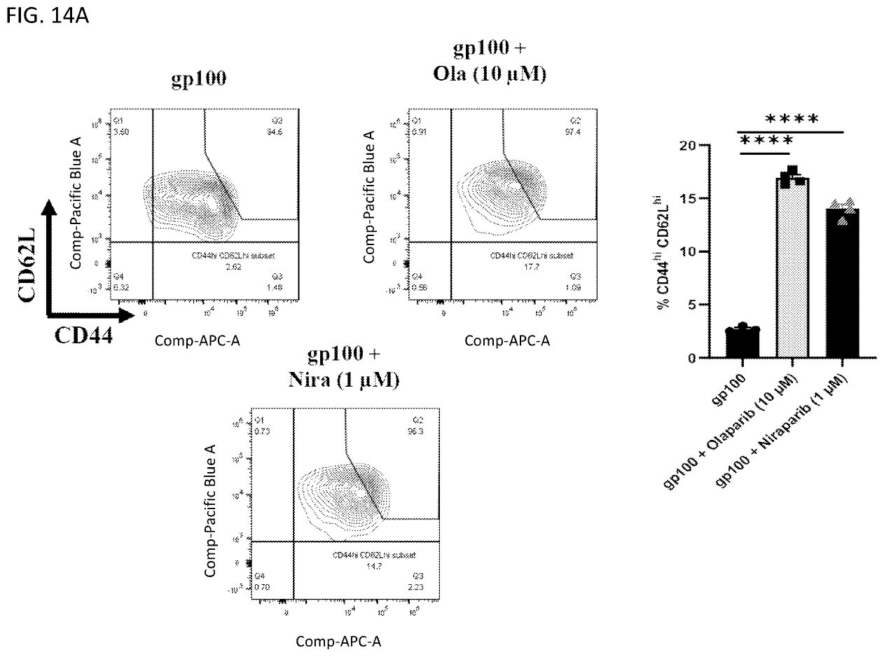

Since it was found that the anti-tumor effect of PARPi is mediated by CD8 T cells, additional studies were performed to understand the direct effect of inhibiting PARP on activated CD8 T cells. CD8 T cells obtained from TCR engineered pMel-1 mice were activated with gp100 antigen and their phenotype was checked when treated with different concentrations of PARPi (FIG. 13A). Interestingly, similar to what was observed in vivo in the TME, PARP inhibition significantly reduced the proliferation capacity (FIG. 2A). Also in line with these findings, these cells arrested in the G2/M phase (FIGS. 13B-13C). Furthermore, similar to the in vivo observations, there was an increase in the percentage of T_{CM} CD8 T cell population with higher expression of CD62L and CD44 (CD44^{hi}CD62L^{hi}) in a concentration-dependent manner at 48 h of treatment (FIGS. 2B-2D). The generation of the CD44^{hi}CD62L^{hi} population was also seen in WT CD8 T cells activated with anti-CD3/CD28 in the presence of PARPi (FIG. 2E). In addition to CD62L and CD44, PARPi significantly increased the expression of other CD8 T cell memory markers including Eomes, CCR7, and Bcl2 (an anti-apoptotic marker enhanced in memory T cells (Choi et al., Immune Netw 20(3):e20, 2020)) in CD44^{hi}CD62L^{hi} population compared to T_{CM} (FIGS. 2G-21). Since it was found that PARPi enhanced CD8 T cells cytotoxic function in vivo, IFNγ and Gzm B expression on activated CD8 T cells after PARPi in vitro was evaluated. The results showed a significant increase in their expression compared to gp100 alone (FIGS. 13D-13E). Furthermore, PARPi-induced CD44^{hi}CD62L^{hi} cells expressed significantly higher IFNγ and Gzm B compared to T_{CM} (FIGS. 2I-2J). Accordingly, these cells represent a memory cell population with potentially enhanced function. Interestingly, PARPi also induced a cell population that had higher expression of CD62L, CD44, Eomes, CCR7, Bcl2 as well as IFNγ and Gzm B than the gp100-induced T_{CM} but expression of these markers/molecules was significantly less than CD44^{hi}CD62L^{hi} cells (FIGS. 2C-2D and 2F-2J). This suggested that these cells are likely to be cells in-transition and thus were considered intermediate and were labeled as CD44^{int}CD62L^{int}. The phenotyping at 96 h after PARPi treatment confirmed that CD44^{int}CD62L^{int} are the transitioning intermediate population that shifted more toward CD44^{hi}CD62L^{hi} population at later time points (FIGS. 2C-2D, 2K). However, further phenotyping and functionality was conducted in pMel-1 CD8 T cells at the 48 h time point since PARPi did not show any significant toxicity at this timepoint (FIG. 13F).

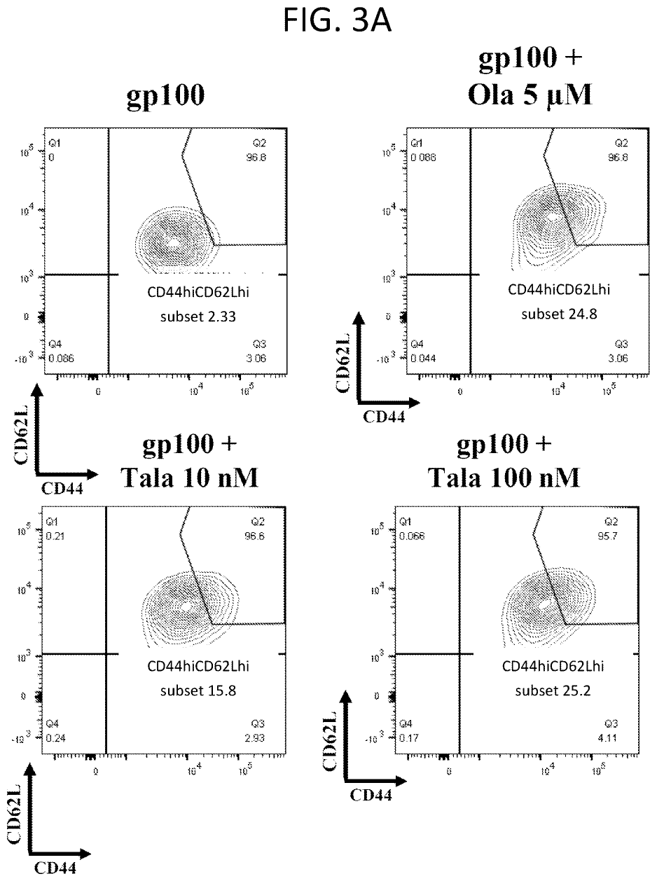

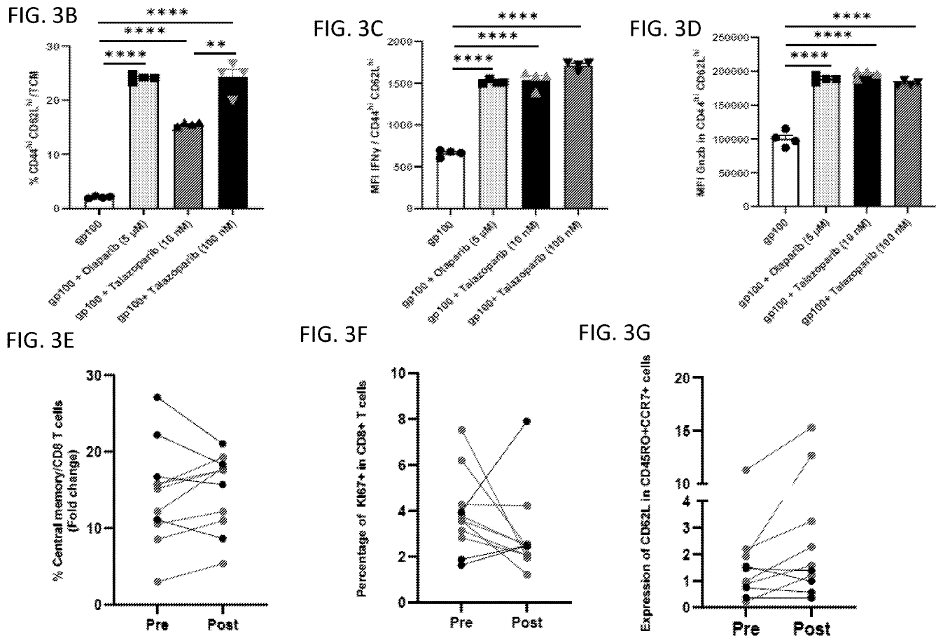

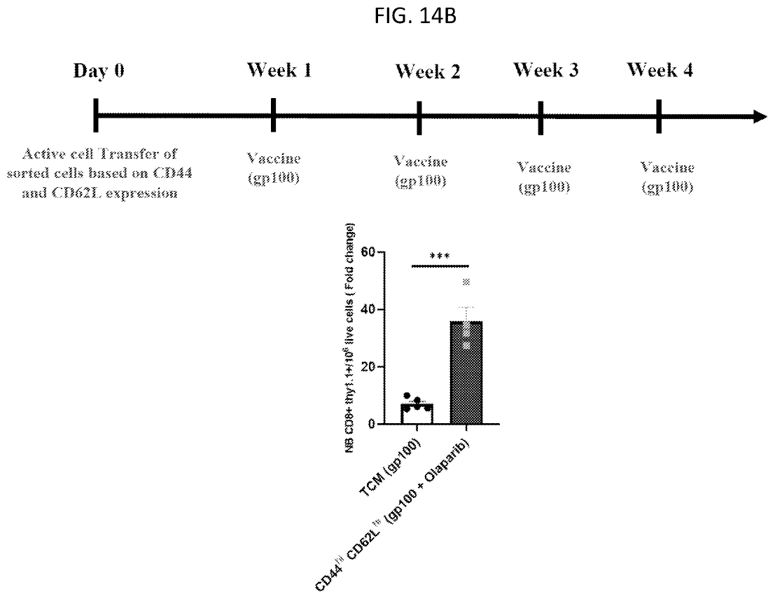

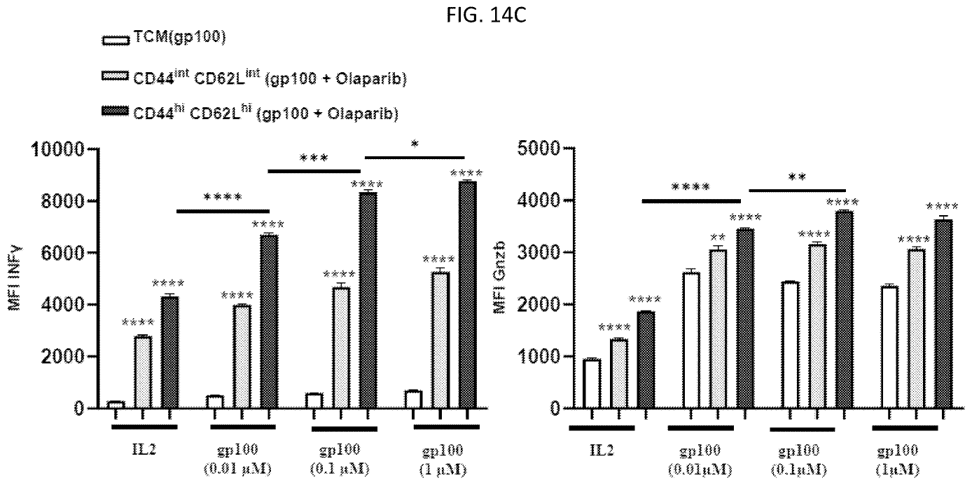

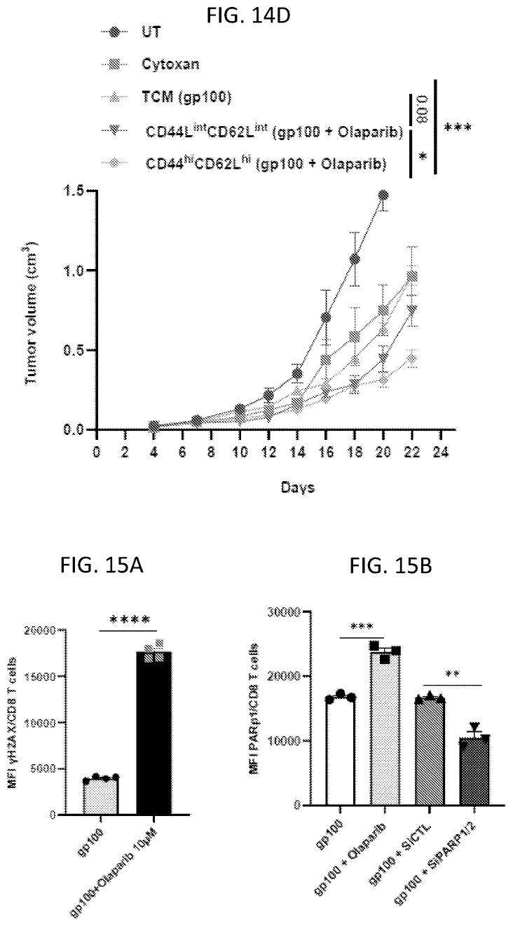

Finally, it was confirmed that the effect of PARPi on the generation of enhanced memory phenotype (CD44^{hi}CD62L^{hi}) is not olaparib specific. For this, two other PARP inhibitors, Talazoparib and Niraparib (Lord and Ashworth, Science 355 (6330): 1152-1158, 2017), were tested. Similar to olaparib, Talazoparib and Niraparib significantly increased the percentage of CD44^{hi}CD62L^{hi} CD8 T cell population after activation (FIGS. 3A-3B and 14A). Furthermore, expression of IFNγ and Gzm B was also significantly increased in these cells following treatment with Talazoparib, similar to olaparib (FIGS. 3C-3D).

Similar to the observations in vitro and in mouse tumor models, in a cohort of ovarian cancer patients treated with olaparib plus anti-PDL1 (Durvalumab), there was an increase in T_{CM} CD8 T cells in 63% of patients (FIG. 3E). This was also associated with decrease in the frequency of CD8 T cells expressing Ki67⁺CD8 T cells (FIG. 3F). In addition, CD62L expression was also increased in T_{CM} CD8 T cells of some patients (FIG. 3G). This also demonstrated the role of PARPi in CD8 memory T cell differentiation in human in an in vivo setting.

### Example 4: CD62L^{hi}CD44^{hi} Cells Generated by PARPi are Super-Central Memory (T_{CM}^{sup}) CD8 T Cells with Higher Recall Potency and Anti-Tumor Effect

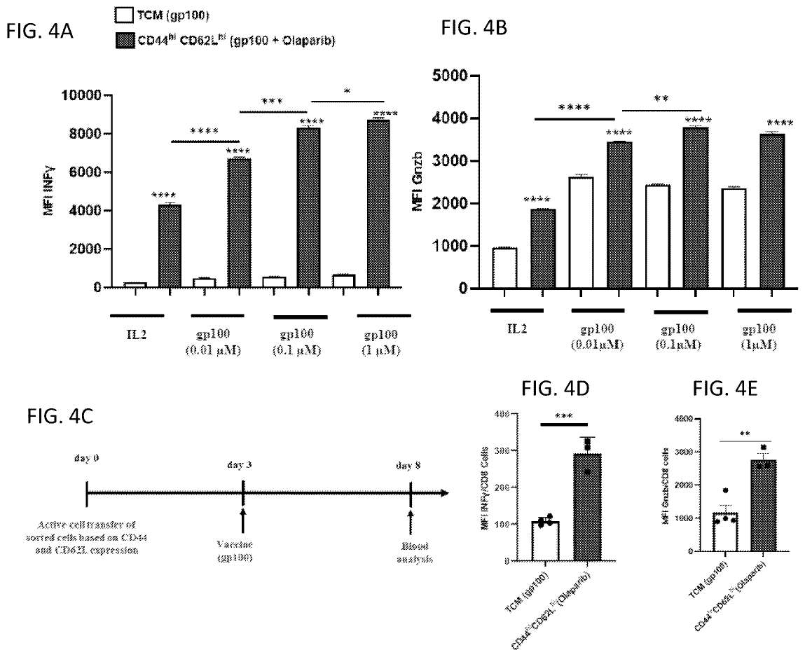

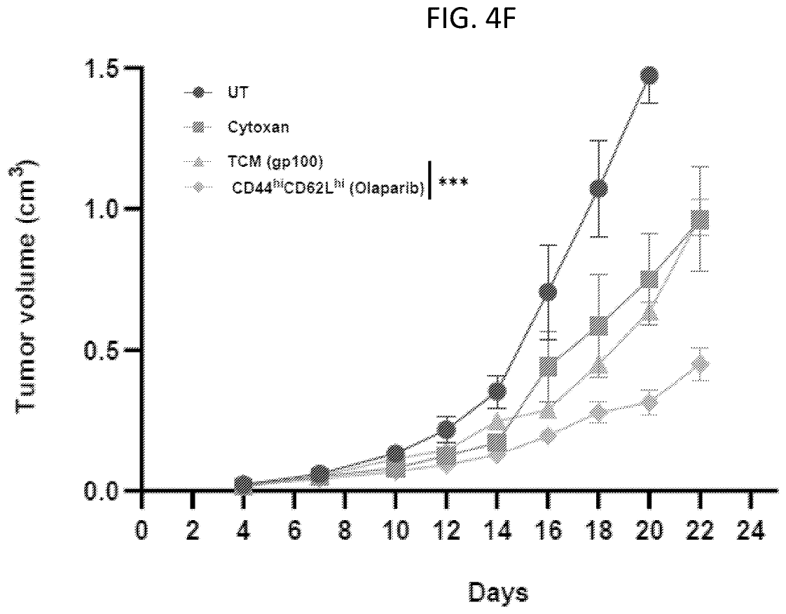

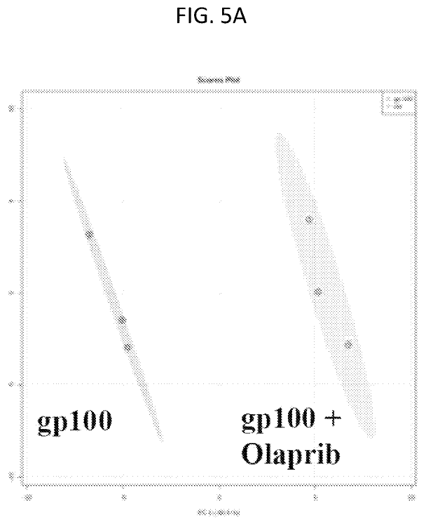

Since the studies described in the Examples above demonstrated that PARPi generates a potentially enhanced memory phenotype with higher expression of CD62L, CD44, and other memory and cytotoxic markers, a study was performed to determine whether this phenotype possesses higher level of memory capability. Accordingly, the recall response of the CD44^{hi}CD62L^{hi} T_{CM} CD8 subpopulation induced by PARPi was first tested. After the first activation with gp100 in the presence of PARPi, CD44^{hi}CD62L^{hi} population was sorted and re-challenged with different antigen concentrations. Indeed, PARPi-induced CD44^{hi}CD62L^{hi} memory CD8 T cells showed a 10-15 times higher INFγ recall response capability when compared to T_{CM} generated by antigen alone (T_{CM}-generated by gp100) (FIG. 4A). A similar trend was observed with Gzm B (FIG. 4B). To further confirm the higher recall capacity of the CD44^{hi}CD62L^{hi} cells, the recall of these cells was tested in vivo. In vitro PARPi generated CD44^{hi}CD62L^{hi} pMel-1 CD8 T cells were adoptively transferred into C57BL/6 naïve mice and their activation status in response to gp100 peptide vaccine was assessed (FIG. 4C). The CD62L^{hi}CD44^{hi} population had significantly higher INFγ and Gzm B expression compared to the normal T_{CM} from gp 100 group (FIGS. 4D-4E). Since one of the mechanisms of recall capacity is also increased proliferation (Barski et al., Sci Rep 7:39785, 2017), the proliferation of adoptively transferred PARPi-induced CD44^{hi}CD62L^{hi} cells and gp100-induced T_{CM} was tested in C57/BL6 mice after challenging them with gp100 vaccine weekly for 4 weeks. Interestingly, the CD44^{hi}CD62L^{hi} population had a higher proliferative ability when compared to T_{CM}-induced after gp100 stimulation by at least by 5-fold (FIG. 14B). Together, these data demonstrate that inhibition of PARP triggers the generation of a superior T_{CM} population that is named herein as T_{CM}^{sup} cells with greater recall and anti-tumor effects.

In addition, PARPi-induced T_{CM} population expressing CD44^{int}(CD62L^{int} also demonstrated a significantly higher INFγ and Gzm B recall activity compared to gp100-induced T_{CM}, albeit less than the CD44^{hi}CD62L^{hi} cells. Furthermore, the expression of these proteins plateaued at a 10 times lower concentration (0.01 μM) when compared to the CD44^{hi}CD62L^{hi} population (FIG. 14C). Similar data were obtained when these cells were tested in vivo, where CD44^{int}CD62L^{int} cells demonstrated significantly higher INFγ recall compared to gp100-induced T_{CM} but lesser than CD44^{hi}CD62L^{hi} cells (FIG. 14D). This further indicates that CD44^{int}CD62L^{int} could represent a transitional state of the CD44^{hi}CD62L^{hi} cells.

The therapeutic efficacy of these CD44^{hi}CD62L^{hi} cells generated by inhibiting PARP were tested in adoptive transfer experiment in tumor-bearing mice. The results showed that C57BL/6 B16F10 tumor-bearing mice that received PARPi-induced CD44^{hi}CD62L^{hi} T_{CM} had significantly more potent tumor growth inhibition compared to mice that received gp100-induced T_{CM} CD8 T cells (FIG. 4F). This anti-tumor effect was also observed with PARPi-induced CD44^{int}CD62L^{int} cells, further confirming that the T_{CM} cells generated after PARPi are functionally superior to the T_{CM} generated by gp100.

These findings highlight the ability of PARPi to differentiate activated T cells into a unique memory phenotype, T_{CM}^{sup}, which is highly functional compared to the memory population generated upon activation of CD8 T cells.

### Example 5: Inhibition of Enzymatic Activity of PARP and its Trapping by PARPi Results in Metabolic Reprograming and a Transcriptional Switch in CD8 T Cells, Leading to Generation of T_{CM}^{sup} Cells

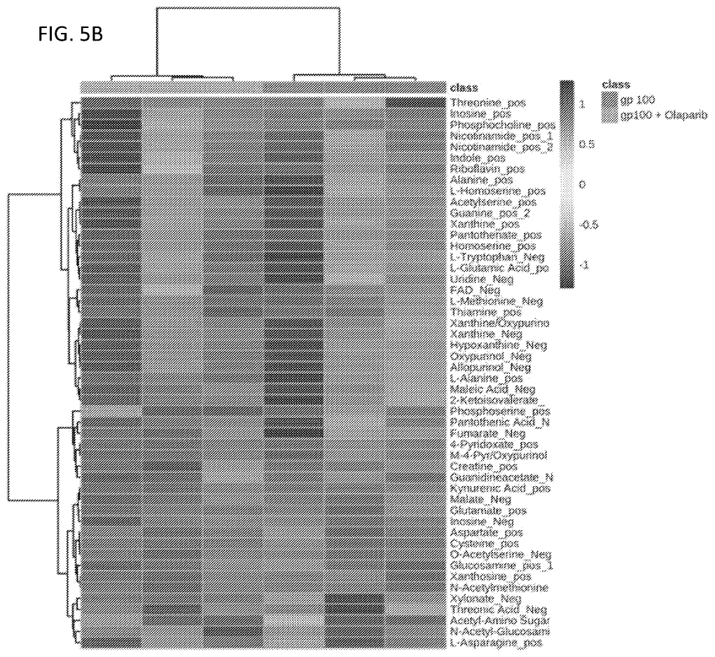

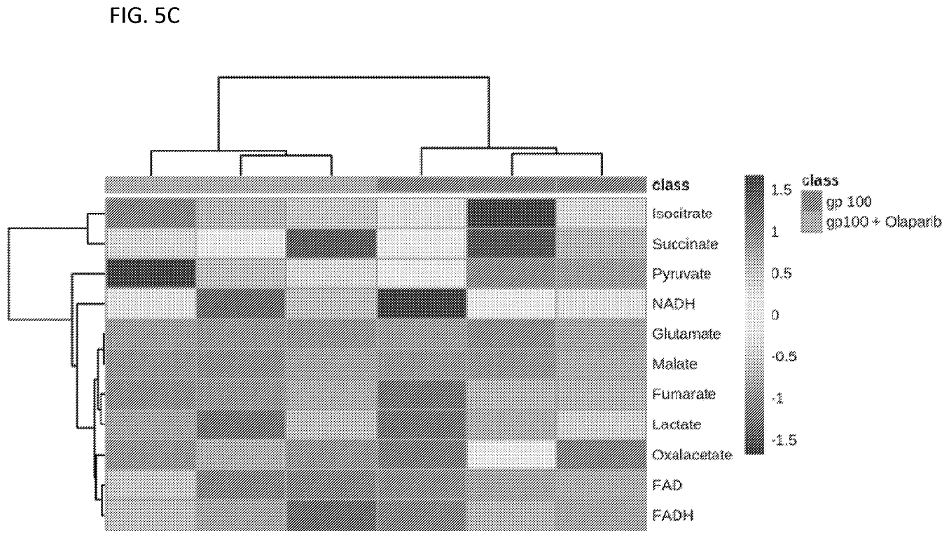

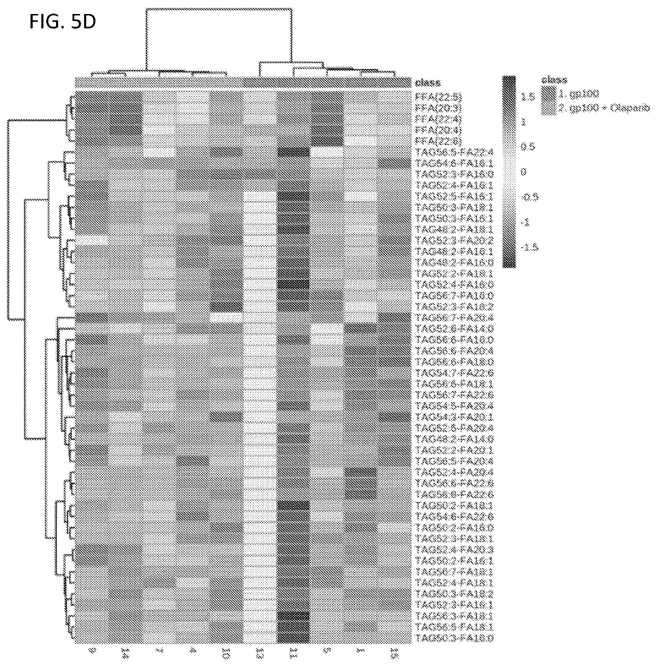

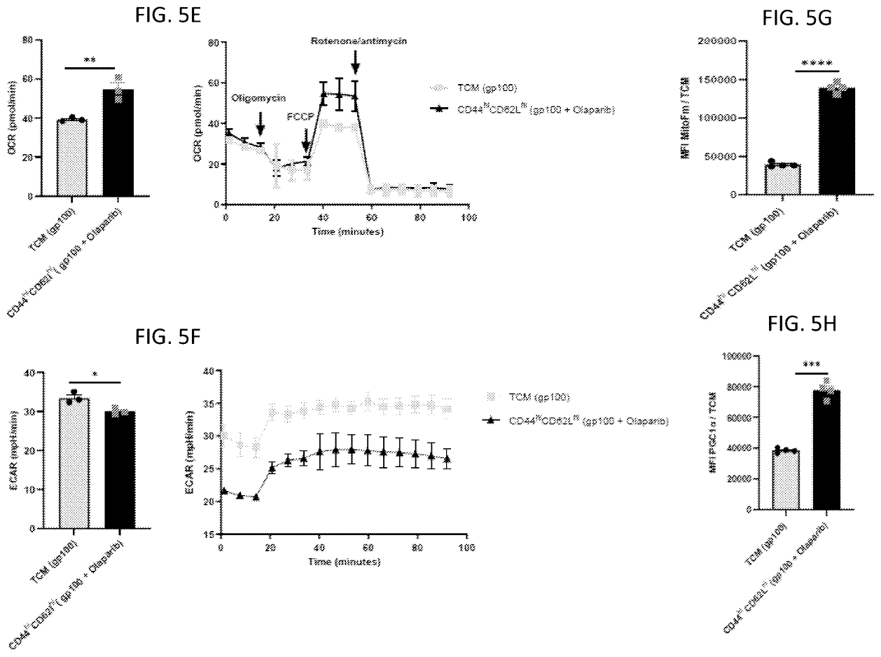

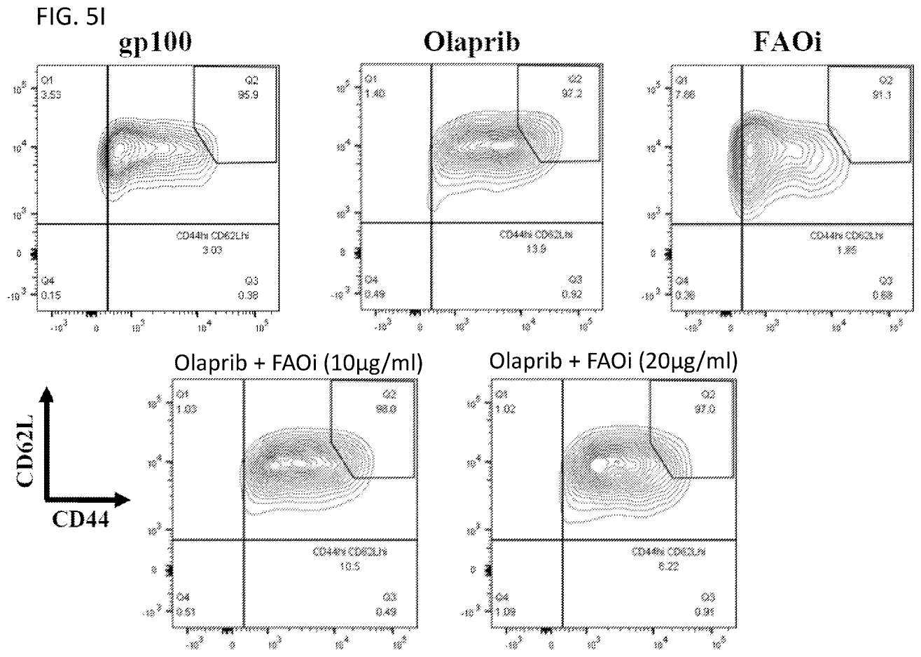

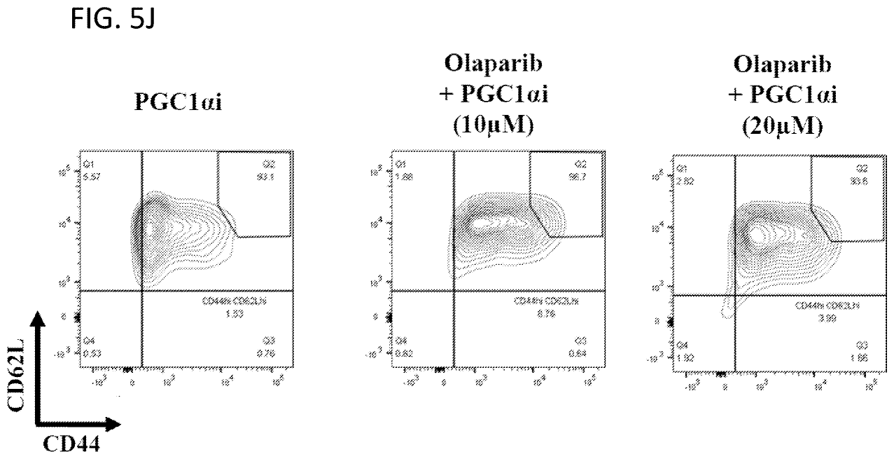

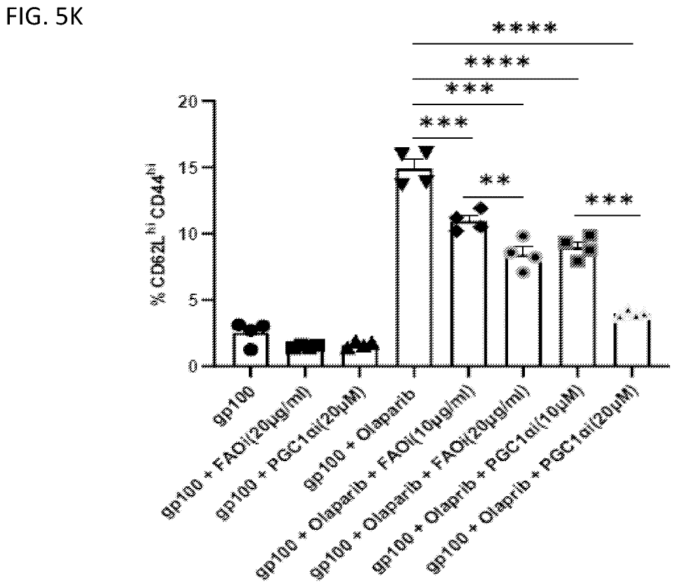

As outlined above, PARPi-induced CD44^{hi}CD62L^{hi} CD8 T cells (T_{CM}^{sup}) demonstrated greater immune recall capacity and greater anti-tumor effect. CD8 T_{CM} are highly dependent on FAO and rely on cell intrinsic lysosomal acid lipase (LAL)-mediated lipolysis of triacylglycerides (TAGs) to fuel FAO (Zhang and Romero, Trends Mol Med 24(1):30-48, 2018). Therefore, it was first tested whether PARPi affects the metabolic fitness of CD8 T cells, leading to the generation of T_{CM}^{sup}. For this, pMel-1 CD8 T cells were in vitro activated with or without PARPi for 48 h and deep metabolomics and lipidomics analysis were performed. The principal component analysis (PCA) from metabolomics data showed that PARPi-treated cells were highly distinct from antigen (gp100)-activated cells (FIG. 5A). Interestingly, an unsupervised analysis demonstrated that all of the top 50 metabolites that were differentially expressed between the PARPi-treated and gp100 activated CD8 T cells were increased in the PARPi-treated group (FIG. 5B). Among these metabolites, most of the metabolites related to tricarboxylic acid (TCA) cycle such as fumarate, malate, glutamate, FAD were significantly increased in PARPi-treated CD8 T cells compared to gp100 activated cells (FIG. 5C). This data is consistent with the role of oxidative phosphorylation (OXPHOS) in memory CD8 T cells (Zhang and Romero, Trends Mol Med 24(1):30-48, 2018). Furthermore, the results showed an increased level of TAGs in PARPi-treated CD8 T cells compared to the gp 100 activated cells in lipidomic analysis (FIG. 5D), which is another important characteristic of memory CD8 T cells (Zhang and Romero, Trends Mol Med 24 (1): 30-48, 2018). Since both metabolomic and lipidomic analysis demonstrated that PARPi enhances the metabolic fitness of CD8 T cells, the glycolytic and OXPHOS capacity in PARPi-induced T_{CM}^{sup} cells using were measured using Seahorse assays. In line with enhanced TCA metabolites shown by metabolomics (FIG. 5b-c), a significant increase in oxygen consumption rate (OCR; OXPHOS) and a decrease in extracellular acidification rate (ECAR; Glycolysis) were observed in PARPi-induced T_{CM}^{sup} cells compared to gp100-induced T_{CM} cells (FIGS. 5E-5F), further suggesting their enhanced metabolic fitness. In addition, PARPi significantly led to increase in the mitochondrial mass in T_{CM}^{sup} cells (FIG. 5G). This effect can be explained by an increase in the PARPi-mediated peroxisome proliferator-activated receptor gamma coactivator-1 alpha (PGC1α) expression (FIG. 5H), one of the important regulators of mitochondrial biogenesis (Austin and St-Pierre, J Cell Sci 125 (Pt 21): 4963-4971, 2021). To confirm the role of PGC1α or FAO in the induction of T_{CM}^{sup} cells, it was found that the inhibition of either PGC1α or FAO indeed reduces PARPi-mediated induction of T_{CM}^{sup} cells (FIGS. 51-5K). Together, these results showing a decreased dependence of PARPi-treated CD8 T cells on glycolysis with increased utilization of FAO and OXPHOS for their metabolic needs indicate that PARPi-induced metabolic switch toward FAO and OXPHOS, leads to metabolic reprograming of CD8 T cell and the generation of T_{CM}^{sup} cells.

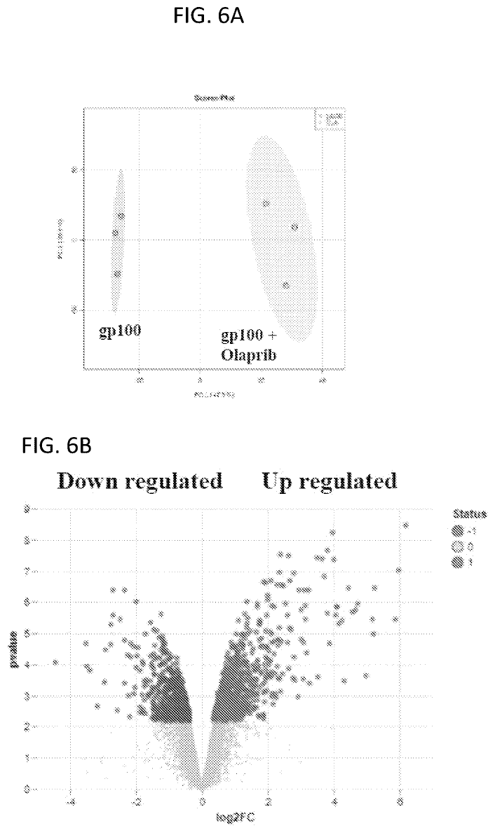

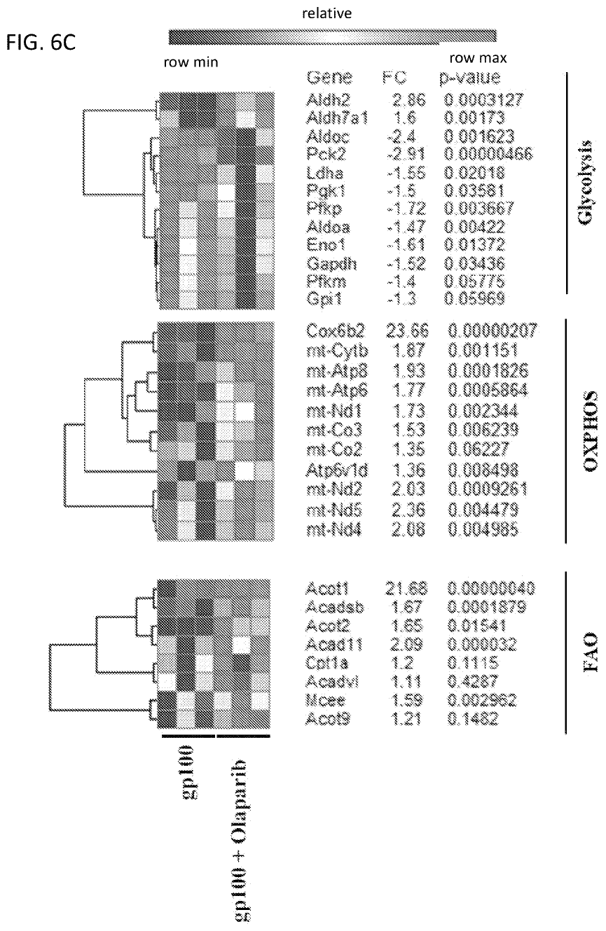

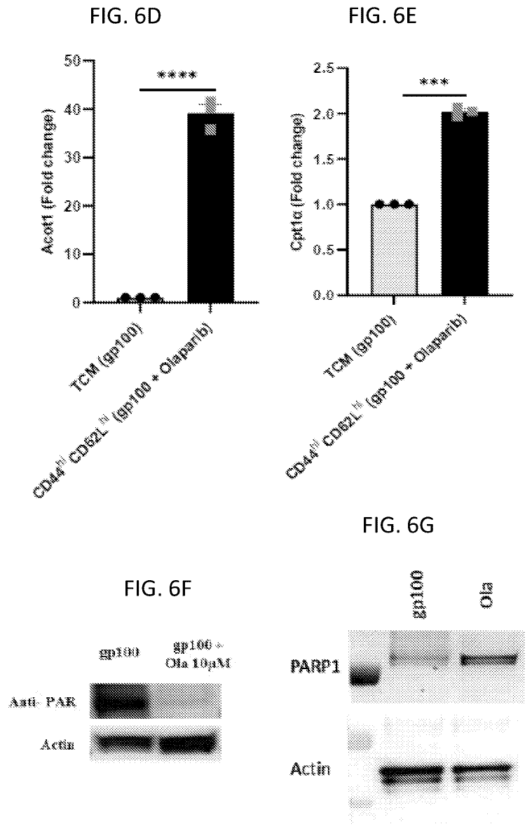

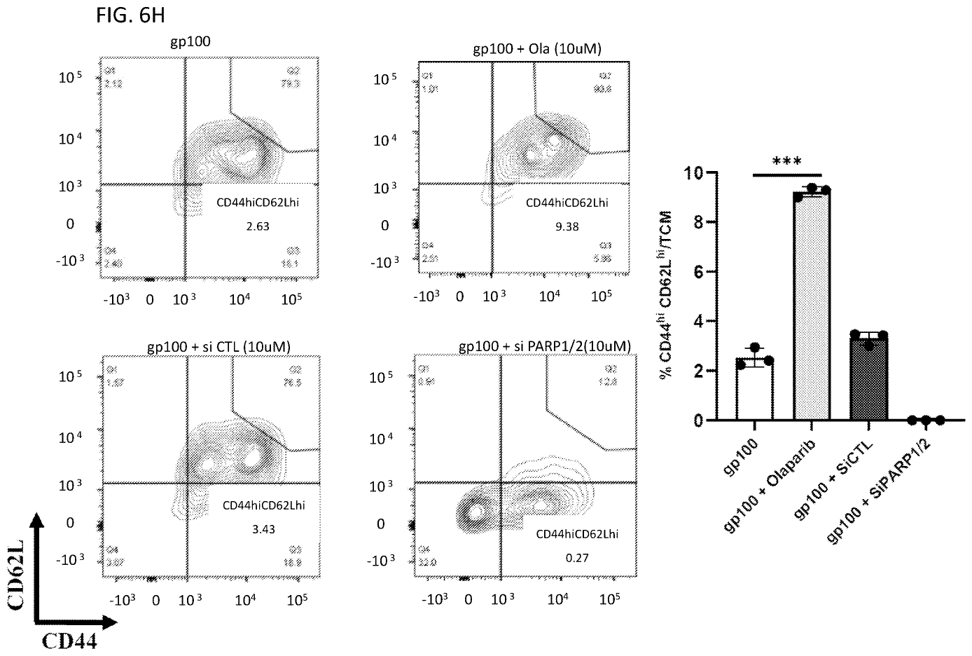

Based on the transcriptional activity of PARP in regulating several genes related to T cell activation and metabolism upon antigen stimulation (Saenz et al., BMC Genomics 9:171, 2008), it was checked whether the PARPi-mediated metabolic reprograming in CD8 T cells was modulated at the transcriptional level. For this, RNA-sequencing analysis of gp100-activated pMel-1 CD8 T cells in the presence or absence of PARPi was performed. PCA plot demonstrated that PARPi-treated CD8 T cells had a very distinct cluster of transcriptomes compared to gp 100-treated cells (FIG. 6A). Furthermore, in concordance with the PARPi-mediated metabolic reprograming described above, a significant differential expression of genes (DEGs) was found between the two populations (FIG. 6B). As expected, several genes related to the glycolysis pathway (Eno1. Aldoc, Pgk1. Gapdh) were significantly downregulated while several genes related to OXPHOS and FAO were significantly upregulated, especially Acot1 and Cpt1α (a key enzyme of FAO) in PARPi-treated CD8 T cells (FIG. 6C). To confirm that PARPi-induced T_{CM}^{sup} have high expression of FAO-related genes, the level of Acot1 and Cpt1α were determined by real time polymerase chain reaction (RT-PCR). T_{CM}^{sup} were prepared from pMel-1 CD8 T cells with PARPi, as outlined above. As expected, the expression of Acot1 and Cpt1α was significantly higher in T_{CM}^{sup} cells compared to gp100-induced T_{CM} cells (FIGS. 6D-6E).

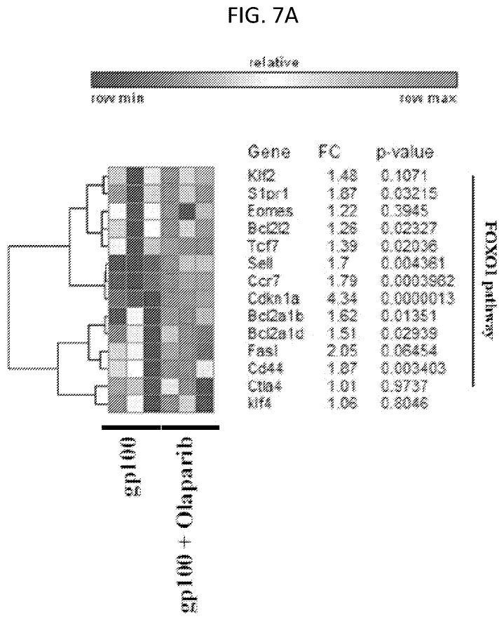

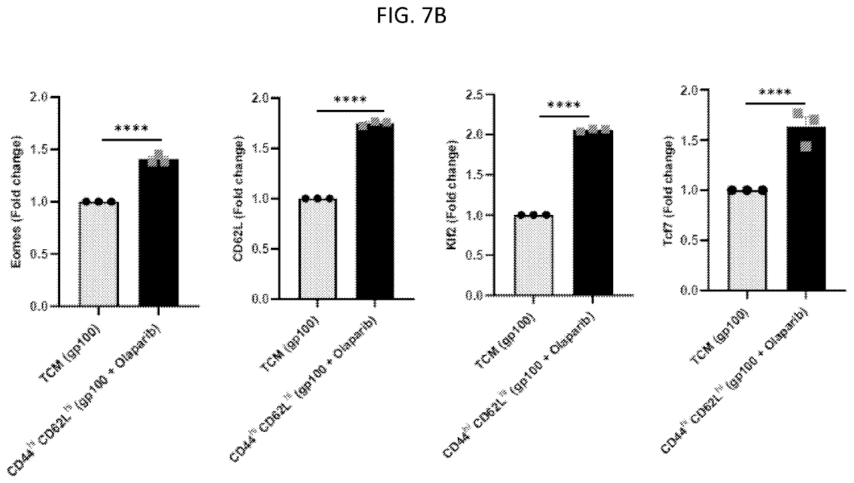

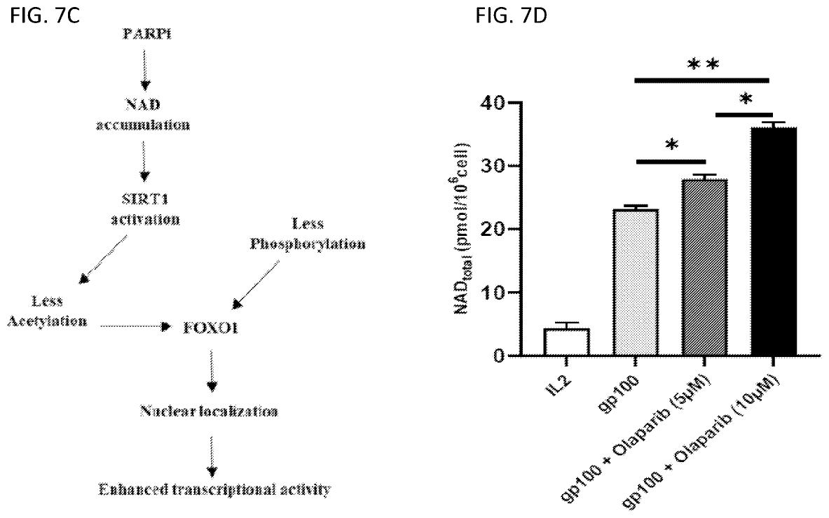

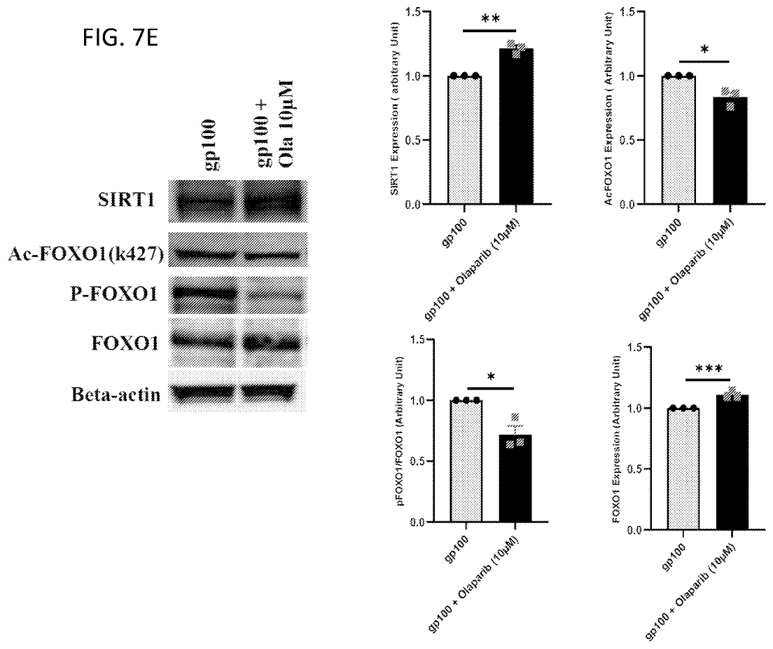

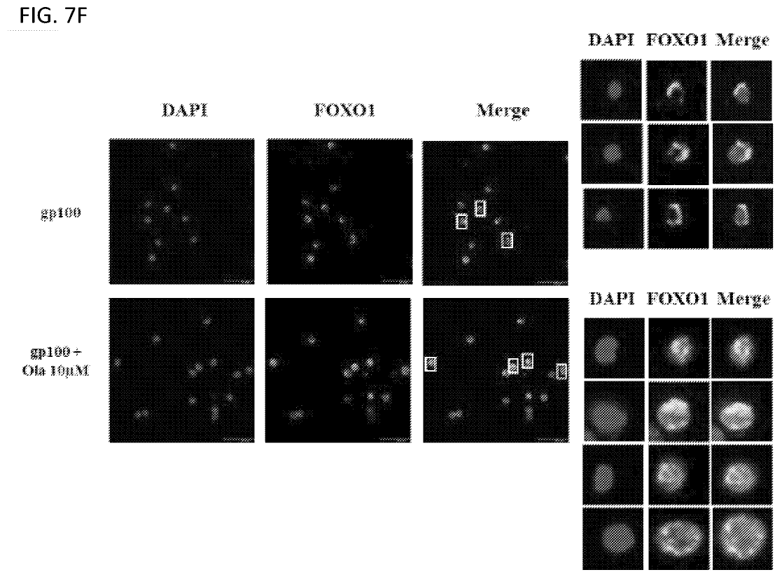

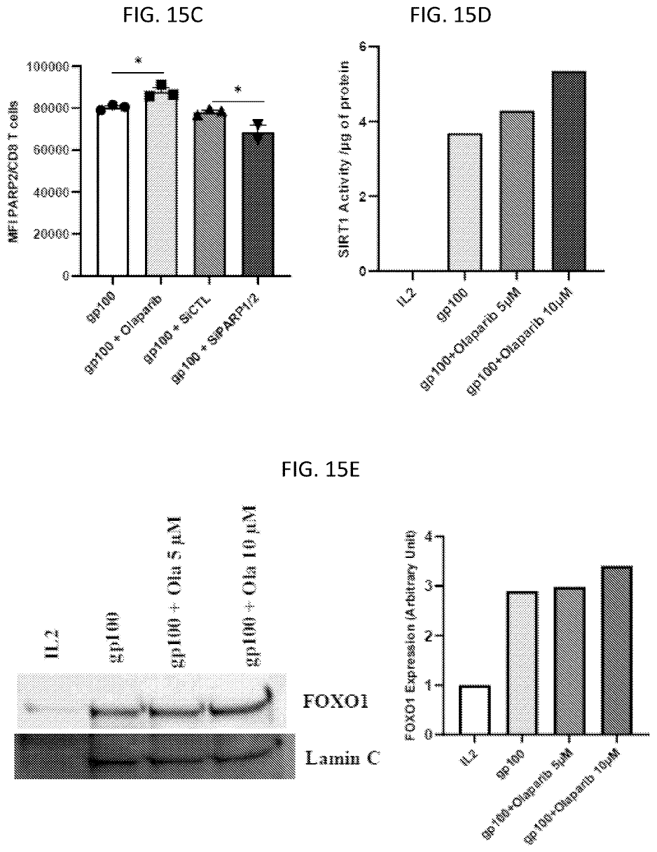

PARP possesses transcriptional regulatory and chromatin remodeling functions in addition to its enzymatic role in DDR (Krishnakumar and Kraus, Mol Cell 39 (1): 8-24, 2010). The present studies determined that inhibition of PARP completely abolished the protein enzymatic activity as seen by a significant decrease in PAR expression (FIG. 6F) and an increase in the level of γH2AX (FIG. 15A). Accordingly, it was hypothesized that additional mechanisms that are independent of the enzymatic activity of PARP may be involved in generating T_{CM}^{sup} after PARP inhibition. One such potential mechanism is the increase in the trapping of the PARP protein in the nucleus, leading to its enhanced transcriptional activity. Indeed, it was found that inhibition of PARP results in a significant increase in PARP1 and PARP2 expression in the nucleus of CD8 T cells (FIGS. 6G and 15B-15C), suggesting that trapping may be required for generation of T_{CM}^{sup} cells. To further confirm the relation between trapping and transcriptional modulation by PARPi, PARP1/2 was knocked down to decrease the level of trapped PARP protein in the nucleus and the generation of T_{CM}^{sup} was evaluated (Murai et al., Cancer Res 72 (21): 5588-5599, 2012). The data showed that contrary to inhibition of PARP, downregulation of PARP1/2 by siRNA did not generate T_{CM}^{sup} (FIGS. 6H and 15B-15C). Together, this suggests that trapping of PARP in the nucleus is important for PARPi-mediated transcriptional changes that results in the generation of T_{CM}^{sup}. Indeed, similar to the observed increase in the memory markers at protein level, RNA-seq data showed significant increase in the expression of several genes related to memory (Sell or Cd62l, Bcl2, Ccr7, Slpr1, Cd44, Tcf7, Klf2, and Eomes) after PARPi treatment in CD8 T cells (FIG. 7A), confirming a crucial role of PARP-mediated transcriptional activity in the generation of T_{CM}^{sup}.

### Example 6: PARPi Mediated-T_{CM}^{sup} are Generated Through SIRT1/FOXO1 Pathway Activation

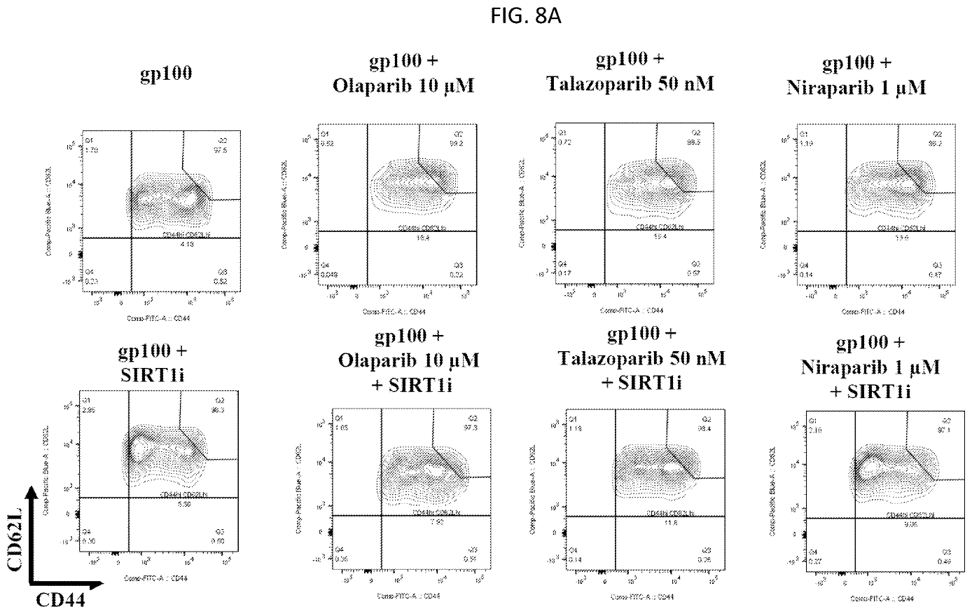

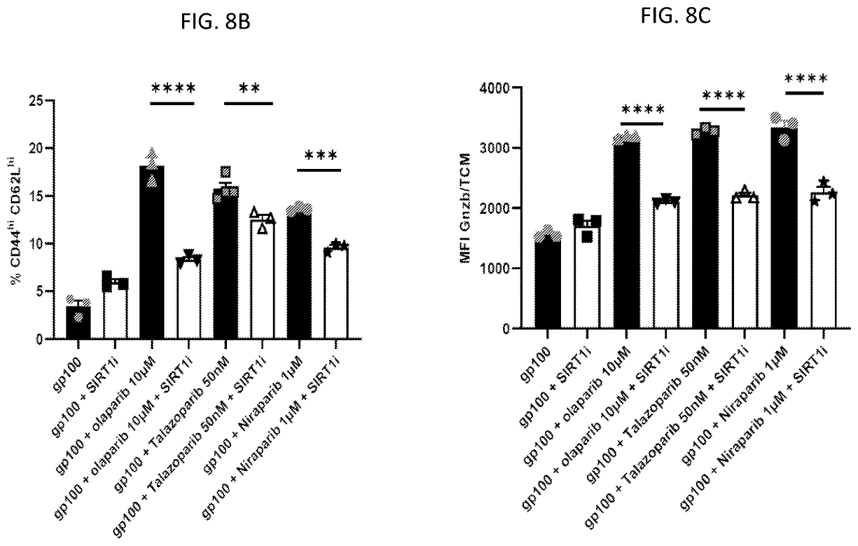

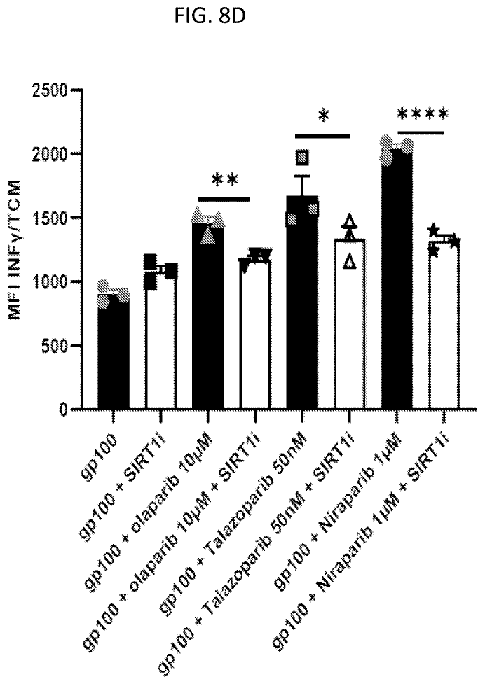

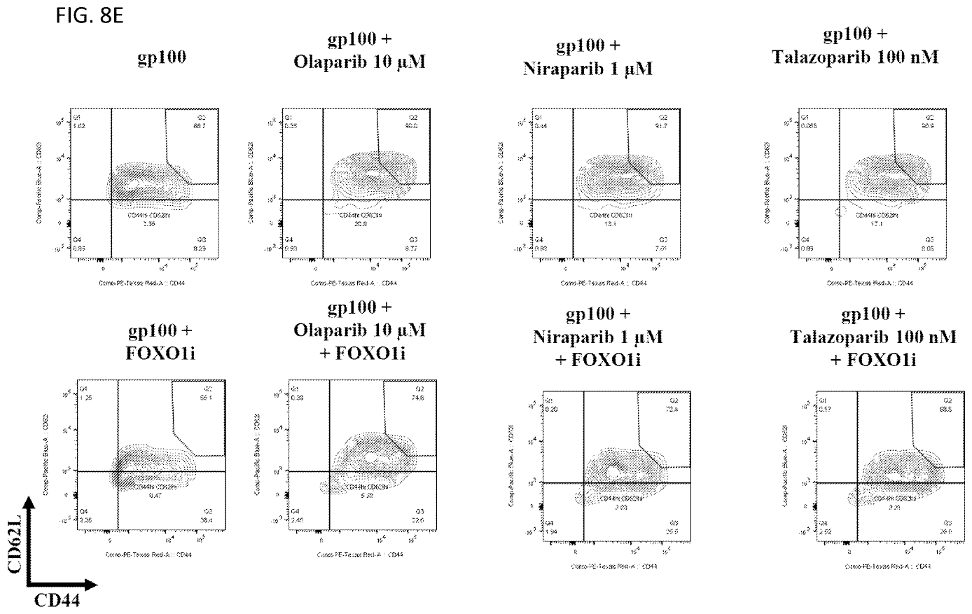

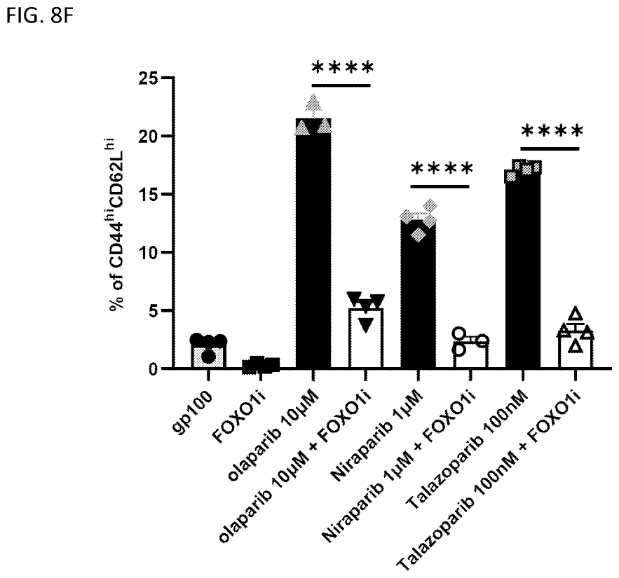

As described above, PARP inhibition results in a significant increase in the expression of several memory related genes in CD8 T cells. Therefore, further studies were conducted to identify the potential signaling pathways involved in the generation of PARPi-mediated T_{CM}^{sup}. The expression of some of the key memory genes Cd62l and others (Klf2, Tcf7, Ccr7) upregulated after PARP inhibition are regulated by FOXO1 (Delpoux et al., Proc Natl Acad Sci USA 114(42):E8865-E8874, 2017; Gray et al., Immunity 46(4):596-608, 2017; Ouyang et al., Immunity 30(3):358-371, 2009). RT-PCR analysis confirmed that these genes were upregulated in T_{CM}^{sup} cells compared to gp100-induced T_{CM} cells (FIG. 7B). Further, FOXO1 is regulated by SIRT1, a deacetylase enzyme that antagonizes PARP1, as both utilize the NAD⁺ for their activity (Chung and Joe, Integr Med Res 3(4):198-203, 2014). Based on this, it was hypothesized that inhibition of PARP could lead to accumulation of NAD⁺, increasing SIRT-1/FOXO1 signaling, resulting in generation of T_{CM}^{sup} population (FIG. 7C). To test this, the level of NAD⁺ and expression of SIRT-1 in activated pMel-1 CD8 T cells after PARP inhibition was determined. PARPi significantly enhanced the accumulation of NAD (FIG. 7D), leading to increased SIRT-1 expression and activity (FIGS. 7E and 15D). It was next checked if the increase in SIRT-1 activity results in FOXO1 deacetylation. The results showed a decrease in FOXO1 acetylation in PARPi-treated CD8 T cells (FIG. 7E). Since acetylation of FOXO1 is known to increase its phosphorylation, resulting in inhibition of its nuclear translocation and transcriptional activity (Matsuzaki et al., Proc Natl Acad Sci USA 102(32):11278-11283, 2005), it was hypothesized that SIRT-1-mediated FOXO1 deacetylation would decrease its phosphorylation, trapping it in the nucleus and enhancing its transcriptional activity. Accordingly, the level of total and phosphorylated FOXO1 was checked and the results showed a significant reduction in FOXO1 phosphorylation and its increased expression after PARPi treatment of CD8 T cells in total lysate (FIG. 7E). Finally, accumulation of FOXO1 in the nucleus was measured by immunofluorescence and Western blot analysis. As expected, an increase in accumulation and expression of FOXO1 was observed in the nucleus following PARPi treatment compared to gp100-activated cells (FIGS. 7F and 15E). To further validate the implication of SIRT-1/FOXO1 signaling in the generation of T_{CM}^{sup} cells, SIRT-1 and FOXO1 were pharmacologically inhibited in the presence of different PARP inhibitors. SIRT-1 or FOXO1 inhibition significantly reduced PARP inhibitor-mediated generation of T_{CM}^{sup} cells and IFNγ and Gzm B expression in T_{CM} cells (FIGS. 8A-8F). Together, these results show that PARPi leads to the accumulation of NAD and hence activation of SIRT-1/FOXO1 signaling pathway that enhances metabolic fitness and modulates gene expression, leading to T_{CM}^{sup} cell generation.

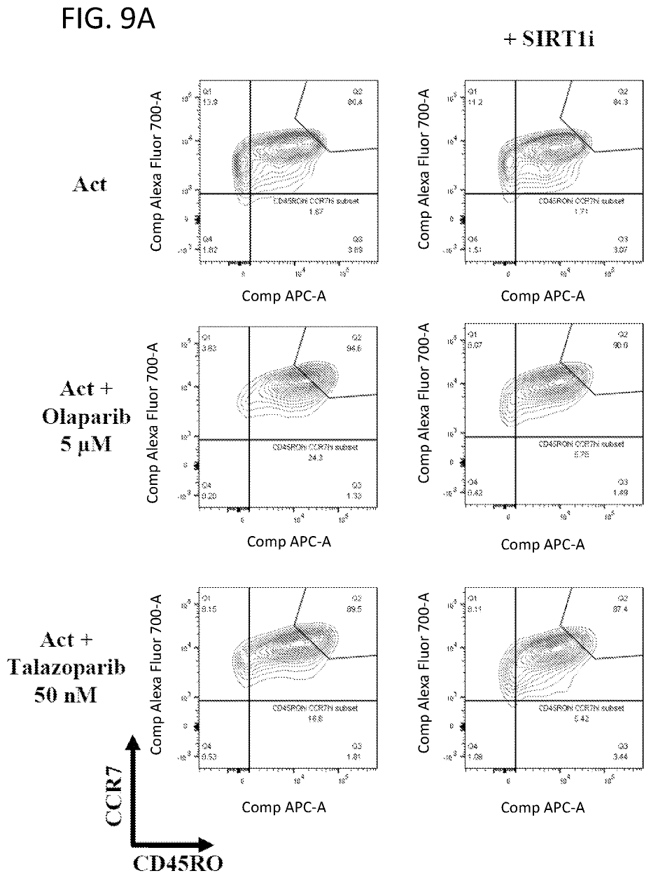

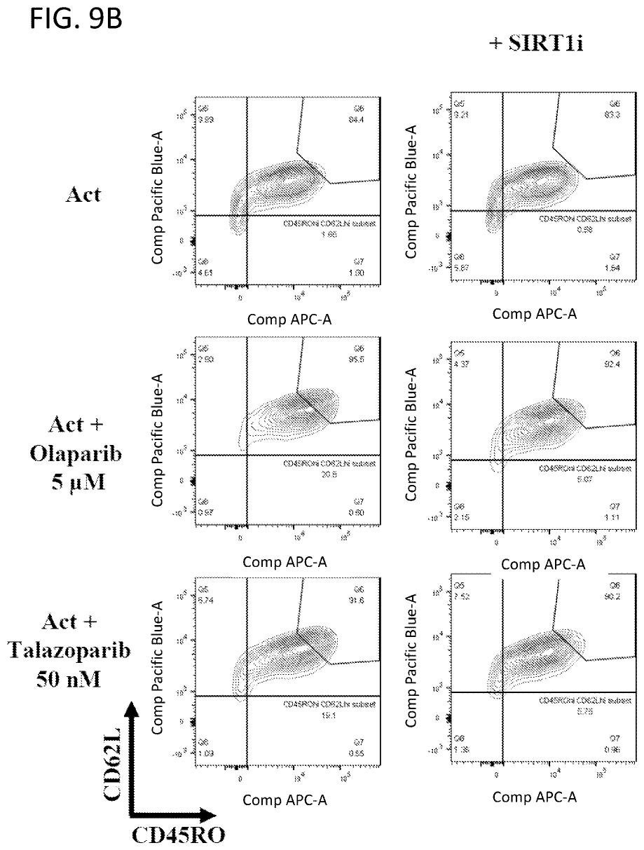

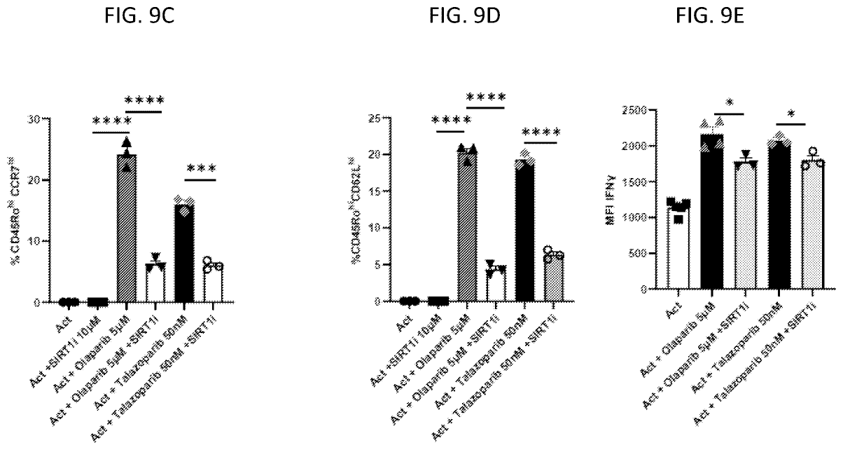

Based on the findings on mouse CD8 T cells, an additional study was performed to check whether PARPi can activate SIRT-1/FOXO1 pathway in human CD8 T cells and thus enhance the generation of T_{CM}^{sup}. To this end, human naïve CD8 T cells were activated with anti-CD3/CD28 coupled beads for 72 h. In line with the findings in mouse CD8 T cells, treatment of activated human CD8 T cells with either olaparib or talazoparib resulted in a significant increase in the number of CD45RO^{hi}CCR7^{hi} and CD45RO^{hi}CD62L^{hi} cells as well as IFNγ expression (FIGS. 9A-9E). This effect was significantly reduced following inhibition of SIRT-1. Together, these results confirm the findings of the ability of PARPi to activate the SIRT-1/FOXO1 pathway in order to generate a super-memory CD8 T cell population, T_{CM}^{sup}.

It will be apparent that the precise details of the methods or compositions described may be varied or modified without departing from the spirit of the described aspects of the disclosure. We claim all such modifications and variations that fall within the scope and spirit of the claims below.

1. An ex vivo method of producing super central memory CD8+ T cells that express CD62L and CD44, CD45RO and CD62L, or CD45RO and CCR7, comprising culturing CD8+ T cells in the presence of a poly(ADP-ribose) polymerase (PARP) inhibitor (PARPi), thereby generating super central memory CD8+ T cells that express CD62L and CD44, CD45RO and CD62L, or CD45RO and CCR7.

2. The method of claim 1, wherein the CD8+ T cells are peripheral blood CD8+ T cells, tumor-infiltrating CD8+ T cells, or engineered CD8+ T cells.

3. The method of claim 1, wherein:
the super central memory CD8+ T cells express CD62L^{int}CD44^{int}, CD45RO^{int} and CD62L^{int}, or CD45RO^{int} and CCR7^{int}; or
the super central memory CD8+ T cells express CD62L^{hi}CD44^{hi}, CD45RO^{hi} and CD62L^{hi}, or CD45RO^{hi} and CCR7^{hi}.

4. (canceled)

5. The method of claim 1, wherein the PARPi is a small molecule inhibitor.

6. The method of claim 5, wherein:
the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide;
the CD8+ T cells are cultured in the presence of about 1 nM to about 15 μM of the small molecule inhibitor; and/or
the CD8+ T cells are cultured in the presence of the small molecule inhibitor for at least 12 hours, at least 24 hours, at least 36 hours, at least 48 hours, about 60 hours, or at least 72 hours.

7-9. (canceled)

10. The method of claim 1, wherein:
the CD8+ T cells are obtained from a subject with cancer; or
the CD8+ T cells are obtained from a healthy donor.

11. (canceled)

12. A method of treating a cancer in a subject, comprising:
culturing CD8+ T cells ex vivo with a PARP inhibitor (PARPi), thereby producing super central memory CD8+ T cells that express CD62L and CD44, CD45RO and CD62L, or CD45RO and CCR7; and
adoptively transferring the super central memory CD8+ T cells to the subject, thereby treating the cancer in the subject.

13. The method of claim 12, wherein:
the CD8+ T cells are autologous CD8+ T cells or the CD8+ T cells are allogeneic CD8+ T cells; and/or
the CD8+ T cells are peripheral blood CD8+ T cells, tumor-infiltrating CD8+ T cells, or engineered CD8+ T cells.

14-15. (canceled)

16. The method of claim 12, wherein:
the super central memory CD8+ T cells express CD62L^{int}CD44^{int}, CD45RO^{int} and CD62L^{int}, or CD45RO^{int} and CCR7^{int}; or
the super central memory CD8+ T cells express CD62L^{hi}CD44^{hi}, CD45RO^{hi} and CD62L^{hi}, or CD45RO^{hi} and CCR7^{hi}.

17. (canceled)

18. The method of claim 12, wherein the PARPi is a small molecule inhibitor.

19. The method of claim 18, wherein:
the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide;
the CD8+ T cells are cultured in the presence of about 1 nM to about 15 μM of the small molecule inhibitor; and/or
the CD8+ T cells are cultured in the presence of the small molecule inhibitor for at least 12 hours, at least 24 hours, at least 36 hours, at least 48 hours, about 60 hours, or at least 72 hours.

20-22. (canceled)

23. The method of claim 12, wherein the subject is further administered an immunotherapy for treating the cancer.

24. The method of claim 23, wherein the immunotherapy comprises:
a monoclonal antibody that specifically binds to a tumor antigen;
immune cells expressing a tumor antigen-specific chimeric antigen receptor (CAR);
a checkpoint inhibitor;
a co-stimulatory agonist antibody;
an oncolytic virus;
a cancer vaccine; or
one or more cytokines.

25-26. (canceled)

27. A method of enhancing an immune response to a vaccine in a subject, comprising:
administering to the subject the vaccine and a therapeutically effective amount of a PARP inhibitor (PARPi); or
administering to the subject the vaccine and a therapeutically effective amount of super central memory CD8+ T cells that express CD62L and CD44, CD45RO and CD62L, or CD45RO and CCR7.

28. The method of claim 27, wherein:
the PARPi is a small molecule inhibitor;
the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide; and/or
the therapeutically effective amount of the PARPi is about 2.5 mg/kg to about 5.0 mg/kg.

29-30. (canceled)

31. The method of claim 27, further comprising producing the super central memory CD8+ T cells that express CD62L and CD44, CD45RO and CD62L, or CD45RO and CCR7 by culturing CD8+ T cells with a PARPi.

32. The method of claim 31, wherein:
the CD8+ T cells are autologous CD8+ T cells or the CD8+ T cells are allogeneic CD8+ T cells; and/or
the CD8+ T cells are peripheral blood CD8+ T cells, tumor-infiltrating CD8+ T cells, or engineered CD8+ T cells.

33-34. (canceled)

35. The method of claim 27, wherein:
the super central memory CD8+ T cells express CD62L^{int}CD44^{int}, CD45RO^{int} and CD62L^{int}, or CD45RO^{int} and CCR7^{int}; or
the super central memory CD8+ T cells express CD62L^{hi}CD44^{hi}, CD45RO^{hi} and CD62L^{hi}, or CD45RO^{hi} and CCR7^{hi}.

36. (canceled)

37. The method of claim 31, wherein the PARPi is a small molecule inhibitor.

38. The method of claim 37, wherein:
the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide;
the CD8+ T cells are cultured in the presence of about 1 nM to about 15 μM of the small molecule inhibitor; and/or
the CD8+ T cells are cultured in the presence of the small molecule inhibitor for at least 12 hours, at least 24 hours, at least 36 hours, at least 48 hours, about 60 hours, or at least 72 hours.

39-41. (canceled)

42. The method of claim 27, wherein the therapeutically effective amount of the super central memory CD8+ T cells that express CD62L and CD44, CD45RO and CD62L, or CD45RO and CCR7 is about 1 to about 10 million T cells/kg.

43. The method of claim 27, wherein the vaccine is a cancer vaccine, a viral vaccine or a bacterial vaccine.

44. (canceled)

45. A method of treating a cancer in a subject, comprising:
administering to the subject an immunotherapy and a therapeutically effective amount of a PARP inhibitor (PARPi), thereby treating the cancer in the subject; or
administering to the subject an immunotherapy and a therapeutically effective amount of super central memory CD8+ T cells that express CD62L and CD44, CD45RO and CD62L, or CD45RO and CCR7.

46. The method of claim 45, wherein the PARPi is a small molecule inhibitor.

47. The method of claim 46, wherein:
the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide; and/or
the therapeutically effective amount of the PARPi is about 2.5 mg/kg to about 5.0 mg/kg.

48. (canceled)

49. The method of claim 45, further comprising producing the super central memory CD8+ T cells that express CD62L and CD44, CD45RO and CD62L, or CD45RO and CCR7 by culturing CD8+ T cells with a PARPi.

50. The method of claim 49, wherein:
the CD8+ T cells are autologous CD8+ T cells or the CD8+ T cells are allogeneic CD8+ T cells; and/or
the CD8+ T cells are peripheral blood CD8+ T cells, tumor-infiltrating CD8+ T cells, or engineered CD8+ T cells.

51-52. (canceled)

53. The method of claim 45, wherein:
the super central memory CD8+ T cells express CD62L^{int}CD44^{int}, CD45RO^{int} and CD62L^{int}, or CD45RO^{int} and CCR7^{int}; or
the super central memory CD8+ T cells express CD62L^{hi}CD44^{hi}, CD45RO^{hi} and CD62L^{hi}, or CD45RO^{hi} and CCR7^{hi}.

54. (canceled)

55. The method of claim 49, wherein the PARPi is a small molecule inhibitor.

56. The method of claim 55, wherein:
the small molecule inhibitor comprises olaparib, niraparib, talazoparib, veliparib, pamiparib, rucaparib, iniparib, saruparib, AG014699, CEP9722, CEP8983, MK4827, INO-1001, E7016, AZD2461, LT-673, PD128763, BGB-290, and/or 3-aminobenzamide;
the CD8+ T cells are cultured in the presence of about 1 nM to about 15 μM of the small molecule inhibitor; and/or
the CD8+ T cells are cultured in the presence of the small molecule inhibitor for at least 12 hours, at least 24 hours, at least 36 hours, at least 48 hours, about 60 hours, or at least 72 hours.

57-59. (canceled)

60. The method of claim 45, wherein the therapeutically effective amount of the super central memory CD8+ T cells that express CD62L and CD44, CD45RO and CD62L, or CD45RO and CCR7 is about 1 to about 10 million T cells/kg.

61. The method of claim 45, wherein the immunotherapy comprises:
a monoclonal antibody that specifically binds to a tumor antigen;
immune cells expressing a tumor antigen-specific chimeric antigen receptor (CAR);
a checkpoint inhibitor;
a co-stimulatory agonist antibody;
an oncolytic virus;
a cancer vaccine; or
one or more cytokines.

62-63. (canceled)


SEQUENCE LISTING (4 sequences)

SEQ ID NO: 1 - protein, 9 aa, synthetic construct
RAHYNIVTF

SEQ ID NO: 2 - protein, 9 aa, synthetic construct
KVPRNQDWL

SEQ ID NO: 3 - protein, 5 aa, synthetic construct
PADRE

SEQ ID NO: 4 - protein, 13 aa, synthetic construct
  SITE 1: note = D-alanine
  MOD_RES 3: note = l-cyclohexylalanine
  SITE 13: note = D-alanine
AKXVAAWTLKAAA